(19) United States
(12) Patent Application Publication
(10) Pub. No.: US 2023/0256057 A1
(43) Pub. Date: Aug. 17, 2023

(54) TREATMENT OF IMMUNE DEPRESSION

(71) Applicant: Transgene, Illkirch Graffenstaden (FR)

(72) Inventors: Geneviève INCHAUSPE, Lyon (FR); Perrine MARTIN, L'isle D'abeau (FR); Stéphane LEUNG-THEUNG-LONG, Toulouse (FR); Karine LELU-SANTOLARIA, Pont-Eveque (FR); Alexei EVLACHEV, Lyon (FR); Charles Antoine COUPET, Pierre Benite (FR); Aurélie RAY, Lyon (FR); Clarisse DUBOIS, Chessy (FR)

(73) Assignee: Transgene, Illkirch Graffenstaden (FR)

(21) Appl. No.: 18/015,820

(22) PCT Filed: Jul. 13, 2021
(86) PCT No.: PCT/EP2021/069463

§ 371 Date: Jan. 12, 2023

(30) Foreign Application Priority Data
Jul. 13, 2020 (EP) 20305798.9

(51) Int. Cl. A61K 38/20; A61K 48/00; A61P 37/04; C12N 15/86

(52) U.S. Cl. CPC A61K 38/2046; A61K 48/0041; A61P 37/04; C12N 15/86; C12N 2710/24143; C12N 2710/24162

(57) ABSTRACT

The present invention is in the field of immunotherapy. The invention provides non-propagative viral vectors comprising a nucleic acid molecule encoding at least a polypeptide having an IL-7 activity, wherein said non-propagative viral vector is for use in the treatment of immune depression induced by sepsis, burn, trauma, major surgery, senescence and/or coronavirus.

### TECHNICAL FIELD OF THE INVENTION

The present invention is in the field of immunotherapy. The invention provides viral vectors comprising a nucleic acid molecule encoding at least a polypeptide for use in the treatment of immune depression. More precisely, the viral vector of the invention is a non-propagative viral vector, the polypeptide has an IL-7 activity, and the immune depression is induced by sepsis, burn, trauma, major surgery, senescence and/or coronavirus. The invention is particularly useful for the treatment of immune depression induced by sepsis.

### BACKGROUND ART

Immunodepression (also referred as immunosuppression or immunoparalysis) is a state of temporary or permanent dysfunction of the immune response. It results from damage to the immune system, leads to increased susceptibility to disease agents and places the patients at a higher risk for infection, sepsis and subsequent mortality. A variety of dysfunctions is associated with the immunodepressed states that affect both specific and nonspecific components of host defense, including abnormal activity of immune effector cells, decreased activation of immunostimulator T-cells, increased suppressor T-cell functions and altered cytokine levels.

A number of documents showed similarities between immunodepression induced by sepsis, trauma, major surgery, burn, senescence and coronavirus, including induction of monocytosis and low HLA-DR on monocytes surface in patients having trauma and sepsis (Heftrig et al., 2017, Mediat Inflamm, article ID 2608349), high production of pro-inflammatory cytokines (e.g. TNFα, IL-6 and IL-8) during senescence and after surgery or trauma (Jia et al., 2019, Eur J Trauma Emerg S, https://doi.org/10.1007/s00068-019-01271-6; Kovac et al., 2005, Exp Gerontol., 40(7):549-55), persistently elevated IL-10 levels in the plasma of patients suffering from sepsis, burn injuries or trauma (Thomson et al., 2019, Military Medical Research, 6:11), and severe lymphopenia affecting all lymphocyte subsets, decreased mHLA-DR and moderately increased plasma cytokine levels showing at the same time both inflammatory (IL-6) and immunosuppressive (IL-10) responses for patients having trauma and coronavirus (Monneret et al., 2020, Intensive Care Med, 46:1764-1765).

Sepsis is defined as a life-threatening organ dysfunction caused by a deregulated host response to an infection, and mortality rates remain high. Nowadays, sepsis represents the leading cause of death in the intensive care units and was declared as a global health priority: resolutions were adopted to improve its prevention, diagnosis and management (Venet and Monneret, 2017, Nat Rev Nephrol., 14(2):121-137).

In sepsis, the release of proinflammatory mediators induces hemodynamic instability, end-organ dysfunction and coagulation abnormalities (Meisel et al., 2009, Am J Respir Crit Care Med., 180(7):640-8). Therefore, until a few years, clinical trials focused on blocking inflammation early in the course of sepsis in order to suppress the host immune system response. However, blocking inflammation failed to improve the sepsis outcome (Hamers et al., 2015, Minerva Anestesiol., 81(4):426-439). It had become clear that a considerable proportion of sepsis patients did not die of an overwhelming immune response and that suppressing the immune system was not an effective strategy when applied to all sepsis patients (Peters van Ton et al., 2018, Front Immunol, 9: 1926).

Investigations were undertaken in order to understand sepsis physiopathology and to reduce sepsis-related mortality. It appeared that the host immune response during and after sepsis is extremely complex: it varies over time, with the concomitant occurrence of both pro-inflammatory and anti-inflammatory mechanisms, and fails to return to homeostasis (Venet and Monneret, 2017, Nat Rev Nephrol., 14(2):121-137). It also appeared that not the initial hyperinflammation state, but rather a profoundly suppressed state of the immune system, called sepsis-induced immunodepression, sepsis-associated immunosuppression or sepsis-associated immunoparalysis, accounts for the majority of sepsis-related deaths (Hamers et al., 2015, Minerva Anestesiol., 81(4):426-439).

Sepsis-induced immunodepression is characterized by impaired innate and adaptive immune responses, including enhanced apoptosis and dysfunction of CD8+ and CD4+ lymphocytes, impaired phagocytic functions, monocytic deactivation with diminished HLA class II surface expression, and altered ex vivo cytokine production (Meisel et al., 2009, Am J Respir Crit Care Med., 180(7):640-8).

Many patients survive the initial hyperinflammatory phase of sepsis but die in the immunosuppressive state. Immunodepression renders the septic patient more vulnerable to secondary opportunistic infections, reactivation of latent infections (Hamers et al., 2015, Minerva Anestesiol., 81(4):426-439) and co-morbidities (e.g. malignancy, cardiovascular diseases, diabetes, renal insufficiency, respiratory insufficiency, etc.).

It appeared that cumulative mortality related to sepsis was 10% after the first week (early mortality), between 20% and 40% 28 days after sepsis (delayed mortality), and between 50% and 70% 3 years after sepsis (long-term mortality) (Venet and Monneret., 2017, Nat Rev Nephrol., 14(2):121-137).

As a result, novel therapeutic strategies aiming at stimulating immune functions were evaluated for the treatment of patients having an immune depression induced by sepsis (Venet and Monneret., 2017, Nat Rev Nephrol., 14(2):121-137), with a growing interest for the development of immunomodulatory-based approaches.

In this regard, a number of immune-stimulatory molecules were tested: GM-CSF, IFNγ, anti-PD-1 and anti-PD-L1 were administered to sepsis-immunodepressed patients, and preliminary studies were conducted with agents like apoptosis inhibitors in animal models. However, despite some effects on the immune system observed in a handful of treated patients or animals (e.g. restoration of monocytic immunocompetence, enhancement of pro-inflammatory cytokine production, increased survival of T and NK cells or potent inhibition of apoptosis), said results were not confirmed on large clinical trials (Peters van Ton et al., 2018, Front Immunol, 9; 1926).

Interleukin-7 is of central importance to the development and homeostasis of the adaptive immune system (Shindo et al., 2015; 43(4): 334-343). As such, IL-7 was proposed for therapy aiming at improving T-cell reconstitution following lymphopenia (Ponchel et al., 2011, Clinica Chimica Acta 412, 7-16). Clinical trials in over 390 oncologic and lymphopenic patients showed that IL-7 was safe, and increased CD4+ and CD8+ lymphocyte counts (Frangois et al., 2018, JCI Insight.; 3(5):e98960).

Preclinical and clinical studies were undertaken to evaluate the capacity of IL-7 to restore immune functions in sepsis-induced immunodepression states. Potential therapeutic effect of recombinant IL-7 was first investigated in two relevant sepsis animal models, respectively a murine intra-abdominal peritonitis model induced by cecal ligation and puncture (CLP) (Unsinger et al., 2010, J Immunol; 184:3768-3779) and a two-hit sepsis model of CLP followed by Candida albicans infection (Shindo et al., 2015 Shock.; 43(4): 334-343). This latter model is thought to mimic the impaired immune status of patients with protracted sepsis who have secondary nosocomial fungal infection (fungal organisms being the third to fourth most common cause of bloodstream infections in many intensive care units). Two rhIL-7 characterized by a long circulating half-life were used in these studies: a rhIL-7 (R&D System) stabilized with an anti-IL-7 antibody and a fully humanized, glycosylated low immunogenic rhIL-7 (called CYT-107, developed by Cytheris, Rockville, Md.). Unsinger reported that both rhIL-7 ameliorated many of the key pathophysiologic processes that are believed to be central to the lethality of sepsis, including a reversion of sepsis-induced depletion of CD4+ and CD8+ T cells, an enhancement of immune effector cell (e.g. lymphocytes) recruitment to the infected site, and a restored IFNγ production, as well as an improved survival for the animals treated with 2 or 3 administrations of rhIL-7 within the 48 hours post CLP (Unsinger et al., 2010, J Immunol; 184:3768-3779).

In contrast, no effect on mouse survival was reported by Shindo et al. in the two-hit sepsis model (CLP followed by Candida albicans) following subcutaneous administrations of CYT-107 for 5 consecutive days beginning 24 hours after C. albicans injection (Shindo et al., 2015 Shock. 43(4): 334-343).

Ex vivo assays conducted on PBMC collected from septic patients and incubated with rhIL-7 confirmed the ability of IL-7 to restore sepsis-induced lymphocyte alterations (Venet et al., 2012, J Immunol November 15, 189 (10) 5073-5081).

More recently, rhIL-7 treatment was clinically investigated in patients with septic shock and severe lymphopenia. Twenty-seven patients received via intramuscular route up to 8 injections of CYT-107 rhIL-7 for 4 weeks. The recombinant IL-7 was well tolerated without evidence of inducing cytokine storm or worsening inflammation or organ dysfunction although specifically grade 1-3 rash was observed at site of injection. rhIL-7 caused an increase in absolute lymphocyte counts and in circulating CD4+ and CD8+ T cells, and increased T cell proliferation and activation. However, rhIL-7 treatment did not improve mortality rate compared to placebo-treated patients (Frangois et al., JCI Insight. 2018; 3(5):e98960).

In contrast to the above-mentioned studies which were conducted with soluble recombinant rhIL-7, thus requiring a strong stabilization to improve its circulating half-life (e.g.: addition of glycosylation sites) and multiple administrations to provide therapeutic effects, the inventors propose a treatment of immune depression based on a vectorized IL-7. A few studies with vectorized immune modulators have been conducted in the field of immune depression induced by sepsis. In particular, Chen et al. administered an adenovirus (Ad) encoding tumor necrosis factor (TNF) in mice after cecal ligation and puncture (CLP), locally or systemically, and concomitantly challenged mice with Pseudomonas aeruginosa. The local administration of Ad TNF significantly improved survival of mice. However, this effect was lost, and even reversed, if Ad TNF was administered systemically: this route of administration increased mice mortality (Chen et al., 2000, J Immunol, 165:6496-6503). The Ad TNF has not been further developed.

As mentioned above, several documents showed similarities between immunodepression induced by sepsis, trauma, major surgery, burn, senescence and coronavirus. Coronaviruses are a group of enveloped viruses with a positive-sense single-stranded RNA genome and a nucleocapsid of helical symmetry. Coronaviruses cause diseases in mammals and birds: in cows and pigs, they cause diarrhea, while in mice they cause hepatitis and encephalomyelitis; in humans and birds, they cause respiratory tract infections that can range from mild to lethal. Mild illnesses in humans include some cases of the common cold, while more lethal varieties can cause SARS, MERS, and COVID-19. COVID-19 is a contagious disease caused by SARS-Cov2. Symptoms of COVID-19 are variable, but often include fever, cough, headache, fatigue, breathing difficulties, and loss of smell and taste. At the forefront of immune alterations previously described in COVID-19, patients homogeneously present severe lymphopenia: lymphocyte count has been associated with increased disease severity in COVID-19, as patients who died from COVID-19 were reported to have had significantly lower lymphocyte counts than survivors.

Monneret et al. conducted an immune monitoring over the first 15 days in COVID-19 patients admitted to intensive care unit. This study showed that immune response to SARS-CoV-2 infection exhibited similarities with the delayed stage of immunosuppression in bacterial sepsis, including severe lymphopenia affecting all lymphocyte subsets, decreased mHLA-DR and moderately increased plasma cytokine levels showing at the same time both inflammatory (IL-6) and immunosuppressive (IL-10) responses. These observations emphasized the potential concern of a persistent immunosuppression during COVID-19 after intensive care unit admission (Monneret et al., 2020, Intensive Care Med, 46:1764-1765). Many treatments have been tested for COVID-19, but none of them have given satisfactory results yet. Here, the inventors propose a treatment of immune depression based on a vectorized IL-7.

### Technical Problem

Insofar as no medical solution exists for the treatment of immune depression induced by sepsis, burn, trauma, major surgery, senescence and/or coronavirus, one may expect that such immune depressions will continue to be a serious global health threat.

As a result, there is a strong medical need in order to restore immune functions in such immune-depressed patients, leading to a reduced associated mortality. More particularly, there is a need for alternatives to IL-7 polypeptides for the treatment of said conditions.

In the context of the present invention, the inventors discovered that non-propagative viral vectors engineered to express IL-7 were an alternative to IL-7 polypeptides, with promising effects on immune system restoration.

Surprisingly, it appeared that a single administration of said non-propagative viral vectors induced IL-7 concentrations within the subjects which were sufficient to restore functional immunity correlating with an increase of splenocytes number and activation, and improved Bcl2 expression of immune cells in spleen and thymus in naïve mice. In CLP models, the administration of said non-propagative viral vectors induced restoration of normal spleen cell counts and restoration of at least partially T cell counts, improvement of blood immune cells counts, activation of several immune cell population in spleen, lungs and blood, boost of T-cell functionality, boost of frequency of cells able to produce TNFα, IL2 or 2 or 3 cytokines among IFNγ, TNFα and IL2, etc. Moreover, in CLP model, the administration of said non-propagative viral vectors significantly increased the host survival, indicating that the combined effects of viral vectors and expressed IL-7 on the immune system could induce an immune-restoration. In COVID-patients, senescent trauma patients, ICU trauma patients and ICU heavy surgery patients, said non-propagative viral vectors induced phosphorylation of STAT5 and/or a boost of T-cell functionality indicating also in these models that the combined effects of viral vectors and expressed IL-7 on the immune system could induce an immune-restauration.

Besides, due to the in situ vector-mediated IL-7 production, one may also expect that said vector will resolve problems encountered with IL-7 polypeptides. Our candidates could improve the patient compliance, with, for example, the reduction of the number of administrations of the treatment. It also could improve the treatment safety, reducing the risk of infections linked to repeated injections.

The technical problem is solved by the provision of the embodiments as defined in the claims.

Other and further aspects, features and advantages of the present invention will be apparent from the following description of the presently preferred embodiments of the invention.

### SUMMARY OF THE INVENTION

One aspect of the invention relates to a non-propagative viral vector comprising a nucleic acid molecule encoding at least a polypeptide having an IL-7 activity, wherein said non-propagative viral vector is for use in the treatment of immune depression induced by sepsis, burn, trauma, major surgery, senescence, and/or coronavirus (i.e. induced by any one of the inducers of immune depression cited herein, or any combination thereof).

In one embodiment, the non-propagative viral vector for use in the invention is a vector selected from the group consisting of poxviruses, adenoviruses, adenovirus associated viruses, vesicular stomatitis viruses, measle virus, poliovirus, Maraba Virus, and viral like particles.

In still another embodiment, the non-propagative viral vector for use in the invention encodes at least a polypeptide selected from the group consisting of the murine IL-7, the human IL-7, the murine IL-7 fused with a Fc (Fc for fragment crystallizable) domain, and the human IL-7 fused with a Fc domain.

In another aspect, the present invention also provides a composition comprising the non-propagative viral vector and an acceptable pharmaceutical vehicle. In one embodiment, the non-propagative viral vector or composition thereof is for use for the treatment of immune depression induced by sepsis, burn, trauma, major surgery, senescence, and/or coronavirus (i.e. induced by any one of the inducers of immune depression cited herein, or any combination thereof). In another embodiment, the composition thereof is administered via intravenous, subcutaneous, mucosal, or intramuscular route.

In still another embodiment, the non-propagative viral vector or composition is for use for increasing the functional innate and/or adaptive immunity in a subject administered with said non-propagative viral vector or composition compared to a subject not administered with said non-propagative viral vector or composition. In another embodiment, the non-propagative viral vector or composition is for use for increasing the functional innate and/or adaptive immunity in a subject administered with said non-propagative viral vector or composition compared to said subject immune response before said non-propagative viral vector or composition administration.

In a preferred embodiment, said use is for increasing the level of at least one type of cells associated with immunity selected from the group consisting of CD4 T cells, CD8 T cells, B cells, NKT cells, NK cells, dendritic cells, monocytes, macrophages, and neutrophils, in a subject administered with said non-propagative viral vector or composition compared to a subject not administered with said non-propagative viral vector or composition. In another embodiment, said use is for increasing the level of at least one type of cells associated with immunity selected from the group consisting of CD4 T cells, CD8 T cells, B cells, NKT cells, NK cells, dendritic cells, monocytes, macrophages, and neutrophils, in a subject administered with said non-propagative viral vector or composition compared to the level of cells associated with immunity of said subject before said non-propagative viral vector or composition administration.

In another preferred embodiment, said use is for increasing the level or the percentage of activated and/or matured cells associated with immunity of at least one type selected from the group consisting of activated CD4 T cells, activated CD8 T cells, activated B cells, activated NK cells, monocytes, and macrophages, in a subject administered with said non-propagative viral vector or composition compared to a subject not administered with said non-propagative viral vector or composition. In another embodiment, said use is for increasing the level or the percentage of activated and/or matured cells associated with immunity of at least one type selected from the group consisting of activated CD4 T cells, activated CD8 T cells, activated B cells, activated NK cells, monocytes, and macrophages, in a subject administered with said non-propagative viral vector or composition compared to the level of activated cells associated with immunity in said subject before said non-propagative viral vector or composition administration.

In a further embodiment, the non-propagative viral vector or composition for use is administered to a subject displaying one or more biomarkers associated with the decrease of the level of cells associated with immunity and/or the level of activated cells associated with immunity.

In a further aspect, the invention also provides a method for treating an immune depression induced by sepsis, burn, trauma, major surgery, senescence, and/or coronavirus (i.e. induced by any one of the inducers of immune depression cited herein, or any combination thereof), in a subject in need thereof comprising one or more administration(s) of a non-propagative viral vector or composition for use as described herein.

### BRIEF DESCRIPTION OF THE FIGURES

FIG. 1. In vitro analysis of the expression of hIL-7-Fc by MVA-hIL-7-Fc (MVATG18897) and functionality assessment.

A. Analysis by Western Blot. A549 cells were infected in vitro by MVA empty (MVATGN33.1, negative control) or MVA-hIL-7-Fc (MVATG18897) and supernatants and cells were collected. hIL-7-Fc expressed in cells and secreted in supernatants were analyzed through Western Blot, in presence or in absence of beta-mercaptoethanol. Produced IL-7 was detected by an anti-IL-7 antibody (rabbit monoclonal antibody specific of human IL-7). B. Analysis of hIL-7-Fc expression through ELISA after in vitro infection. COS7 cells were infected by MVA empty (MVATGN33.1) or MVA-hIL-7-Fc (MVATG18897) at MOI 0.3, 1 or 3. Supernatants of infected cells were collected 48 to 72 hours post infection and expressed hIL-7 was detected following an ELISA. Concentration of detected IL-7 (expressed in ng/mL) is represented on the graph depending on the used MOI and the vector. Black bars correspond to cells infected with MVA-hIL-7-Fc (MVATG18897) and open bars represent cells infected by empty MVA. C. Functionality assessment of the produced IL-7 using PB1 cells. Functionality of produced IL-7 in supernatants was tested by evaluating the effect of the supernatants on PB1 cells, which is a cell-line dependent on IL-7 for its proliferation. After 72 h of incubation the metabolic activity of cells, reflecting their proliferative activity, was assessed using an MTT (3-(4,5-dimethylthiazol-2-yl)-2,5-diphenyltetrazolium bromide) assay. The OD (optical density) measured at the end of MTT assay is presented on the graphs depending on the supernatant tested dilutions. The highest the OD is, the more proliferation of PB1 was induced. Observed OD with supernatants from empty MVA at MOI 0.3, 1 and 3 were represented respectively by open triangles, open squares and open circles and the ones from MVA-hIL-7-Fc (MVATG18897) were represented respectively by black triangles, black squares and black circles for MOI 0.3, 1 and 3.

FIG. 2. Circulating Human IL-7 and Murine IFNγ Pharmacokinetics in Healthy C57BL6/J Mice Injected by 2 Different Doses of MVA-hIL-7-Fc (MVATG18897) (Experiment 1).

Circulating hIL-7 and mIFNγ was measured using a human IL-7 ELISA and a murine IFNγ ELISA using sera samples from injected mice. Blood samples were collected at 0, 2, 6, 24, 48, 72, 96 h, 8, 15 and 21 days. Three mice per timepoint were sampled for all groups. Mean concentrations of detected hIL-7 (ng/mL) are represented by black squares and mean concentrations of detected mIFNγ (ng/mL) are represented by open squares. The dotted line with large points represents the limit of quantification of the mIFNγ ELISA and the dotted line with the smallest points represents the limit of quantification of the hIL-7 ELISA. FIG. 2A shows mean concentration values of circulating hIL-7 and mIFNγ observed following one IV injection of MVA empty (1·10⁸ pfu) overtime. FIG. 2B shows mean concentration values of circulating hIL-7 and mIFNγ observed following one IV injection of MVA-hIL-7-Fc (1.107 pfu) overtime. FIG. 2C shows mean concentration values of circulating hIL-7 and mIFNγ observed following one IV injection of MVA-hIL-7-Fc (MVATG18897) (1·10⁸ pfu) overtime.

FIG. 3. Circulating Human IL-7 and Murine IFNγ Pharmacokinetics in Healthy C57BL6/J Mice Injected by MVA-hIL-7-Fc (MVATG18897) (Experiment 2).

Circulating hIL-7 and mIFNγ were measured using a human IL-7 ELISA and a murine IFNγ ELISA using sera samples from injected mice. Blood samples were collected at 0, 6, 24, 48, 72, 96 h, 7 and 9 days. Three mice per timepoint were sampled for all groups. Mean concentrations of detected hIL-7 (ng/mL) are represented by black squares and mean concentrations of detected mIFNγ (ng/mL) are represented by open squares. The dotted line with large points represents the limit of quantification of the mIFNγ ELISA and the dotted line with the smallest points represents the limit of quantification of the hIL-7 ELISA. FIG. 3A shows mean concentration values of circulating hIL-7 and mIFNγ observed following one IV injection of MVA empty overtime. FIG. 3B shows mean concentration values of circulating hIL-7 and mIFNγ observed following one IV injection of MVA-hIL-7-Fc (MVATG18897) overtime.

FIG. 4. Analysis of Total Number of Spleen Cells in Healthy C57BL6/J Mice Following One IV Injection of MVA-hIL-7-Fc (MVATG18897).

Biological activities of empty MVA (MVATGN33.1) and MVA-hIL-7-Fc (MVATG18897) were assessed at 1, 3, 9 and 29 days post-injection, 3 mice per group were sacrificed and spleens were sampled in order to count total number of splenocytes. Mean values per group and timepoint+/−SD are represented on graphs. Mean values for untreated mice are represented with white bars, mean values of mice treated by MVA empty (MVATGN33.1) are represented by grey bars and mean values of mice treated by MVA-hIL-7-Fc (MVATG18897) are represented by black bars. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 5. Analysis of the Number of Total CD4+ T Cells in Spleen and of the 4 Sub-Populations (Naïve, Acute Effectors, Effector Memory and Central Memory) Following One IV Injection of MVA-hIL-7-Fc (MVATG18897) in Healthy C57BL6/J Mice.

Biological activities of MVA empty (MVATGN33.1) and MVA-hIL-7-Fc (MVATG18897) were assessed at 1, 3, 9 and 29 days post-injection, 3 mice per group were sacrificed and spleens were sampled in order to characterize the total number of CD4+ T cells and each of the following subpopulations: naïve CD4+ T cells (CD3+CD4+CD62L+CD44−), CD4+ T effector memory cells (CD3+CD4+CD62L− CD44+), CD4+ central memory cells (CD3+CD4+CD62L+CD44+) and CD4+ acute effector cells (CD3+CD4+CD62L− CD44−). Cells were prepared, stained and analyzed as described in the materials and method section. FIGS. 5A, 5B, 5C, 5D and 5E represent respectively absolute numbers per spleen of CD4+ T cells, naïve CD4+ T cells, CD4+ T effector memory cells, CD4+ T central memory cells and CD4+T acute effector cells. Mean values per group and timepoint+/−SD are represented on graphs. Mean values for untreated mice are represented with white bars, mean values of mice treated by MVA empty (MVATGN33.1) are represented by grey bars and mean values of mice treated by MVA-hIL-7-Fc (MVATG18897) are represented by black bars. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 6. Analysis of the Number of Total CD8+ T Cells in Spleen and of the 4 Sub-Populations (Naïve, Acute Effectors, Effector Memory and Central Memory) Following One IV Injection of MVA-hIL-7-Fc (MVATG18897) in Healthy C57BL6/J Mice.

Biological activities of MVA empty (MVATGN33.1) and MVA-hIL-7-Fc (MVATG18897) were assessed at 1, 3, 9 and 29 days post-injection, 3 mice per group were sacrificed and spleens were sampled in order to characterize the total number of CD8+ T cells and each of the following subpopulations: naïve CD8+ T cells (CD3+CD8+CD62L+ CD44−), CD8+ T effector memory cells (CD3+CD8+CD62L−CD44+), CD8+ central memory cells (CD3+CD8+CD62L+CD44+) and CD8+ acute effector cells (CD3+CD8+CD62L− CD44−). Cells were prepared, stained and analyzed as described in the materials and method section. FIGS. 6A, 6B, 6C, 6D and 6E represent respectively absolute numbers per spleen of CD8+ T cells, naïve CD8+ T cells, CD8+ T effector memory cells, CD8+ T central memory cells and CD8+T acute effector cells. Mean values per group and timepoint+/−SD are represented on graphs. Mean values for untreated mice are represented with white bars, mean values of mice treated by MVA empty (MVATGN33.1) are represented by grey bars and mean values of mice treated by MVA-hIL-7-Fc (MVATG18897) are represented by black bars. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 7. Analysis of the Expression of Bcl2 Protein (Anti-Apoptotic Protein) in CD4+ and CD8+ T Cells in the Spleen and within Thymic Cells Following One IV Injection of MVA-hIL-7-Fc (MVATG18897) in Healthy C57BL6/J Mice.

Biological activities of MVA empty (MVATGN33.1) and MVA-hIL-7-Fc (MVATG18897) were assessed at 1 and 3 days post injection, 3 mice per group were sacrificed and spleens and thymus were sampled in order to characterize the expression of Bcl2 on T cells from the spleen and on thymic cells. The level of expression was characterized as the mean fluorescence intensity (MFI) of Bcl2 staining observed on CD4+ T cells in spleen (FIG. 7A), CD8+ T cells in spleen (FIG. 7B) and on thymic cells (FIG. 7C). Cells were prepared, stained and analyzed as described in the materials and methods section. Mean values per group and timepoint+/−SD are represented on graphs. Mean values for untreated mice are represented with white bars, mean values of mice treated by MVA empty (MVATGN33.1) are represented by grey bars and mean values of mice treated by MVA-hIL-7-Fc (MVATG18897) are represented by black bars. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 8. Proportion of Cell Subpopulations within the Thymus Following One IV Injection of MVA-hIL-7-Fc (MVATG18897) in Healthy C57BL6/J Mice.

Biological activities of MVA empty (MVATGN33.1) and MVA-hIL-7-Fc (MVATG18897) were assessed at 1 and 3 days post-injection, 3 mice per group were sacrificed and thymus were sampled in order to characterize 4 subpopulations of cells typically present in the thymus: Double Negative (DN) cells (CD4− CD8−), Double Positive (DP) cells (CD4+CD8+), Single Positive (SP) CD4+ cells (CD4+CD8−) and Single Positive (SP) CD8+ cells (CD4− CD8+). Cells were prepared, stained and analyzed as described in the materials and methods section. Proportions of each cell sub-populations are presented here as mean percentages per group and per timepoint (Day 1 on FIG. 8A and Day 3 on FIG. 8B). DP cells are represented by white section, DN cells are represented by black vertically hatched section, SP CD4+ cells are represented by a checkered pattern section and SP CD8+ cells are represented by a black vertically hatched section.

FIG. 9. Analysis of Total Number of Neutrophils and Myeloid Dendritic Cells (mDC) in Spleen Following One IV Injection of MVA-hIL-7-Fc (MVATG18897) in Healthy C57BL6/J Mice.

Biological activities of MVA empty (MVATGN33.1) and MVA-hIL-7-Fc (MVATG18897) were assessed at 1, 3, 9 and 29 days post-injection, 3 mice per group were sacrificed and spleens were sampled in order to characterize the neutrophils (B220−, NK1.1−, CD11b+, CD11c−, Ly6G+) and the myeloid dendritic cells (B220−, NK1.1−, CD11b−, CD11c+). Cells were prepared, stained and analyzed as described in the materials and methods section. FIGS. 9A and 9B represent respectively absolute numbers per spleen of neutrophils and mDC. Mean values per group and timepoint+/−SD are represented on graphs. Mean values for untreated mice are represented with white bars, mean values of mice treated by MVA empty (MVATGN33.1) are represented by grey bars and mean values of mice treated by MVA-hIL-7-Fc (MVATG18897) are represented by black bars. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 10. Analysis of Monocyte Sub-Population (Ly6C^{high}, Ly6C^{int} and Ly6C^{low}) Numbers in Spleen Following One IV Injection of MVA-hIL-7-Fc (MVATG18897) in Healthy C57BL6/J Mice.

Biological activities of MVA empty (MVATGN33.1) and MVA-hIL-7-Fc (MVATG18897) were assessed at 1, 3, 9 and 29 days post-injection, 3 mice per group were sacrificed and spleens were sampled in order to characterize the monocytes divided in 3 sub-populations: Ly6C^{high} monocytes (B220−, NK1.1−, CD11b+, CD11c−, Ly6G, Ly6C^{high}) being pro-inflammatory and mediating phagocytosis, Ly6C^{int} monocytes (B220−, NK1.1−, CD11b+, CD11c−, Ly6G−, Ly6C^{int}) being pro-inflammatory and Ly6C^{low} monocytes (B220−, NK1.1−, CD11b+, CD11c−, Ly6G−, Ly6C^{low}) being patrolling monocytes. Cells were prepared, stained and analyzed as described in the materials and methods section. FIGS. 10A, 10B and 10C represent respectively absolute numbers per spleen of Ly6C^{high}, Ly6C^{int} and Ly6C^{low} monocytes. Mean values per group and timepoint+/−SD are represented on graphs. Mean values for untreated mice are represented with white bars, mean values of mice treated by MVA empty (MVATGN33.1) are represented by grey bars and mean values of mice treated by MVA-hIL-7-Fc (MVATG18897) are represented by black bars. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 11: Survival of CLP Mice Treated with MVA-hIL-7-Fc (MVATG18897)

Mice underwent CLP on day 0 and were injected once intravenously at the retro-orbital sinus with 100 μL of 1×10⁸ pfu of MVA-hIL-7-Fc (MVATG18897) on day 4 post-CLP. Survival curves are shown before (A) and after (B) treatment with MVA-hIL-7-Fc (MVATG18897) in Sham mice (black circle and dotted line), untreated CLP mice (black circle) and CLP mice treated with MVA-hIL-7-Fc (grey square and grey line). Time of MVA-hIL-7-Fc (MVATG18897) treatment is represented by vertical dotted line. Combined results of two independent experiments are shown. Statistical analyses (SAS® 9.4) were performed using log-rank test followed by ad-hoc comparisons between groups using Tukey multiplicity adjustment test.

FIG. 12: Circulating hIL-7 Level after MVA-hIL-7-Fc (MVATG18897) Treatment in CLP Mice

Level of hIL-7 was assessed in serum of Sham mice (white bar, N=5), CLP mice (black bar, N=7) and CLP mice treated with MVA-hIL-7-Fc (MVATG18897) (grey bar, N=15) by hIL-7 ELISA. Results of 2 combined experiments are expressed as the mean±SD value of hIL-7 in pg per mL.

FIG. 13: Level of Circulating IFNγ Following MVA-hIL-7-Fc (MVATG18897) Treatment in CLP Mice

Levels of IFNγ were assessed in the serum of Sham-operated mice (white bar, N=5), CLP mice (black bar, N=7) and CLP mice treated with MVA-hIL-7-Fc (MVATG18897) (grey bar, N=15) by U-PLEX Multiplex assay. Results of 2 combined experiments are shown as the mean±SD value expressed in pg per mL. Statistical analyses were performed using one-way ANOVA test for repeated measures followed by Mann-Whitney test for group-to-group comparison: a p value <0.05 is represented by *, a p value <0.01 is represented by **, and a p value <0.001 is represented by ***.

FIG. 14: Immune Cell Subsets in Spleen of CLP Mice Treated with MVA-hIL-7-Fc (MVATG18897)

Numbers of total splenocytes (A), T cells (CD3⁺) (B), CD4 T cells (CD3⁺ NK1.1⁻ CD4⁺) (C) and CD8 T cells (CD3⁺ NK1.1⁻ CD8⁺) (D) were assessed by flow cytometry using spleen cells of Sham-operated mice (white bar, N=5), CLP mice (black bar, N=7) and CLP mice treated with MVA-hIL-7-Fc (MVATG18897) (grey bar, N=15). Results of 2 combined experiments are shown as the mean±SD value expressed in 10⁶ cells per spleen. Statistical analyses were performed using one-way ANOVA test for repeated measures followed by Mann-Whitney test for group-to-group comparison: a p value <0.05 is represented by *, and a p value <0.01 is represented by **.

FIG. 15: Activation Status of Immune Cells in Spleen of CLP Mice Treated with MVA-hIL-7-Fc (MVATG18897)

Activation status of splenocytes from Sham-operated mice (white bar, N=5), CLP mice (black bar, N=7) and CLP mice treated with MVA-hIL-7-Fc (MVATG18897) (grey bar, N=15) was determined by assessing cell surface CD69 expression. Results of 2 combined experiments are shown as the mean±SD value of CD69⁺ cell number expressed in 10⁶ cells per spleen for B cells (CD19⁺) (A), CD4 T cells (CD3⁺ NK1.1⁻ CD4⁺) (B), CD8 T cells (CD3⁺ NK1.1⁻ CD8⁺) (C) and NK cells (CD3⁻ CD19⁻ NK1.1⁺) (D). Statistical analyses were performed using one-way ANOVA test for repeated measures followed by Mann-Whitney test for group-to-group comparison: a p value <0.05 is represented by *, a p value <0.01 is represented by **, and a p value <0.001 is represented by ***.

FIG. 16: Circulating Immune Cell Subsets in Blood of CLP Mice Treated with MVA-hIL-7-Fc (MVATG18897)

Numbers of total CD3⁺ (CD45⁺ CD3⁺) (A), CD4 T (CD45⁺ CD3⁺ NK1.1⁻ CD4⁺) (B), CD8 T (CD45⁺ CD3+NK1.1⁻ CD8⁺) (C), NKT (CD45⁺ CD3⁺ NK1.1⁺) (D), NK (CD45⁺ CD3⁻ CD19⁻ NK1.1⁺) (E), B (CD45⁺ CD19⁺) (F), CD11c⁺ (CD45⁺ CD19⁻ CD3 NK1.1⁻ CD11c⁺) (G) and CD11b⁺ (CD45⁺ CD19⁻ CD3 NK1.1⁻ CD11b⁺) (H) cells were assessed by flow cytometry in blood of CLP mice treated (grey bar, N=8) or not (black bar, N=3) with MVA-hIL-7-Fc. Results of one experiment are shown as the mean±SD value of cell number per μL of blood. Statistical analyses were performed using Mann-Whitney test: a p value <0.05 is represented by *.

FIG. 17: Activation Status of Immune Blood Cells in CLP Mice Treated with MVA-hIL-7-Fc (MVATG18897)

Activation status of blood cells in CLP mice treated (grey bar) or not (black bar) with MVA-hIL-7-Fc (MVATG18897) was determined by assessing cell surface CD69 expression. Results are shown as the mean±SD value of CD69⁺ cell number per μL of blood (one experiment, CLP: N=3 and CLP+MVA-hIL-7-Fc: N=8) for CD4 T cells (CD45⁺ CD3⁺ NK1.1⁻ CD4⁺) (A), CD8 T cells (CD45⁺ CD3⁺ NK1.1⁻ CD8⁺) (B), B cells (CD45⁺ CD19⁺) (C) and NK cells (CD45⁺ CD3⁻ CD19⁻ NK1.1⁺) (D). Statistical analyses were performed using Mann-Whitney test: a p value <0.05 is represented by *.

FIG. 18: MVA-hIL-7-Fc (MVATG18897) Treatment Increased Frequency of IFNγ-Producing T Cells in CLP Mice

IFNγ-spots forming cells were assessed by IFNγ ELISpot assay using splenocytes of Sham-operated mice (white bar, N=5), CLP mice (black bar, N=4) and CLP mice treated with MVA-hIL-7-Fc (MVATG18897) (grey bar, N=7) cultured with anti-CD3 and anti-CD28 antibodies. Results of one experiment are expressed as mean±SD values of spots-forming unit per 10⁵ cells (A) and mean±SD values of spot size expressed in 10-3 mm² (B). Statistical analyses were performed using one-way ANOVA test for repeated measures followed by Mann-Whitney test for group-to-group comparison: a p value <0.05 is represented by *, and a p value <0.01 is represented by **.

FIG. 19: All Cytokines-Producing CD4 and CD8 T Cells in CLP Mice Treated with MVA-hIL-7-Fc (MVATG18897)

The frequency of CD4 T cells (CD4+) (A) and CD8 T cells (CD8+) (B) in spleen of Sham-operated mice (white bar, N=5), CLP mice (black bar, N=8) and CLP mice treated with MVA-hIL-7-Fc (grey bar, N=15) producing at least one cytokine among IFNγ, TNFα and IL2 after in vitro stimulation with anti-CD3 and anti-CD28 antibodies was measured by a triple intracellular cytokine staining assay. Results of 2 combined experiments are represented as mean±SD values of the frequency of cytokine-positive cells. Statistical analyses were performed using one-way ANOVA test for repeated measures followed by a Mann-Whitney test for group-to-group comparison: a p value <0.05 is represented by *, a p value <0.01 is represented by **, and a p value <0.001 is represented by ***.

FIG. 20: Cytokine-Producing CD4 T Cells in CLP Mice Treated with MVA-hIL-7-Fc (MVATG18897)

The frequency of CD4 T cells in spleen of Sham-operated mice (white bar, N=5), CLP mice (black bar, N=8) and CLP mice treated with MVA-hIL-7-Fc (MVATG18897) (grey bar, N=15) producing IFNγ, TNFα and/or IL2 after in vitro stimulation with anti-CD3 and anti-CD28 antibodies was measured by a triple intracellular cytokine staining assay. Frequencies of CD4 T cells (CD4+) producing either IFNγ/TNFα (A), IL2/TNFα (B) or IFNγ/IL2/TNFα (C) are shown. Results are expressed as mean+/−SD values of the frequency of each cytokine-positive cell subset among all CD4 T cell population. Statistical analyses were performed using one-way ANOVA test for repeated measures followed by a Mann-Whitney test for group-to-group comparison: a p value <0.05 is represented by *, a p value <0.01 is represented by **, and a p value <0.001 is represented by ***.

FIG. 21: Cytokine-Producing CD8 T Cells in CLP Mice Treated with MVA-hIL-7-Fc (MVATG18897)

The frequency of CD8 T cells in spleen of Sham-operated mice (white bar, N=5), CLP mice (black bar, N=8) and CLP mice treated with MVA-hIL-7-Fc (MVATG18897) (grey bar, N=15) producing IFNγ, TNFα and/or IL2 after in vitro stimulation with anti-CD3 and anti-CD28 antibodies was measured by a triple intracellular cytokine staining assay. Frequencies of CD8 T cells (CD8*) producing only IFNγ (A), IFNγ/TNFα (B) and IFNγ/IL2/TNFα (C) are shown. Results are expressed as mean+/−SD values of the frequency of each cytokine-positive cell subset among all CD8 T cell population. Statistical analyses were performed using one-way ANOVA test for repeated measures followed by a Mann-Whitney test for group-to-group comparison: a p value <0.05 is represented by *, a p value <0.01 is represented by **, and a p value <0.001 is represented by ***.

FIG. 22: Pro- and Anti-Inflammatory Cytokines Produced by Activated T Cells Following MVA-hIL-7-Fc (MVATG18897) Treatment

Total splenocytes from Sham-operated mice (white bar, N=5), CLP mice (black bar, N=7) and CLP mice treated with MVA-hIL-7-Fc (MVATG18897) (grey bar, N=15) were cultured in anti-CD3 antibody-precoated plates and IL-1β (A), IL-6 (B), TNFα (C), IFNγ (D) and IL-10 (E) released in supernatants were measured by U-PLEX Multiplex assay. Results of 2 combined experiments are shown as the mean±SD value of each cytokine and expressed in pg per mL. Statistical analyses were performed using one-way ANOVA test for repeated measures followed by a Mann-Whitney test for group-to-group comparison: a p value <0.01 is represented by **, and a p value <0.001 is represented by ***.

FIG. 23: Survival of CLP Mice Treated with MVA-hIL-7-Fc (MVATG18897) or Empty MVA (MVATGN33.1)

Mice underwent CLP on day 0 and were injected once intravenously at the retro-orbital sinus with 100 μL of 1×10⁸ pfu of MVA-hIL-7-Fc (MVATG18897) or empty-MVA (MVATGN33.1) on day 4 post-CLP. Survival curves are shown before (A) and after (B) treatment in CLP mice treated by empty MVA (black circle and black dotted line) and CLP mice treated with MVA-hIL-7-Fc (grey square and grey line). Time of MVA-hIL-7-Fc (MVATG18897) or empty MVA (MVATGN33.1) treatment is represented by vertical dotted line.

FIG. 24: Circulating Immune Cell Subsets in Blood of CLP Mice Treated with MVA-hIL-7-Fc (MVATG18897) or Empty MVA (MVATGN33.1).

Various cell subsets of spleen were assessed by flow cytometry in blood of CLP mice treated empty MVA (black squares, N=6) or MVA-hIL-7-Fc (grey circles, N=9). Numbers of CD8 T (CD45⁺ CD3⁺ NK1.1⁻ CD8⁺) (A), NKT (CD45⁺ CD3⁺ NK1.1⁺) (B), and CD11b⁺ (CD45⁺ CD19⁻ CD3⁻ NK1.1⁻ CD11b⁺) (C) cells were shown here as well as mean±SD value of cell number per μL of blood. Statistical analyses were performed using one-way ANOVA test for repeated measures followed by Mann-Whitney test for group-to-group comparison: a p value <0.05 is represented by *, a p value <0.01 is represented by **.

FIG. 25: Cytokine-Producing CD4 T Cells in CLP Mice Treated with Empty MVA (MVATGN33.1) or MVA-hIL-7-Fc (MVATG18897)

The frequency of CD4 T cells in spleen of CLP mice treated by empty MVA (black squares, N=6) and CLP mice treated with MVA-hIL-7-Fc (MVATG18897) (grey circles, N=9) producing IFNγ, TNFα and/or IL2 after in vitro stimulation with anti-CD3 and anti-CD28 antibodies was measured by a triple intracellular cytokine staining assay. Frequencies of all CD4 T cells (CD4+) producing at least one cytokine (A), all CD4 T cells producing IFNγ, all CD4 T cells producing TNFα and all CD4 T cells producing IL2 (B), and more specifically CD4 T cell producing IFNγ and TNFα, or producing IFNγ and IL2, or producing IL2 and TNFα, or producing IFNγ and IL2 and TNFα (C) are shown here. Individual values and mean+/−SD are represented. Statistical analyses were performed using one-way ANOVA test for repeated measures followed by a Mann-Whitney test for group-to-group comparison: a p value <0.05 is represented by *, a p value <0.01 is represented by **.

FIG. 26: Cytokine-Producing CD8 T Cells in CLP Mice Treated with Empty MVA (MVATGN33.1) or MVA-hIL-7-Fc (MVATG18897)

The frequency of CD8 T cells in spleen of CLP mice treated by empty MVA (black squares, N=6) and CLP mice treated with MVA-hIL-7-Fc (MVATG18897) (grey circles, N=9) producing IFNγ, TNFα and/or IL2 after in vitro stimulation with anti-CD3 and anti-CD28 antibodies was measured by a triple intracellular cytokine staining assay. Frequencies of all CD8 T cells (CD4+) producing at least one cytokines (A), all CD8 T cells producing IFNγ, all CD8 T cells producing TNFα and all CD8 T cells producing IL2 (B), and more specifically CD8 T cell producing only IFNγ and CD8 T cell producing only TNFα (C), or CD8 T cell producing IFNγ and TNFα, or producing IL2 and TNFα, or producing IFNγ and IL2 and TNFα (D) are shown here. Individual values and mean+/−SD are represented. Statistical analyses were performed using one-way ANOVA test for repeated measures followed by a Mann-Whitney test for group-to-group comparison: a p value <0.01 is represented by **.

FIG. 27. Analysis of Total Number of Lung Cells and of Activated (CD69+) NK, CD8+ and CD4+ T Cells in Healthy C57BL6/J Mice Following One IV Injection of Empty MVA (MVATGN33.1) or MVA-hIL-7-Fc (MVATG18897).

Biological activities of empty MVA (MVATGN33.1) and MVA-hIL-7-Fc (MVATG18897) were assessed at day 3 and day 9 post-injection, 10 mice per group were sacrificed and lungs were sampled in order to count total number of lung cells (FIG. 27A), after lung preparation. Activated NK cell (FIG. 27B) and activated CD8+ (FIG. 27C) and CD4+ (FIG. 27D) T cell numbers were also monitored through flow cytometry. Experiment was performed twice, and results were pooled. Individual values and mean values per group and timepoint+/−SD are represented on graphs. Individual values for untreated mice are represented by empty circles, individual values for mice treated with MVA empty (MVATGN33.1) are represented by empty squares and individual values for mice treated with MVA-hIL-7-Fc (MVATG18897) are represented by full black squares. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 28. Analysis of Total Number of Lung Cells and of Activated (CD69+) NK, CD8+ and CD4+ T Cells in Healthy C57BL6/J Mice Following One IV Injection of MVA-hIL-7-Fc (MVATG18897) or hIL-7-Fc Protein.

Biological activities of MVA-hIL-7-Fc (MVATG18897) and hIL-7-Fc protein were assessed on day 3 and day 9 post-injection, 6 mice per group were sacrificed and lungs were sampled in order to count total number of lung cells (FIG. 28A), after lung preparation. Activated NK cell (FIG. 28B) and activated CD8+(FIG. 28C) and CD4+ (FIG. 28D) T cell numbers were also monitored through flow cytometry. Individual values and mean values per group and timepoint+/−SD are represented on graphs. Individual values for untreated mice are represented by empty circles, individual values for mice treated with MVA-hIL-7-Fc (MVATG18897) are represented by full black squares and individual values for mice treated with the IL-7-Fc protein are represented by full black triangles. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 29: Cytokine-Producing CD8 T Cells in Spleens and Lungs of Healthy C57BL6/J Mice Following One IV Injection of MVA-hIL-7-Fc (MVATG18897) or hIL-7-Fc Protein.

The frequency of CD8 T cells producing IFNγ, TNFα and/or IL2 after in vitro stimulation with anti-CD3 and anti-CD28 antibodies, in spleens and lungs of C57BL6/J healthy mice being either untreated or injected once with MVATG18897 or hIL-7-Fc protein by IV route, was assessed by a triple intracellular cytokine staining assay. CD8+ T cells producing only IFNγ in spleen (FIG. 29A) or in lungs (FIG. 29B), CD8+ T cells producing IFNγ and TNFα in spleens (FIG. 29C) or in lungs (FIG. 29D) and CD8+ T cells producing IFNγ, TNFα and IL2 in spleens (FIG. 29E) or un lungs (FIG. 29F) are presented here. Individual values and mean values per group and timepoint+/−SD are represented on graphs. Individual values for untreated mice are represented by empty circles, individual values for mice treated with MVA-hIL-7-Fc (MVATG18897) are represented by full black squares and individual values for mice treated with the IL-7-Fc protein are represented by full black triangles. Statistical analyses using a 2-way ANOVA were performed using GraphPad Prism. P values were calculated using Bonferroni correction for multiple comparison tests and a p value <0.05 is represented by *, a p value <0.01 is represented by **, a p value <0.001 is represented by *** and a p value <0.0001 is represented by ****.

FIG. 30: Survival of CLP Mice Treated with MVA-hIL-7-Fc (MVATG18897) or hIL-7-Fc Protein.

Mice underwent CLP on day 0 and were injected once intravenously at the retro-orbital sinus with 100 μL of 1×10⁷ pfu of MVA-hIL-7-Fc (MVATG18897) or 5 μg of hIL-7-Fc protein on day 4 post-CLP. Survival curves are shown after treatment in untreated CLP mice (full black squares and black line), in CLP mice treated with MVA-hIL-7-Fc (circle half full and dotted line) or treated with hIL-7-Fc (empty circles and dotted line). Time of MVA-hIL-7-Fc (MVATG18897) or hIL-7-Fc protein treatments is represented by vertical dotted line. As control, survival of naïve and untreated mice is also represented by full black diamonds and dotted line.

FIG. 31. Analysis of Activated (CD69+) CD8+ T and B Cells in CLP Mice Following One IV Injection of MVA-hIL-7-Fc (MVATG18897) or hIL-7-Fc Protein.

Biological activities of MVA-hIL-7-Fc (MVATG18897) and hIL-7-Fc protein were assessed at day 3 post-injection (7 days post-CLP), surviving mice in each group at day 7 post-CLP were sacrificed. Spleens were sampled and activated CD8+ T (FIG. 31A) and B (FIG. 31B) cell numbers were monitored through flow cytometry. Individual values and mean values per group and timepoint+/−SD are represented on graphs. Individual values for untreated mice are represented by full black circles, individual values for mice treated with MVA-hIL-7-Fc (MVATG18897) are represented by full black squares and individual values for mice treated with the IL-7-Fc protein are represented by full black triangles. Statistical analyses were performed using one-way ANOVA test for repeated measures followed by a Dunns test for group-to-group comparison: a p value <0.05 is represented by *, a p value <0.01 is represented by **, a p value <0.001 is represented by ***.

FIG. 32 Cytokine-Producing CD8 T Cells in CLP Mice Treated with MVA-hIL-7-Fc (MVATG18897) or with hIL-7-Fc Protein.

The frequency of CD8 T cells in spleens of untreated CLP mice (full black circles), of CLP mice treated with MVA-hIL-7-Fc (MVATG18897) (full black squares) or with hIL-7-Fc protein (full black triangles) producing at least IFNγ (only or with other cytokines) (FIG. 32A), producing only IFNγ (FIG. 32B), producing IFNγ and TNFα (FIG. 32C) or producing IFNγ and TNFα and IL2 (FIG. 32D), after in vitro stimulation with anti-CD3 and anti-CD28 antibodies was assessed by a triple intracellular cytokine staining assay. Individual values and mean+/−SD are represented. Statistical analyses were performed using one-way ANOVA test for repeated measures followed by a Dunns test for group-to-group comparison: a p value <0.05 is represented by *, a p value <0.01 is represented by **, a p value <0.001 is represented by ***.

FIG. 33. In Vitro Analysis of the Expression of hIL-7-Fc by MVA-hIL-7-Fc (MVATG18897 or MVATG19791).

A and B. Analysis by Western Blot. Chicken Embryo Fibroblasts (CEF) (A) or A549 cells (B) were infected in vitro with MVA empty (MVATGN33.1, negative control) or MVA-hIL-7-Fc (MVATG18897 or MVATG19791) and supernatants and cells were collected. hIL-7-Fc expressed in cells and secreted in supernatants were analyzed through Western Blot, in presence or in absence of beta-mercaptoethanol. Produced IL-7 was detected by an anti-IL-7 antibody (rabbit monoclonal antibody specific of human IL-7). C. Analysis of hIL-7-Fc expression through ELISA after in vitro infection. Supernatants of infected cells (CEF or A549 cells) were collected and expressed hIL-7 was detected following an ELISA. Concentration of detected IL-7 (expressed in ng/mL) is represented on the graph. Grey bars correspond to cells infected with MVATG18897, black bars represent cells infected by MVATG19791 and empty bars correspond to cells infected with MVATGN33.1 but are not visible on graphs as no IL-7 was detected in these supernatants.

FIG. 34. Analysis of pSTAT5 Expression in CD3+ T Cells from Whole Blood of COVID19+ Patients Following Ex Vivo Stimulation with Supernatant from MVA-IL-7-Fc-Infected Cells.

pSTAT5 expression in CD3+ T cells was analyzed by flow cytometry following stimulation with supernatants from MVA-hIL-7-Fc (MVATG18897) or empty MVA (MVATGN33.1) infected or uninfected primary hepatocytes. Whole blood was stimulated, stained and analyzed as described in the materials and methods section. Ratio was computed as follows: values obtained with supernatants from MVATG18897 or MVATGN33.1 or uninfected cells were normalized by the value of the unstimulated condition. Mean of ratios of the 6 patients+/−standard deviation are represented on the graph. Mean values of supernatant from MVA-IL-7-Fc (SN IL-7-Fc) infected cells are represented by black bars while the supernatants from empty MVA (SN N33) or uninfected cells (SN NI cell) are represented by grey bars.

FIG. 35. Analysis of Total IFNγ and IFNγ-TNFα-IL-2 Produced by CD4+ T Cells from Whole Blood of COVID19+ Patients Following Stimulation with Supernatants of MVA-hIL-7-Fc Infected Cells.

Functionality of CD4+ T cells was analyzed by intracellular staining following 3 hours ex vivo stimulation with Duractive 1 in addition to supernatants from MVA-hIL-7-Fc (MVATG18897) or empty MVA (MVATGN33.1)-infected or uninfected primary hepatocyte cells. Whole blood was stimulated, stained and analyzed as described in the materials and methods section. Percentage of CD4+ T cells producing IFNγ (A) or IFNγ-TNFα-IL-2 (B) are shown. Ratio was computed as followed: values of Duractive 1+supernatant of MVATG18897 or MVATGN33.1 infected or uninfected cells were normalized by the value of Duractive 1 condition. Mean of ratios of the 6 patients+/−standard deviation are represented on graph. Mean values of MVA-IL-7-Fc supernatant (DA+SN IL-7-Fc) are represented by black bars and those of empty MVA (DA+SN N33) or uninfected cell supernatants (DA +SN NI cell) are represented by grey bars.

FIG. 36. Analysis of CD57 Expression in CD8 T Cells on PBMCs from Senescent Controls and Hip Fractured Patients.

PBMCs from senescent controls and hip fractured patients (HF) were thawed, stained and analyzed by flow cytometry as described in the materials and methods section. Mean values+/−standard deviation of CD57 expression in CD8 T cells are represented.

FIG. 37. Assessment of Senescent Trauma Patient PBMCs Stimulated with Supernatant from MVA-hIL-7-Fc Infected Cells.

A. pSTAT5 analysis. pSTAT5 expression in unstimulated CD3+ cells and following ex vivo stimulation with supernatants from empty MVA (MVATGN33.1, SN N33 in graph) or MVA-hIL-7-Fc (MVATG19791, SN IL-7-Fc in graph) infected cells was analyzed by flow cytometry. PBMCs from senescent controls and hip fractured patients (HF) were thawed, stimulated, stained and analyzed as described in the materials and methods section. B. T cell proliferation. Proliferation proportion in unstimulated CD3+ cells and following ex vivo stimulation with supernatants from empty MVA (SN N33) or MVA-hIL-7-Fc (SN IL-7-Fc) infected cells was analyzed by flow cytometry. PBMCs from senescent controls and hip fractured patients (HF) were thawed and stained, stimulated for 5 days, then stained and analyzed as described in materials and methods. Mean values+/−standard deviation are represented.

FIG. 38. Analysis of Total IFNγ and IFNγ-TNFα-IL-2 Producing CD4+ T Cells from PBMC of Trauma Patients, Following Ex Vivo Stimulation in Presence of Supernatant from MVA-hIL-7-Fc Infected Cells.

Functionality of CD4+ T cells was analyzed by intracellular staining following stimulation with PMA-ionomycin, anti-CD3/anti-CD28 or unstimulated control, in addition to supernatant from MVA-hIL-7-Fc (black) or empty MVA (grey) infected primary hepatocytes or culture medium as control (white). Thawed PBMCs were stimulated, stained, and analyzed as described in the materials and methods section. Percentage of CD4+ T cells producing IFNγ (A) or IFNγ, TNFα and IL-2 (B) are shown. Mean of the 3 patients and +/−standard deviation are represented on graph. SN IL-7-Fc: MVA-IL-7-Fc (MVATG19791) supernatant; SN N33: empty MVA (MVATGN33.1) supernatant; control: culture medium.

FIG. 39. Analysis of Total IFNγ and IFNγ-TNFα-IL-2 Producing CD4+ T Cells from PBMC of Heavy Surgery Patients, Following Ex Vivo Stimulation in Presence of Supernatant from MVA-hIL-7-Fc Infected Cells.

Functionality of CD4+ T cells was analyzed by intracellular staining following stimulation with PMA-ionomycin, anti-CD3/anti-CD28 or unstimulated control, in addition to supernatant from MVA-hIL-7-Fc (black) or empty MVA (grey) infected primary hepatocytes or culture medium as control (white).

Thawed PBMCs were stimulated, stained, and analyzed as described in the materials and methods section. Percentage of CD4+ T cells producing IFNγ (A) or IFNγ, TNFα and IL-2 (B) are shown. Mean of the 3 patients and +/−standard deviation are represented on graph. SN IL-7-Fc: MVA-IL-7-Fc (MVATG19791) supernatant; SN N33: empty MVA (MVATGN33.1) supernatant; control: culture medium.

### DETAILED DESCRIPTION OF THE INVENTION

A number of definitions are provided here that will assist in the understanding of the invention. However, unless otherwise defined, all technical and scientific terms used herein have the same meaning as commonly understood by one of ordinary skill in the art to which this invention belongs. All references cited herein are incorporated by reference in their entirety.

### General Definitions

As used throughout the entire application, the terms “a” and “an” are used in the sense that they mean “at least one”, “at least a first”, or “a plurality” of the referenced components or steps, unless the context clearly dictates otherwise. For example, the term “a non-propagative viral vector” includes a plurality of non-propagative viral vectors, including mixtures thereof.

The term “one or more” refers to either one or a number above one (e.g. 2, 3, 4, 5, etc.).

The term “and/or” wherever used herein includes the meaning of “and”, “or” and “all or any other combination of the elements connected by said term”. For example, “innate and/or adaptive immune system” means either innate immune system or adaptive immune system or both innate and adaptive immune systems.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. “Consisting of” means excluding other components or steps of any essential significance. Thus, a composition consisting of the recited components would not exclude trace contaminants and pharmaceutically acceptable carriers.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (RNA) (e.g. mRNA, antisense RNA, SiRNA) or mixed polyribo-polydeoxyribonucleotides. They encompass single- or double-stranded, linear or circular, natural or synthetic, modified or unmodified polynucleotides.

The term “polypeptide” is to be understood to be a polymer of at least nine amino acid residues bonded via peptide bonds regardless of its size and the presence or not of post-translational components (e.g. glycosylation). No limitation is placed on the maximum number of amino acids comprised in a polypeptide. As a general indication, the term refers to both short polymers (typically designated in the art as peptide) and to longer polymers (typically designated in the art as polypeptide or protein). This term encompasses native polypeptides, modified polypeptides (also designated derivatives, analogues, variants or mutants), polypeptide fragments, polypeptide multimers (e.g. dimers), fusion polypeptides among others. The term also refers to a recombinant polypeptide expressed from a polynucleotide sequence that encodes said polypeptide. Typically, this involves translation of the encoding nucleic acid into a mRNA sequence and translation thereof by the ribosomal machinery of the cell to which the polynucleotide sequence is delivered.

The term “identity” refers to an amino acid to amino acid or nucleotide to nucleotide correspondence between two polypeptides or nucleic acid sequences. The percentage of identity between two sequences is a function of the number of identical positions shared by the sequences, taking into account the number of gaps that need to be introduced for global optimal alignment and the length of each gap. Various computer programs and mathematical algorithms are available in the art to determine the percentage of identity between amino acid sequences, such as for example the Blast program available at NCBI or ALIGN in Atlas of Protein Sequence and Structure (Dayhoffed, 1981, Suppl., 3: 482-9). Programs for determining identity between nucleotide sequences are also available in specialized data base (e.g. Genbank, the Wisconsin Sequence Analysis Package, BESTFIT, FASTA and GAP programs). Those skilled in the art can determine appropriate parameters for measuring alignment including any algorithms needed to achieve maximum alignment over the sequences to be compared. For illustrative purposes, “at least 80% identity” means 80% identity or above (81%, 82%, 83%, 84%, 85%, 86%, 87%, 88%, 89%, 90%, 91%, 92%, 93%, 94%, 95%, 96%, 97%, 98%, 99% or 100% identity) whereas “at least 90% identity” means 90%, 91%, 92%, 93%, 94%, 95%, 96%, 97%, 98%, 99% or 100% identity and “at least 95% identity” means 95%, 96%, 97%, 98%, 99% or 100% identity.

The term “obtained from”, “originating” or “originate” and any equivalent thereof is used to identify the original source of a component (e.g. polypeptide, nucleic acid molecule, virus, vector, etc.) but is not meant to limit the method by which the component is made which can be, for example, by chemical synthesis or recombinant means.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. This term also includes cells that can be or has been the recipient of the non-propagative viral vector for use in the invention, as well as progeny of such cells.

The term “subject” generally refers to an organism for whom any non-propagative viral vector, composition and method described herein is needed or may be beneficial. Typically, the organism is a mammal, particularly a mammal selected from the group consisting of domestic animals, farm animals, sport animals, and primates. Preferably, the subject is a human who has been diagnosed as having or at risk of having an immune depression. The terms “subject” and “patient” may be used interchangeably when referring to a human organism and encompasses male and female. The subject to be treated may be a newborn, an infant, a young adult, an adult or an elderly.

The term “treatment” (and any form of treatment such as “treating”, “treat”) as used herein encompasses prophylaxis (e.g. preventive measure in a subject at risk of having an immune depression to be treated and induced by sepsis, burn, trauma, major surgery, senescence, and/or coronavirus) and/or therapy (e.g. in a subject diagnosed as having an immune depression induced by sepsis, burn, trauma, major surgery, senescence, and/or coronavirus), optionally in association with conventional therapeutic modalities. The result of the treatment is to slow down, cure, ameliorate or control the progression of immune depression. For example, a subject is successfully treated for an immunodepression induced by sepsis, burn, trauma, major surgery, senescence and/or coronavirus if after administration of a non-propagative viral vector as described herein, the subject shows an observable improvement of its innate and/or adaptive immunity or immune response and/or recovers.

The term “innate immunity” or “non-specific immunity” as used herein refers to the nonspecific first line of defense against foreign pathogens. Said innate immunity is mediated by cells comprising dendritic cells (DC), natural killer (NK) cells, natural killer T (NKT) cells, monocytes, macrophages, neutrophils, basophils, eosinophils and mast cells. Other elements, like the defense mechanisms of skin, stomach acid and chemicals in the bloodstream are also part of innate immunity.

The term “adaptive immunity” or “specific immunity” as used herein refers to a pathogen-specific immunity, mediated by B lymphocytes, CD4+ helper T lymphocytes, CD8+ cytotoxic T lymphocytes expressing antigen-specific receptors and natural killer (NK) cells. Said adaptive immunity creates memory and allows a flexible and broad repertoire of responses.

The term “administering” (or any form of administration such as “administered”) as used herein refers to the delivery to a subject of a prophylactic or a therapeutic agent such as the non-propagative viral vector described herein.

The term “combination” or “association” as used herein refers to any arrangement possible of various components (e.g. a non-propagative viral vector as described herein and one or more substance effective in improving innate and/or adaptive immunity or immune response). Such an arrangement includes mixture of said components as well as separate combinations for concomitant or sequential administrations. The present invention encompasses combinations comprising equal molar concentrations of each component as well as combinations with very different concentrations. It is appreciated that optimal concentration of each component of the combination can be determined by the artisan skilled in the art.

One aspect of the invention relates to a non-propagative viral vector comprising a nucleic acid molecule encoding at least a polypeptide having an IL-7 activity, wherein said non-propagative viral vector is for use in the treatment of immune depression induced by sepsis, burn, trauma, major surgery, senescence and/or coronavirus.

Viral Vectors

The terms “viral vector”, “virus”, “virion”, “viral particle” and “viral vector particle” are used interchangeably to refer to viral particles that are formed when the genomic nucleic acid is transduced into an appropriate cell or cell line according to suitable conditions allowing the generation of viral particles.

The term “non-propagative viral vector” refers to viral vectors that are unable to propagate in host cells or tissues. These viral vectors can be replication-defective or replication-impaired vectors (e.g. viral vector genetically disabled), meaning that they cannot replicate to any significant extent in normal cells, especially in normal human cells, thus impeding viral vector propagation. The impairment or defectiveness of replication functions can be evaluated by conventional means, such as by measuring DNA synthesis and/or viral titer in non-permissive cells. The viral vector can be rendered replication-defective by partial or total deletion or inactivation of regions critical to viral replication. Such replication-defective or impaired viral vectors typically require for propagation, permissive cell lines which bring up or complement the missing/impaired functions. These viral vectors can also be replication-competent or replication-selective vectors (e.g. engineered to replicate better or selectively in specific host cells) able to produce a first generation of viral particles in the host infected cells, but wherein said first generation of viral particles are unable to infect new host's cells, thus impeding viral vectors propagation. This impairment can be the result of various processes, like the diminution or impairment of DNA production, the diminution or impairment of viral proteins production, the inhibition of scaffold assembly proteins, the uncomplete viral particle maturation, the inability for said viral particles to get out of host cells or to enter new host cells, etc.

Representative examples of suitable viruses for use in this invention are generated from a variety of different virus families (e.g. adenoviridae, papillomaviridae, polyomaviridae, herpesviridae, poxviridae, hepadnaviridae, picornaviridae, coronaviridae, filoviridae, paramyxoviridae, rhabdoviridae, orthomyxoviridae, arenaviridae, bunyaviridae, retroviridae, reoviridae, parvoviridae, flaviviridae etc.).

In one embodiment, non-propagative viral vectors for use in the invention are selected from the group consisting of poxvirus, adenovirus (Ad), adenovirus associated virus (AAV), Vesicular Stomatitis Virus (VSV), measle virus (MV), poliovirus (PV), Maraba Virus and viral like particles. Although one may use wild type or native viruses (i.e. found in nature), preference is given in the context of the present invention to viral like particles and genetically engineered viruses (i.e. a virus that is modified compared to a wild type strain of said virus, e.g. by truncation, deletion, substitution and/or insertion of one or more nucleotide(s) contiguous or not within the viral genome, notably in one or more gene required for viral replication). Modification(s) can be within endogenous viral genes (e.g. coding and/or regulatory sequences) and/or within intergenic regions, preferably resulting in a modified viral gene product. Modification(s) can be made in a number of ways known to those skilled in the art using conventional molecular biology techniques.

Preferably, the modifications encompassed by the present invention affect, for example, virulence, toxicity or pathogenicity of the viral vector compared to a viral vector without such modification, but do not completely inhibit infection and production of new viral particles at least in permissive cells. Said modification(s) preferably lead(s) to the synthesis of a defective protein (or lack of synthesis) so as to be unable to ensure the activity of the protein produced under normal conditions by the unmodified gene. Other suitable modifications include the insertion of exogenous gene(s) (i.e. exogenous meaning not found in a native viral genome), such as a nucleic acid molecule encoding at least a polypeptide having an IL-7 activity as described hereinafter.

A particularly suitable non-propagative viral vector for use in the invention is obtained from a poxvirus. As used herein the term “poxvirus” refers to a virus belonging to the Poxviridae family with a preference for the Chordopoxvirinae subfamily directed to vertebrate host which includes several genus such as Orthopoxvirus, Capripoxvirus, Avipoxvirus, Parapoxvirus, Leporipoxvirus and Suipoxvirus. Orthopoxviruses are preferred in the context of the present invention as well as the Avipoxviruses including Canarypoxvirus (e.g. ALVAC) and Fowlpoxvirus (e.g. the FP9 vector). Sequences of the genome of various Poxviridae, are available in the art in specialized databanks such as Genbank. For example, the vaccinia virus strains Western Reserve, Copenhagen, Cowpoxvirus and Canarypoxvirus genomes are available in Genbank under accession numbers NC_006998, M35027, NC_003663, NC_005309, respectively.

In a preferred embodiment, the non-propagative viral vectors for use in the invention belong to the Orthopoxvirus genus and even more preferably to the vaccinia virus (VV) species. In the native context, Vaccinia viruses are large, complex, enveloped viruses with a linear, double-stranded DNA genome of approximately 200 kb in length which encodes numerous viral enzymes and factors that enable the virus to replicate independently from the host cell machinery. Two distinct infectious viral particles exist, the intracellular IMV (for intracellular mature virion) surrounded by a single lipid envelop that remains in the cytosol of infected cells until lysis and the double enveloped EEV (for extracellular enveloped virion) that buds out from the infected cell. Any vaccinia virus strain can be used in the context of the present invention including, without limitation, MVA (Modified vaccinia virus Ankara), NYVAC, Copenhagen (Cop), Western Reserve (WR), Wyeth, Lister, LIVP Tashkent, Tian Tan, Brighton, Ankara, LC16M8, LC16M0 strains, etc., and any derivative thereof. The gene nomenclature used herein is that of Copenhagen Vaccinia strain. It is also used herein for the homologous genes of other poxviridae unless otherwise indicated. However, gene nomenclature may be different according to the poxvirus strain but correspondence between Copenhagen and other vaccinia strains are generally available in the literature.

Engineered poxviruses can be used with modifications aimed at improving safety (e.g. increased attenuation) and/or efficacy, and/or tropism of the resulting virus. One may cite also defective modifications within the thymidine kinase (J2R; see Weir and Moss, 1983, Genbank accession number AAA48082), the deoxyuridine triphosphatase (F2L), the viral hemagglutinin (A56R), the small (F4L) and/or the large (I4L) subunit of the ribonucleotide reductase, the serine protease inhibitor (B13R/B14R), the complement 4b binding protein (C3L), the scaffold assembly protein (D13L), and within genes like K1L, C7L, A39R and B7R-B8R.

A particularly appropriate non-propagative viral vector for use in the context of the present invention is MVA, due to its highly attenuated phenotype (Mayr et al., 1975, Infection 3: 6-14; Sutter and Moss, 1992, Proc. Natl. Acad. Sci. USA 89: 10847-51). For illustrative purposes, MVA has been generated through serial passages in chicken embryo fibroblasts. Sequence analysis of its genome showed that it has lost the pathogenicity of its parental virus, the Chorioallantois Vaccinia virus Ankara, through alterations of its genome. (Antoine et al., 1998, Virol. 244: 365-96 and Genbank accession number U94848). MVA has been used safely and effectively for smallpox vaccination in more than a hundred thousand individuals. Replicative potential of the virus in human cells is defective but not in chicken embryo cells. Various cellular systems are available in the art to produce large quantities of the virus, notably in egg-based manufacturing processes (e.g. WO2007/147528). Said MVA is also particularly appropriated because of a more pronounced IFN-type 1 response generated upon infection compared to non-attenuated vectors, and of the availability of the sequence of its genome in the literature (Antoine et al., 1998, Virol. 244: 365-96) and in Genbank (under accession number U94848).

Another particularly appropriate non-propagative viral vector for use in the context of the present invention is NYVAC, also due to its highly attenuated phenotype (Tartaglia et al., 1992, Virol. 188(1):217-32). For illustrative purpose, NYVAC is a highly attenuated vaccinia virus strain, derived from a plaque-cloned isolate of the Copenhagen vaccine strain by the precise deletion of 18 open reading frames (ORFs) from the viral genome.

Still another suitable non-propagative viral vector for use in the context of the present invention is a vaccinia virus engineered to be non-propagative, with a specific preference for a non-propagative vaccinia virus of Copenhagen strain having a D13L deletion.

Still another suitable non-propagative viral vector for use in the context of the present invention is an adenovirus (Ad), preferably originating from a human or an animal adenovirus (e.g. canine, ovine, simian, etc.). Any serotype can be employed. Desirably, the adenoviral vector originates from a human adenovirus, or from a chimpanzee adenovirus. Representative examples of chimp Ad include without limitation ChAd3 (Peruzzi et al., 2009, Vaccine 27: 1293), ChAd63 (Dudareva et al., 2009, Vaccine 27: 3501), AdC6, AdC7 (Cervasi et al., 2015, J. of Virology, 87(17):9420-9430, Chen et al., 2015, J. of Virology, 84(20): 10522-10532), ChAdOx1 (Dicks et al., 2012, PLoS One.; 7(7): e40385) and any of those described in the art (see for example WO03/000283; WO03/046124; WO2005/071093; WO2009/073103; WO2009/073104; WO2009/105084; WO2009/136977 and WO2010/086189). Preferably, said non-propagative viral vector for use is a human adenovirus, preferably selected from the group consisting of species A, B, C, D, E, F and G, with a preference for species B, C and D. Said human adenovirus is preferably selected from the group consisting of serotypes 1, 2, 3, 5, 6, 7, 8, 9, 10, 11, 13, 14, 15, 16, 17, 19, 20, 21, 22, 23, 24, 25, 26, 27, 28, 29, 30, 32, 33, 34, 35, 36, 37, 38, 39, 42, 43, 44, 45, 46, 47, 48, 49, 50, 51, 53, 54, 55, 56 and 57, and is more preferably from the group consisting of serotypes 5, 11, 26 and 35.

Replication-defective adenoviruses can be obtained as described in the art, e.g. by deletion of at least a region of the adenoviral genome or portion thereof essential to the viral replication, with a specific preference for partial or total deletion of the E1 region (E1A and/or E1B) comprising E1 coding sequences. The present invention also encompasses viruses having additional deletion(s)/modification(s) within the adenoviral genome (e.g. all or part of regions, like non-essential regions as E3 region, or essential regions as E2 and E4, as described in Lusky et al., 1998, J. Virol 72: 2022; WO94/28152; WO03/104467; Capasso et al., 2014, Viruses, 6, 832-855; Yamamoto et al., 2017, Cancer Sci 108 (2017) 831-837). In a preferred embodiment, the non-propagative viral vector for use in the invention is a human adenovirus 5 which is defective for E1 function (e.g. with a deletion extending from approximately positions 459 to 3510 or 455 to 3512 by reference to the sequence of the human Ad5 disclosed in the GenBank under the accession number M_73260 and in Chroboczek et al. (1992, Virol. 186:280) and further deleted within the E3 region (e.g. with a deletion extending from approximately positions 28591 to 30469 by reference to the same Ad5 sequence).

Polypeptide Having an Interleukin-7 Activity

In one embodiment, the non-propagative viral vector for use according to the invention comprises a nucleic acid molecule encoding at least a polypeptide having an IL-7 activity. The term “IL-7”, “IL7”, “interleukin-7” or “interleukin 7” are used herein interchangeably.

IL-7 is a cytokine having a central activity in the adaptive immune system (Gao et al., 2015, Int J Mol Sci.; 16(5): 10267-10280). It stimulates the differentiation of multipotent hematopoietic stem cells into lymphoid progenitor cells, plays a significant role at specific stages in the development of lymphocytes, and more specifically of T-lineage, B-lineage, and natural killer cells. IL-7 is required for T cell development and expansion, and for maintaining and restoring homeostasis of mature T cells. IL-7 regulates T cell homeostasis at various developmental stages and through three immune modulation pathways: thymic differentiation, peripheral expansion, and extrathymic differentiation. IL-7 also drives T cell proliferation and prompts the survival of naïve and memory T cells in the periphery. To regenerate peripheral T cells, IL-7 directs T cell differentiation and maturation in the thymus. IL-7 is also important in early B cell differentiation because it promotes the commitment of common lymphoid progenitor to the B-lineage. It also acts in concert with transcription factors to regulate immunoglobulin gene rearrangement in the pro-B cell and early pre-B cell stages. Successfully rearranged cells then proliferate in response to IL-7 and other cytokines (Vanloan et al., 2017, J. Immunol. Res., Article ID 4807853).

Like other members of the γ chain (γc-CD132) family of cytokines, IL-7 signals via a ternary complex formed with its unique α-receptor, IL-7Rα (CD127), and the common γc receptor. This interaction stimulates the Janus kinase (JAK) and signal transducer and activator of transcription (STAT) proteins with subsequent activation of the phosphor-inositol 3-kinase (PI3K)/Akt, or Src pathways to facilitate target gene transcription. Engagement of this early pathway by IL-7 ultimately leads to increase of T cell survival and proliferation. The receptor is expressed on various immune cells, including immature B cells, early thymocyte progenitors, and most mature T lymphocytes. More particularly, this receptor is expressed continuously on most resting human T cells with high levels on naive and central memory cells and lower levels on T-reg. IL-7R signal transduction is important in directing the differentiation, proliferation, and survival of immune cells including B, T, and natural killer cells.

A polypeptide having an IL-7 activity refers to a polypeptide providing at least one of the immune effector functions of a native IL-7, notably at least one of an immune function selected from the group consisting of the differentiation, proliferation, activation, and survival of immune cells including B cells, T cells and natural killer cells. Representative examples of polypeptides having an IL-7 activity include native IL-7 polypeptides (e.g. IL-7 polypeptides naturally occurring), modified IL-7 polypeptides (e.g. modified IL-7, derivatives, analogues, variants or mutants of a naturally-occurring IL-7 comprising one or more amino acid modification(s)), IL-7 polypeptide fragments (e.g. a truncated IL-7), IL-7 polypeptide multimers (e.g. dimers), IL-7 fusion polypeptides, and analogues thereof, provided that such polypeptides retain a substantial IL-7 activity (at least 50% of the wild-type counterpart). A vast number of IL-7 analogues is available in the art and can be used in the context of this invention. A particularly appropriate analogue comprises the fusion of IL-7 with a Fc-domain (IL-7-Fc) to improve the stability of IL-7 as described in Seo et al., 2014 J. of Virology 88(16): 8998-9009, Choi et al., 2016, Clin Cancer Res; 22(23): 5898-5908, and Nam et al., 2010, Eur. J. Immunol., 40: 351-358. The Fc-domain, or fragment crystallizable domain, is the tail region of an immunoglobulin that has the ability to interact with cell surface receptors called Fc receptors and some proteins of the complement system. The Fc-domain can originate from an immunoglobulin of class A (IgA), D (IgD), E (IgE), G (IgG) or M (IgM). Preferably, the Fc isoforms are selected for inducing low antibody-dependent cellular cytotoxicity (ADCC) (e.g. murine IgG1, human IgG2).

In one embodiment, IL-7 encoded by the non-propagative viral vector for use herein has the capacity to promote the innate and/or the adaptive response in a subject.

IL-7 can originate from any organism, preferably from a murine (NP_032397 and NP_000871), simian (e.g. G7PC28_MACFA, G7MZL5_MACMU) or human (IL7RA_HUMAN) organism.

The human IL-7 gene locus is 72 kb in length, resides on chromosome 8q12-13 and encodes a protein of 177 amino acids with a molecular weight of 20 kDa. While the murine IL-7 gene is 41 kb in length, it encodes a 154 amino acids protein with a molecular weight of 18 kDa. The primary protein sequences of wild-type murine IL-7 and human IL-7 are disclosed in Genbank under the accession numbers NP_032397 and NP_000871 respectively. In the native context, IL-7 is heavily glycosylated and has a molecular mass of 25 kDa.

IL-7 encoding nucleic acid molecules may be easily obtained by cloning, by PCR or by chemical synthesis based on the information provided herein and the general knowledge of the skilled person. The IL-7-encoding nucleic acid molecules for use herein may be native IL-7-encoding sequences (e.g. cDNA) or analogues thereof derived from the latter by mutation, deletion, substitution and/or addition of one or more nucleotides. Moreover, the IL-7-encoding nucleic acid molecules can be optimized for providing high level expression of the proteins, and/or improving their persistence and/or extending their half-life in a particular host cell or subject as described hereinafter.

In a preferred embodiment, the non-propagative viral vector for use in the invention encodes a polypeptide selected from the group consisting of the murine IL-7 (mIL-7), the human IL-7 (hIL-7), the murine IL-7 fused with a Fc domain (mIL-7-Fc) and the human IL-7 fused with a Fc domain (hIL-7-Fc). Preferably, said murine IL-7 comprises an amino acid sequence having at least 70%, preferably at least 80%, more preferably at least 90% and even more preferably at least 95% identity with the amino acid sequence shown in SEQ ID NO: 1. Preferably, said human IL-7 comprises an amino acid sequence having at least 70%, preferably at least 80%, more preferably at least 90% and even more preferably at least 95% identity with the amino acid sequence shown in SEQ ID NO: 2. Preferably the murine IL-7-Fc comprises an amino acid sequence having at least 70%, preferably at least 80%, more preferably at least 90% and even more preferably at least 95% identity with the amino acid sequence shown in SEQ ID NO: 3. Preferably, the human IL-7-Fc comprises an amino acid sequence having at least 70%, preferably at least 80%, more preferably at least 90% and even more preferably at least 95% identity with the amino acid sequence shown in SEQ ID NO: 4. Codons may be optimized to improve mIL-7, mIL-7-Fc, hIL-7 or hIL-7-Fc protein expression in murine or in human cells, by techniques well known by the person of the art.

The IL-7-encoding nucleic acid molecule can be inserted at any location in the non-propagative viral vector genome. Insertion in any deletion I to VII of the MVA genome is particularly appropriate, with a preference for insertion within deletion II or deletion III, this latter being preferred. The nucleic acid molecule encoding said murine or human IL-7 or IL-7-Fc is placed under the control of appropriate regulatory elements as described hereinafter to allow its expression in the host cell or subject. Insertion in the E1 region is particularly appropriate for adenovirus although insertion in E2 region, E3 region, E4 region or intergenic zones can also be envisaged. The nucleic acid molecule encoding said murine or a human IL-7 or IL-7-Fc is placed under the control of appropriate regulatory elements as described hereinafter to allow its expression in the host cell or subject. Preferably, the IL-7 or IL-7-Fc-encoding nucleic acid molecule is inserted in replacement of the adenoviral E1 region in sense orientation.

In a more preferred embodiment, the present invention relates to a MVA (e.g. with deletion II or deletion 11 as described before, preferably with deletion III) encoding a murine IL-7 (e.g of SEQ ID NO: 1) or human IL-7 (e.g. of SEQ ID NO: 2) or murine IL-7-Fc (e.g. of SEQ ID NO: 3) or human IL-7-Fc (e.g. of SEQ ID NO: 4) for use in the treatment of immune depression induced by sepsis, burn, trauma, major surgery, senescence and/or coronavirus.

Other Molecules of Interest

In one embodiment, the non-propagative viral vector for use in the invention further comprises one or more nucleic acid molecules encoding at least one polypeptide of interest. Examples of polypeptides of interest include cytokines, colony stimulating factors (e.g. GM-CSF, C-CSF, M-CSF, etc.), immunostimulatory polypeptides (e.g. B7.1, B7.2, etc.), immune checkpoint inhibitors (e.g. anti-PD1, anti-PD-L1, anti-CTLA4, etc.), a polypeptide capable of inhibiting a bacterial, parasitic or viral infection or its development (e.g. antigenic polypeptides, antigenic epitopes, trans dominant variants inhibiting the action of a native protein by competition, etc.) or any other polypeptides of interest that are recognized in the art as being useful for the treatment of immune depression induced by sepsis, burn, trauma, major surgery, senescence, and/or coronavirus.

The nucleic acid molecules encoding at least a polypeptide having an IL-7 activity and the additional polypeptide(s) of interest can be placed in the same or in different locations of the non-propagative viral vector genome, like for example in deletion II or in deletion III. Said nucleic acid molecules can be comprised in the same or in different expression cassettes. In the case where they are comprised in the same expression cassette, the nucleic acid molecules can be placed in any order; they also can be fused by a linker, or by self-cleaving peptides. The nucleic acid molecules can be positioned in sense or antisense orientation relative to the natural transcriptional direction of the region in question.

Expression of the IL-7 Encoding Nucleic Acid Molecule

As mentioned before, the IL-7 encoding nucleic acid molecule(s) to be inserted in the genome of the non-propagative viral vector for use as described herein can be optimized for providing high level expression in a particular host cell or subject. It has been indeed observed that the codon usage patterns of organisms are highly non-random, and the use of codons may be markedly different between different genes, cells and hosts. As such nucleic acid(s) may have an inappropriate codon usage pattern for efficient expression in higher eukaryotic cells (e.g. human). Typically, codon optimization is performed by replacing one or more “native” codon corresponding to a codon infrequently used in the host organism of interest by one or more codon encoding the same amino acid which is more frequently used. It is not necessary to replace all native codons corresponding to infrequently used codons since increased expression can be achieved even with partial replacement.

Further to optimization of the codon usage, expression in the host cell or subject can further be improved through additional modifications of the nucleic acid sequence. For example, it may be advantageous to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; and/or internal cryptic regulatory elements such as internal TATA-boxes, chi-sites, ribosome entry sites, and/or splicing donor/acceptor sites.

In accordance with the present invention, the non-propagative viral vector for use comprises the regulatory elements necessary for the expression of a polypeptide having an IL-7 activity in a host cell or subject. The term “regulatory elements” or “regulatory sequences” refers to any element that allows, contributes or modulates expression in a given host cell or subject. The regulatory elements are arranged so that they function in concert for their intended purposes, for example, for a promoter to effect transcription of a nucleic acid molecule from the transcription initiation to the terminator of said nucleic acid molecule in a permissive host cell. In a preferred embodiment, the non-propagative viral vector for use of the invention comprises one or more expression cassettes, each expression cassette comprising at least one promoter placed 5′ to the nucleic acid molecule (e.g. encoding a polypeptide having an IL-7 activity) and one polyadenylation sequence located 3′ to said nucleic acid molecule.

It will be appreciated by those skilled in the art that the choice of the regulatory sequences can depend on such factors as the nucleic acid molecule itself, the non-propagative viral vector into which it is inserted, the host cell or subject to be treated, the level of expression desired, etc. The promoter is of special importance. In the context of the invention, it can be constitutive directing expression of the encoded product (e.g. polypeptide having an IL-7 activity, mIL-7, hIL-7, mIL-7-Fc, hIL-7-Fc) in many types of host cells or specific to certain host cells (e.g. liver-specific regulatory sequences) or regulated in response to specific events or exogenous factors (e.g. by temperature, nutrient additive, hormone, etc.) or according to the phase of a viral cycle (e.g. late or early). One may also use promoters that are repressed during the production step in response to specific events or exogenous factors, in order to optimize viral vector production and circumvent potential toxicity of the expressed polypeptide(s) in the producing cells.

Various promoters may be used in the context of the present invention that are known in the state of the art. Vaccinia virus promoters are particularly appropriate for use in non-propagative poxviral vector (e.g. MVA). Representative examples include, without limitation, the vaccinia p7.5K, pH5R, p11K7.5 (Erbs et al., 2008, Cancer Gene Ther. 15(1): 18-28), TK, p28, p11, B2R, pF17R, pA14L, pSE/L, A35R and K1L promoters, synthetic promoters such as those described in Chakrabarti et al. (1997, Biotechniques 23: 1094-7; Hammond et al, 1997, J. Virol Methods 66: 135-8; and Kumar and Boyle, 1990, Virology 179: 151-8) as well as early/late chimeric promoters. Other promoters may be used, selected from the list comprising cytomegalovirus (CMV) immediate early promoter (U.S. Pat. No. 5,168,062), bidirectional CMV-promoter, Rous sarcoma Virus (RSV) promoter, adenovirus major late (MLP) promoter, phosphoglycero kinase (PGK) promoter (Adra et al., 1987, Gene 60: 65-74), EF1α, thymidine kinase (TK) promoter of herpes simplex virus (HSV)-1, T7 polymerase promoter (WO98/10088) and inducible promoters (e.g. promoters whose transcriptional activity is regulated by the presence or absence of alcohol, tetracycline, steroids, metal, sugar, etc.). CMV promoter is particularly appropriate for use in non-propagative adenoviral vector (e.g. Ad5, Ad11, Ad26, Ad35).

The non-propagative viral vector for use may contain one or more promoters depending on the number of nucleic acid molecule(s) to be expressed. Preferably, when the viral vector encodes a polypeptide having an IL-7 activity and another molecule of interest, each of the encoding nucleic acid molecule is placed under the control of independent promoters. Alternatively, one may use bidirectional promoter. In a preferred embodiment, the nucleic acid molecule encoding the polypeptide having an IL-7 activity is placed under the control of the pH5R promoter.

Those skilled in the art will appreciate that the regulatory elements controlling the nucleic acid expression may further comprise additional elements for proper initiation, regulation and/or termination of transcription (e.g. a transcription termination sequences), mRNA transport (e.g. nuclear localization signal sequences, polyadenylations sequences), processing (e.g. splicing signals, self-cleaving peptides like T2A, P2A, E2A, F2A, linkers), stability (e.g. introns, like 16S/19S or chimeric human p globin/IgG, and non-coding 5′ and 3′ sequences), translation (e.g. an initiator Met, tripartite leader sequences, IRES ribosome binding sites, signal peptides, etc.), targeting sequences, linkers (e.g. linkers composed of flexible residues like glycine and serine), transport sequences, secretion signal, and sequences involved in replication or integration. Said sequences have been reported in the literature and can be readily obtained by those skilled in the art.

Production of the Non-Propagative Viral Vector and Producer Cells

Once generated, the non-propagative viral vector for use herein may be produced/amplified using conventional techniques.

Typically, such viral vectors are produced by a process comprising the steps of (a) introducing the viral vectors into a suitable producer cell line, (b) culturing said cell line under suitable conditions so as to allow the production/amplification of said viral vectors, (c) recovering the produced viral vectors from the culture of said cell line, and (d) optionally purifying said recovered viral vectors.

The choice of the producer cells depends on the type of non-propagative viral vector to be amplified. For example, MVA is strictly host-restricted and is typically amplified on avian cells, either primary avian cells (such as chicken embryo fibroblasts (CEF) prepared from chicken embryos obtained from fertilized eggs) or immortalized avian cell lines. Representative examples of suitable avian cell lines for MVA production include without limitation the Cairina moschata cell lines immortalized with a duck TERT gene (see e.g. WO2007/077256, WO2009/004016, WO2010/130756 and WO2012/001075); avian cell lines immortalized with a combination of viral and/or cellular genes (see e.g. WO2005/042728), spontaneously immortalized cells (e.g. the chicken DF1 cell line disclosed in U.S. Pat. No. 5,879,924), or immortalized cells which derive from embryonic cells by progressive severance from growth factors and feeder layer (e.g. Ebx chicken cell lines disclosed in WO2005/007840 and WO2008/129058 such as Eb66 described in Olivier et al., 2010, mAbs 2(4): 405-15).

For other vaccinia viruses or other poxvirus strains, in addition to avian primary cells (such as CEF) and avian cell lines, many other non-avian cell lines are available for production, including human cell lines such as HeLa (ATCC-CRM-CCL-2™ or ATCC-CCL-2.2™), MRC-5, HEK-293; hamster cell lines such as BHK-21 (ATCC CCL-10), and Vero cells. In a preferred embodiment, non-MVA vaccinia viruses are amplified in HeLa cells (see e.g. WO2010/130753).

For adenoviruses, and more particularly for E1-deleted adenoviral vectors, suitable cell lines include the 293 cells (Graham et al., 1997, J. Gen. Virol. 36: 59-72) as well as the PER-C6 cells and HER96 (e.g. Fallaux et al., 1998, Human Gene Ther. 9: 1909-1917; WO97/00326) or any derivative of these cell lines. But any other cell line described in the art can also be used in the context of the present invention, especially any cell line used for producing product for human use such as Vero cells, HeLa cells and avian cells. Such cells may be adapted for expressing the E1 genes lacking to the defective virus.

Producer cells can be cultured in conventional fermentation bioreactors, flasks, and petri plates. Culturing can be carried out at a temperature, pH and oxygen content appropriate for a given host cell. No attempts will be made here to describe in detail the various prokaryote and eukaryotic host cells and methods known for the production of the non-propagative viral vectors for use in the invention. Producer cells are preferably cultured in a medium free of animal- or human-derived products, using a chemically defined medium with no product of animal or human origin. In particular, while growth factors may be present, they are preferably recombinantly produced and not purified from animal material. An appropriate animal-free medium may be easily selected by those skilled in the art depending on selected producer cells. Such media are commercially available. In particular, when CEFs are used as producer cells, they may be cultivated in VP-SFM cell culture medium (Invitrogen). Producer cells are preferably cultivated at a temperature comprised between 30° C. and 38° C. (more preferably at around 37° C.) for between 1 and 8 days (preferably for 1 to 5 days for CEF and 2 to 7 days for immortalized cells) before infection. If needed, several passages of 1 to 8 days may be made in order to increase the total number of cells.

Infection of producer cell lines by the non-propagative viral vector for use of the invention is made under appropriate conditions (in particular using an appropriate multiplicity of infection (MOI)) to permit productive infection of producer cells.

The infected producer cells are then cultured under appropriate conditions well known to those skilled in the art until progeny viral vectors are produced. Culture of infected producer cells is also preferably performed in a medium (which may be the same as or different from the medium used for culture of producer cells and/or for infection step) free of animal- or human-derived products (using a chemically defined medium with no product of animal or human origin) at a temperature between 30° C. and 37° C., for 1 to 5 days.

The non-propagative viral vectors for use in the invention can be collected from the culture supernatant and/or the producer cell lines. The cell culture supernatant and the producer cells can be pooled or collected separately. Recovery from producer cells (and optionally also from culture supernatant) may require a step allowing the disruption of the producer cell membrane to allow the liberation of the viral vectors. Various techniques are available to those skilled in the art, including but not limited to freeze/thaw, hypotonic lysis, sonication, micro fluidization, or high-speed homogenization. According to a preferred embodiment, the step of recovery of the produced viral vectors comprises a lysis step wherein the producer cell membrane is disrupted, preferably by using a high-speed homogenizer. High speed homogenizers are commercially available from Silverson Machines Inc (East Longmeadow, USA) or Ika-Labotechnik (Staufen, Germany). According to particularly preferred embodiment, said High Speed homogeneizer is a SILVERSON L4R.

The non-propagative viral vectors for use in the invention may then be further purified, using purification steps well known in the art. Various purification steps can be envisaged, including clarification, enzymatic treatment (e.g. endonuclease, protease, etc.), chromatographic and filtration steps. Appropriate methods are described in the art (e.g. WO2007/147528; WO2008/138533, WO2009/100521, WO2010/130753, WO2013/022764). In a preferred embodiment, the purification step comprises a tangential flow filtration (TFF) step that can be used to separate the virus from other biomolecules, to concentrate and/or desalt the virus suspension. Various TFF systems and devices are available in the art depending on the volume to be filtered including, without limitation, Spectrumlabs, Pall Corp, PendoTech and New Pellicon among others.

The non-propagative viral vectors for use in the invention may then be protected by any method known in the art, in order to extend the viral vector persistence in the subject blood circulation. Said methods comprise, but are not limited to, chemical shielding like PEGylation (Tesfay et al., 2013, J. of Virology, 87(7): 3752-3759; N'Guyen et al., 2016, Molecular Therapy Oncolytics, 3, 15021), viroembolization (WO2017/037523), etc.

Host Cells

In another aspect, the invention also relates to host cells which comprise the non-propagative viral vectors for use in the invention. Such host cells encompass producer cells described above.

Viral Composition

The invention also relates to a composition for use in the treatment of immune depression induced by sepsis, burn, trauma, major surgery, senescence, and/or coronavirus (i.e. induced by any one of the inducers of immune depression cited herein, or any combination thereof) in a subject suspected to have or identified as having such an immune depression, wherein said composition comprises at least a therapeutically effective amount of the non-propagative viral vector as described herein or prepared according to the process described herein. Preferably, the composition further comprises a pharmaceutically acceptable vehicle.

A “therapeutically effective amount” corresponds to the amount of the non-propagative viral vector that is sufficient for producing one or more beneficial results. Such a therapeutically effective amount may vary as a function of various parameters, e.g. the mode of administration, the disease state, the age and weight of the subject, the ability of the subject to respond to the treatment, the kind of concurrent treatment and/or the frequency of treatment. The appropriate dosage of viral vector may be routinely determined by a practitioner in the light of the relevant circumstances. Suitably, individual doses for the viral vector may vary within a range extending from approximately 10³ to approximately 10¹² vp (viral particles), iu (infectious unit) or pfu (plaque-forming units) depending on the type of viral vector and quantitative technique used. The quantity of viral vector present in a sample can be determined by routine titration techniques, e.g. by counting the number of plaques following infection of permissive cells (e.g. BHK-21, CEF or HEK-293) (pfu titer), immunostaining quantitative immunofluorescence (e.g. using anti-virus antibodies) (iu titer), by HPLC (vp titer). For illustrative purposes, a suitable dose for a non-propagative poxviral vector is comprised between approximately 10⁶ pfu and approximately 10¹² pfu, more preferably between approximately 10⁷ pfu and approximately 10¹¹ pfu; even more preferably between approximately 10⁸ pfu and approximately 10¹⁰ pfu (e.g. from 5×10⁸ to 6×10⁹, from 6×10⁸ to 5×10⁹, from 7×10⁸ to 4×10⁹, from 8×10⁸ to 3×10⁹, from 9×10⁸ to 2×10⁹ pfu) are convenient for human use, with a preference for individual doses comprising approximately 10⁹ pfu of poxviral vector. Still for illustrative purposes, a suitable dose for a non-propagative adenoviral vector is comprised between approximately 10⁶ and approximately 10¹⁴ vp, preferably between approximately 10⁷ and approximately 10¹³ vp, more preferably between approximately 10⁸ and approximately 10¹² vp, and even more preferably between approximately 10⁹ and approximately 10¹¹ vp (e.g. dose of 10⁹, 2×10⁹, 3×10⁹, 4×10⁹, 5×10⁹, 6×10⁹, 7×10⁹, 8×10⁹, 9×10⁹, 10¹⁰, 2×10¹⁰, 3×10¹⁰, 4×10¹⁰, 5×10¹⁰, 6×10¹⁰, 7×10¹⁰, 8×10¹⁰, 9×10¹⁰, 10¹¹ vp).

The term “pharmaceutically acceptable vehicle” is intended to include any and all carriers, solvents, diluents, excipients, adjuvants, dispersion media, coatings, antibacterial and antifungal agents, absorption agents, and the like compatible with administration in mammals and in particular human subjects.

The non-propagative viral vector for use herein can independently be placed in a solvent or diluent appropriate for human or animal use. The solvent or diluent is preferably isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see for example the most current edition of Remington: The Science and Practice of Pharmacy, A. Gennaro, Lippincott, Williams & Wilkins).

In other embodiments, the viral composition is suitably buffered for human use. Suitable buffers include without limitation phosphate buffer (e.g. PBS), bicarbonate buffer and/or Tris buffer capable of maintaining a physiological or slightly basic pH (e.g. from approximately pH 7 to approximately pH 9).

The composition may also contain other pharmaceutically acceptable excipients for providing desirable pharmaceutical or pharmacodynamic properties, including for example osmolarity, viscosity, clarity, colour, sterility, stability, rate of dissolution of the formulation, modifying or maintaining release or absorption into a human or animal subject, promoting transport across the blood barrier or penetration in a particular organ.

In a further embodiment, the composition may be combined with soluble adjuvants including, but not limited to alum, mineral oil emulsion and related compounds such as those described in WO2007/147529, polysaccharides such as Adjuvax and squalenes, oil in water emulsions such as MF59, double-stranded RNA analogues such as poly(I:C), single stranded cytosine phosphate guanosine oligodeoxynucleotides (CpG) (Chu et al., 1997, J. Exp. Med., 186: 1623; Tritel et al., 2003, J. Immunol., 171: 2358) and cationic peptides such as IC-31 (Kritsch et al., 2005, J. Chromatogr. Anal. Technol. Biomed. Life Sci., 822: 263-70).

In one embodiment, the composition may be formulated with the goal of improving its stability, in particular under the conditions of manufacture and long-term storage (i.e. for at least 6 months, with a preference for at least two years) at freezing (e.g. −70° C., −20° C.), refrigerated (e.g. 4° C.) or ambient temperatures. Various virus formulations are available in the art either in frozen, liquid form or lyophilized form (e.g. WO98/02522, WO01/66137, WO03/053463, WO2007/056847 and WO2008/114021, etc.). Lyophilized compositions are usually obtained by a process involving vacuum drying and freeze-drying. For illustrative purposes, buffered formulations including NaCl and/or sugar are particularly adapted to the preservation of viruses (e.g. S01 buffer: 342.3 g/L saccharose, 10 mM Tris, 1 mM MgCl₂, 150 mM NaCl, 54 mg/L, Tween 80; ARME buffer: 20 mM TRIS, 25 mM NaCl, 2.5% Glycerol (w/v), pH 8.0).

Administration

The non-propagative viral-vector or the composition for use according to the invention, may be administered in a single dose or multiple doses. If multiples doses are contemplated, administrations may be performed by the same or different routes and may take place at the same site or at alternative sites and may comprise the same or different doses in the indicated intervals. Intervals between each administration can be from several hours to 8 weeks (e.g. 24 h, 48 h, 72 h, weekly, every 2 or 3 weeks, monthly, etc.). Intervals can also be irregular. It is also possible to proceed via sequential cycles of administrations that are repeated after a rest period (e.g. cycles of 3 to 6 weekly or bi-weekly administrations followed by a rest period of 3 to 6 weeks). The dose can vary for each administration within the range described above.

Any of the conventional administration routes are applicable in the context of the invention including parenteral, topical or mucosal routes. Parenteral routes are intended for administration as an injection or infusion and encompass systemic as well as locoregional routes. Locoregional administrations are restricted to a localized region of the body (e.g. intraperitoneal or intrapleural administration). Common parenteral injection types are intravenous (into a vein), intra-arterial (into an artery), intradermal (into the dermis), subcutaneous (under the skin) and intramuscular (into a muscle). Infusions typically are given by intravenous route. Topical administration can be performed using transdermal means (e.g. patch and the like). Mucosal administrations include without limitation oral/alimentary, intranasal, intratracheal, intrapulmonary, intravaginal or intra-rectal route. In a preferred embodiment, said composition for use is administered via parenteral route, more preferably via intravenous, subcutaneous or intramuscular route, and even more preferably via intravenous route. In another embodiment, said composition for use is administered via mucosal administration, preferably via intranasal or intrapulmonary routes.

Administrations may use conventional syringes and needles (e.g. Quadrafuse injection needles) or any compound or device available in the art capable of facilitating or improving delivery of the viral vector or composition in the subject.

For illustrative purposes, a suitable composition comprises individual viral dose of 10⁸ pfu to 10¹⁰ pfu (e.g. approximately 10⁹ pfu) of MVA encoding a human IL-7-Fc, preferably for use by intravenous route to the subject in need thereof (e.g. a subject having a sepsis-induced immune depression).

Use and Methods

The present invention provides a non-propagative viral vector or a composition as described herein for use in the treatment of immune depression induced by sepsis, burn, trauma, major surgery, senescence, and/or coronavirus (i.e. induced by any one of the inducers of immune depression cited herein, or any combination thereof), in a subject in need thereof. It also provides a method of treatment comprising administering said viral vector or composition as described herein in an amount sufficient for the treatment of said immune depression.

The terms “immune depression”, “immunodepression”, “immune suppression”, “immunosuppression”, “immune paralysis” or “immunoparalysis” can be used interchangeably. They refer to a state of temporary or permanent dysfunction of the immune response. They also refer to the inability of the body's immune system to work efficiently, to fight infections and other diseases. The inability of the immune system may be partial or complete.

Sepsis is a life-threatening organ dysfunction due to a dysregulated host response to infection. Immune depression induced by sepsis may follow a period of hyper-immune response including an intense increase in cytokine production. The state of said immune depression is life threatening since the immune defense is largely inoperative.

Burn is an injury to tissues caused by contact with dry heat (e.g. fire), moist heat (e.g. steam or liquid), chemicals, electricity, lightning, or radiation.

Trauma is an injury to living tissue caused by an extrinsic agent. Major surgery is any invasive operative procedure in which a more extensive resection is performed (e.g. a body cavity is entered, organs are removed, normal anatomy is altered). In general, if a mesenchymal barrier is opened (pleural cavity, peritoneum, meninges), the surgery is considered major.

Senescence is the process of growing old which occurs in all species and is typified by a gradual slowing down of metabolism and breakdown of tissues, often accompanied by endocrinal changes.

Coronaviruses cause different coronavirus diseases including severe acute respiratory syndrome (SARS), Middle East respiratory syndrome (MERS) and coronavirus disease 2019 (COVID-19). Identified coronaviruses include SARS-CoV, MERS-CoV, SARS-CoV2, 229E, NL63, OC43, and HKU1.

Immune depression induced by sepsis, burn, trauma, major surgery, senescence and/or coronavirus showed important similarities, with for example induction of monocytosis and low HLA-DR on monocytes surface in trauma and sepsis (Heftrig et al., 2017, Mediat Inflamm, article ID 2608349, 12 pages), high production of pro-inflammatory cytokines (e.g. TNFα, IL-6 and IL-8) during senescence and after surgery or trauma (Jia et al., 2019, Eur J Trauma Emerg S, https://doi.org/10.1007/s00068-019-01271-6; Kovac et al., 2005, Exp Gerontol., 40(7):549-55), or persistently elevated IL-10 levels in the plasma of patients suffering from sepsis, burn injuries or trauma (Thomson et al., 2019, Military Medical Research, 6:11) and severe lymphopenia affecting all lymphocyte subsets, decreased mHLA-DR and moderately increased plasma cytokine levels showing at the same time both inflammatory (IL-6) and immunosuppressive (IL-10) responses for patients having trauma and coronavirus (Monneret et al., 2020, Intensive Care Med, https://doi.org/10.1007/s00134-020-06123-1).

More generally, immune depression induced by sepsis, burn, trauma, major surgery, senescence and/or coronavirus may be characterized by apoptosis of immune cells and/or high levels of anti-inflammatory cytokines that inhibit lymphocytes and macrophages and suppress the production of pro-inflammatory cytokines, and/or high levels of regulatory T-cells, and/or myeloid derived suppressor cells (MDSC), and/or inhibitory molecules (e.g. PD-1, PD-L1, CTLA4).

Immune depression may also result from secondary infections (e.g. nosocomial infections) occurring during immune depression induced by sepsis, burn, trauma, major surgery, senescence and/or coronavirus. Without limitation, said infections may be caused by:
Bacteria (e.g. Acinetobacter spp., Clostridium difficile, Escherichia coli, Klebsiella spp, Staphylococcus aureus, Pseudomonas aeruginosa, Mycobacterium tuberculosis, Enterococcus spp., Legionella spp.)
Viruses (e.g. Hepatitis B and C, influenza, HIV, rotavirus, herpes-simplex virus)
Fungi (e.g. Candida spp., Aspergillus spp., Fusarium spp., Mucorales spp., Scedosporium spp.)

Preferably, the invention provides a non-propagative viral vector encoding at least a polypeptide having an IL-7 activity or a composition comprising said viral vector for use in the treatment of immune depression induced by sepsis.

In another embodiment, the invention provides a non-propagative viral vector encoding at least a polypeptide having an IL-7 activity or a composition comprising said viral vector for use in the treatment of immune depression induced by SARS-CoV, MERS-CoV, SARS-CoV2, 229E, NL63, OC43, or HKU1.

In another embodiment, the invention provides a non-propagative viral vector encoding at least a polypeptide having an IL-7 activity or a composition comprising said viral vector for use in the treatment of immune depression induced by trauma (e.g. polytrauma, hip fracture), major surgery and senescence.

In one embodiment, the non-propagative viral vector or composition thereof is administered after the beginning of the immune depression phase induced by sepsis, burn, trauma, major surgery, senescence and/or coronavirus, within a period of time varying from 4 hours to 6 years. In one embodiment, the composition for use for the treatment of immune depression is administered at an early stage, preferably within the first month after the beginning of the immune depression phase, e.g. within 28 days, 21 days, 14 days, 7 days, 6 days, 5 days, 4 days, 3 days, 2 days, or even within the first 24 hours (e.g. 4, 6, 8, 12, 16, 20 hours) following the beginning of the immune depression phase. In another embodiment, the composition for use for the treatment of immune depression is administered within a period of time varying from approximately 1 month to approximately 6 months after the beginning of the immune depression phase (e.g. within 180 days, 150 days, 120 days, 100 days, 90 days, 80 days, 70 days, 60 days, 50 days, 40 days, 35 days, 30 days or 29 days). In still another embodiment, the composition for use for the treatment of immune depression is administered at a later stage after the beginning of the immune depression phase, preferably within 6 months to 6 years (e.g. 6 months, 7 months, 8 months, 9 months, 10 months, 11 months, 1 year, 2 years, 3 years, 4 years, 5 years, 6 years) after the beginning of the immune depression phase. One may also consider any combination of administrations of the composition thereof at different stages of the immune depression induced by sepsis, burn, trauma, major surgery, senescence, and/or coronavirus. For illustrative purposes, a suitable regimen may comprise one or more administrations at an early stage within the first month following the beginning of the immune depression phase, and one or more administrations at a later stage either within 1 month to 6 months, or within 6 months to 6 years after the beginning of the immune depression phase, or both within 1 month to 6 months or within 6 months to 6 years after the beginning of the immune depression phase.

Preferably, the non-propagative viral vector or composition thereof is administered after the beginning of the immune depression phase induced by sepsis within a period of time varying from 4 hours to 6 years. In one embodiment, the composition for use for the treatment of immune depression induced by sepsis is administered at an early stage, preferably within the first month after the beginning of the immune depression phase, e.g. within 28 days, 21 days, 14 days, 7 days, 6 days, 5 days, 4 days, 3 days, 2 days, or even within the first 24 hours (e.g. 4, 6, 8, 12, 16, 20 hours) following the beginning of the immune depression phase. In another embodiment, the composition for use for the treatment of immune depression induced by sepsis is administered within a period of time varying from approximately 1 month to approximately 6 months after the beginning of the immune depression phase (e.g. within 180 days, 150 days, 120 days, 100 days, 90 days, 80 days, 70 days, 60 days, 50 days, 40 days, 35 days, 30 days or 29 days). In still another embodiment, the composition for use for the treatment of immune depression induced by sepsis is administered at a later stage after the beginning of the immune depression phase, preferably within 6 months to 6 years (e.g. 6 months, 7 months, 8 months, 9 months, 10 months, 11 months, 1 year, 2 years, 3 years, 4 years, 5 years, 6 years) after the beginning of the immune depression phase. One may also consider any combination of administrations of the composition thereof at different stages of the immune depression induced by sepsis. For illustrative purposes, a suitable regimen may comprise one or more administrations at an early stage within the first month following the beginning of the immune depression phase, and one or more administrations at a later stage either within 1 month to 6 months, or within 6 months to 6 years after the beginning of the immune depression phase, or both within 1 month to 6 months or within 6 months to 6 years after the beginning of the immune depression phase induced by sepsis.

In a preferred embodiment, the use of non-propagative viral vector or composition comprises administration of said viral vector or composition between 1 and 10 times after the beginning of the immune depression phase, preferably between 1 and 5 times after the beginning of the immune depression phase, more preferably between 1 and 3 times after the beginning of the immune depression phase, even more preferably once after the beginning of the immune depression phase. In a more preferred embodiment, the one or more administration(s) are made by intravenous, subcutaneous or intramuscular administration(s) of the non-propagative viral vector or composition thereof.

The beneficial effects provided by the non-propagative viral vector or composition for use as described herein or the methods of the present invention can be evidenced by an observable improvement of the clinical status over the baseline status or over the expected status if not treated according to the modalities described herein. An improvement of the clinical status can be easily assessed by any relevant clinical measurement typically used by physicians and skilled healthcare staff and by techniques routinely used in laboratories. The clinical beneficial effects can also be evidenced by appropriate measurements such as blood tests, analysis of biological fluids, e.g. using various available antibodies to identify different immune cell populations involved in the immune response. Such measurements are evaluated routinely in medical laboratories and hospitals and a large number of kits are available commercially. They can be performed before the administration (baseline) and at various time points during treatment and after cessation of the treatment. In the context of the invention, said clinical beneficial effects can be associated with, but not limited to, the increase of the functional innate and/or adaptive immunity, the increase of the level of at least one type of cells associated with immunity, the increase of the level of at least one type of activated cells associated with immunity, and the immune homeostasis restoration (Takeyama et al., 2004, Critical Care 20048 (Suppl 1):P207). Cells associated with immunity comprise, but are not limited to CD4 T cells, CD8 T cells, B cells, NKT cells, NK cells, dendritic cells, monocytes, neutrophils, macrophages and eosinophils.

In the context of the invention, the therapeutic benefit can be transient (for one or a couple of days, weeks or months after cessation of administration) or sustained (for several months or years). As the natural course of clinical status which may vary considerably from a subject to another, it is not required that the therapeutic benefit be observed in each subject treated, but in a significant number of subjects (e.g. statistically significant differences between two groups can be determined by any statistical test known in the art, such as a Tukey parametric test, the Kruskal-Wallis test, the U test according to Mann and Whitney, the Student's t-test, the Wilcoxon test, etc.).

In one embodiment, a particularly appropriate use for the non-propagative viral vector or the composition described herein is for providing an increased concentration of a polypeptide having an IL-7 activity (e.g. hIL-7-Fc) in the blood circulation in the treated subject as compared to a subject not administered with said non-propagative viral vector or composition.

In a particular embodiment, the beneficial effects may be correlated with one or more of the followings:
increase of the functional innate and/or adaptive immunity in a subject administered with said non-propagative viral vector or composition compared to a subject not administered with said non-propagative viral vector or composition; and/or
increase of the level of at least one type of cells associated with immunity selected from the group consisting of CD4 T cells (e.g. naïve CD4 T cells, CD4+ effector memory cells, CD4+ central memory cells, CD4+ acute effector memory cells, etc.), CD8 T cells (e.g. naïve CD8 T cells, CD8+ effector memory cells, CD8+ central memory cells, CD8+ acute effector memory cells, etc.), B cells, NKT cells, NK cells, dendritic cells, monocytes and neutrophils in a subject administered with said non-propagative viral vector or composition compared to a subject not administered with said non-propagative viral vector or composition. Preferably, said use increases the level of preferably at least 2 types, more preferably of at least 3 types and more preferably at least 4 types of cells associated with immunity; and/or
increase of the activated status of at least one type of cells associated with immunity of at least one type (resulting in the increase of the number and/or the percentage of activated cells associated with immunity of at least one type) selected from the group consisting of activated CD4 T cells, activated CD8 T cells, activated B cells and activated NK cells in a subject administered with said non-propagative viral vector or composition compared to a subject not administered with said non-propagative viral vector or composition. Preferably, said use increases the activated status of preferably at least two types, more preferably of at least 3 types and more preferably at least 4 types of cells associated with immunity; and/or
increase of the level of cells in lungs, and more particularly of at least one type of cells associated with immunity selected from the group consisting of CD4 T cells, CD8 T cells and NK cells compared to a subject not administered with said non-propagative viral vector or composition, or administered with an IL-7 protein; and/or
increase, in lungs, of the activated status of at least one type of cells associated with immunity of at least one type (resulting in the increase of the number and/or the percentage of activated cells associated with immunity of at least one type) selected from the group consisting of activated CD4 T cells, activated CD8 T cells and activated NK cells in a subject administered with said non-propagative viral vector or composition compared to a subject not administered with said non-propagative viral vector or composition, or a subject treated with the IL-7 protein; and/or increase of the level of cytokines of at least one type selected from the group consisting of IFNγ, TNFα, IL-6 and IL-1β in a subject administered with said non-propagative viral vector or composition compared to a subject not administered with said non-propagative viral vector or composition; and/or
increase of Bcl2 gene expression on splenic and/or blood CD4 T cells, splenic and/or blood CD8 T cells and/or thymic cells in a subject administered with said non-propagative viral vector or composition compared to a subject not administered with said non-propagative viral vector or composition; and/or
decrease of the percentage of CD4+ and CD8+ double positive cells and increase of the percentage of CD4+ and CD8+ simple positive cells within the blood and/or spleen and/or thymus in a subject administered with said non-propagative viral vector or composition compared to a subject not administered with said non-propagative viral vector or composition; and/or
Increase of survival in a subject treated with said non-propagative viral vector or composition compared to a subject not administered with said non-propagative viral vector or composition, or a subject treated with the IL-7 protein.

In still another embodiment, the non-propagative viral vector or the composition as described herein, is for use in the treatment of immune-depressed subject displaying one or more biomarkers associated with the decrease of the level of cells associated with immunity and/or the level of activated cells associated with immunity. In a preferred embodiment, said one or more biomarkers is/are selected from the group consisting of HLA-DR expression on circulating monocytes, circulating IL-10, absolute CD3 T cell count, several immunosuppressive lymphocyte subpopulations measurements including regulatory T cells, and expression of inhibitory receptors like PD-1, PD-L1, CTLA-4, Lag-3, and BTLA.

In one embodiment, the non-propagative viral vector or composition described herein is for use for increasing the functional innate and/or adaptive immune system in a subject administered with said non-propagative viral vector or composition compared to a subject not administered with said non-propagative viral vector or composition. In a preferred embodiment, said non-propagative viral vector or composition is for use for increasing the level of at least one type of cells associated with innate immunity and/or the level of at least one type of activated or matured cells associated with innate immunity, and preferably selected from the group consisting of NK cells and monocytes. In another preferred embodiment, said non-propagative viral vector or composition is for use for increasing the level of at least one type of cells associated with adaptative immunity and/or the level of at least one type of activated cells associated with adaptative immunity, and preferably selected from the group consisting of CD4 T cells, CD8 T cells, B cells, and NK cells. For general guidance, the increase of the functional innate and/or adaptive immunity can be evaluated routinely, for example by analysis of biological fluids or by markers using suitable antibodies as described in the Example section.

The present invention also relates to a method for treating an immune depression induced by sepsis, burn, trauma, major surgery, senescence and/or coronavirus in a subject in need thereof comprising one or more administration(s) of a non-propagative viral vector or the composition described herein or prepared according to the process described herein.

Combination Therapy

In any of the embodiment of the invention, the non-propagative viral vector or composition thereof for use according to the present invention can be administered alone or in association with any conventional therapeutic modalities which are available for treating immune depression. For example, said viral vector or composition may be used in association with immunotherapeutic products. Representative examples include, among others, immune checkpoint modulators, agents that affect the regulation of cell surface receptors such as, e.g. monoclonal antibodies blocking Epidermal Growth Factor Receptor, monoclonal antibodies blocking Vascular Endothelial Growth Factor and TLR agonists (e.g. TLR9 agonists, TLR4 agonists). Other representative examples of such immunotherapeutic products suitable for use in the present invention are expression vectors (e.g. plasmid DNA vector, vaccinia virus, adenovirus, lentivirus, herpes virus, etc.) recombinant polypeptides and non-peptidic adjuvants, among many others.

All the above cited disclosures of patents, publications and database entries are specifically incorporated herein by reference in their entirety. Other features, objects, and advantages of the invention will be apparent from the description, drawings and from the claims. The following examples are incorporated to demonstrate preferred embodiments of the invention. However, in light of the present disclosure, those skilled in the art should appreciate that changes can be made in the specific embodiments that are disclosed without departing from the spirit and scope of the invention.

### EXAMPLES

1. Materials and Methods

The constructions described below are carried out according to the general genetic engineered and molecular cloning techniques detailed in Maniatis et al. (1989, Laboratory Manual, Cold Spring Harbor Laboratory Press, Cold Spring Harbor N.Y. or subsequent editions) or according to the manufacturer's recommendations when a commercial kit is used. PCR amplification techniques are known to the person skilled in the art (see for example PCR protocols—A guide to methods and applications, 1990, published by Innis, Gelfand, Sninsky and White, Academic Press).

1.1 Vector Construction

### 1.1.1 MVATG18897

MVATG18897, also named MVA-hIL-7-Fc, illustrated thereafter was engineered to express a human interleukin-7 fused to human Fc sequence of IgG2 (SEQ ID NO: 4) under the control of pH5R promoter.

A DNA fragment containing the pH5R promoter and the fusion IL-7-Fc surrounded by around 40 bp of sequences homologous to the vaccinia transfer plasmid was generated by synthetic way and inserted in plasmid 15ABXMTP_IL-7. After restriction of this plasmid by PsiI and ScaI, the fragment was inserted by In-Fusion cloning (In-Fusion HD cloning kit, Clontech) in the vaccinia transfer plasmid pTG18626 digested by NotI and BglII, resulting in pTG18897.

The MVA transfer plasmid, pTG18626, is designed to permit insertion of the nucleotide sequence to be transferred by homologous recombination in deletion 11 of the MVA genome. It originates from the plasmid pUC18 into which were cloned the flanking sequences (BRG3 and BRD3) surrounding the MVA deletion 11 (Sutter and Moss, 1992, Proc. Natl. Acad. Sci. USA 89:10847).

The homologous recombination was performed using a parental MVA containing gene encoding for the mCherry fluorescent protein into its deletion 11 (MVA mCherry). The advantage of MVA mCherry is to differentiate cells that are infected by the recombinant virus which have successfully integrated the expression cassette from the ones that are infected by the initial starting MVA mCherry virus (parental virus). Indeed, mCherry gene is removed in case of successful recombination of the expression cassette within deletion 11 and the viral plaques appear as white.

MVA-hIL-7-Fc (MVATG18897) was generated on chicken embryo fibroblast (CEF) by homologous recombination by using MVA-mCherry as parental virus and the transfer plasmid pTG18897. CEF were isolated from 12 day-old embryonated Specific pathogen free (SPF) eggs (Charles River). The embryos were mechanically dilacerated, solubilized in a Tryple Select solution (Invitrogen) and dissociated cells cultured in MBE (Eagle Based Medium; Gibco) supplemented with 5% FCS (Gibco) and 2 mM L-glutamine.

The homologous recombination between the transfer plasmid pTG18897 and the parental MVA-mCherry enables the generation of recombinant vaccinia viruses which have lost their mCherry expression cassette and gained the IL-7-Fc expression cassette and the selection was performed by isolation of white non-fluorescent plaques.

The viral stock of MVA-hIL-7-Fc (MVATG18897) was amplified on CEFs in two F175 flasks to generate appropriate stocks of virus. Viral stock was titrated on DF1 cells and infectious titers were expressed in pfu/mL. This stock was analyzed by PCR to verify the integrity of the expression cassettes and recombination arms using appropriate primer pairs. The stock was also analyzed by sequencing of expression cassette. Alignment of sequencing results showed 100% homology with the theoretical expected sequence. If needed, viral preparations were purified using conventional techniques. Briefly, viral amplification was performed at 37° C. 5% CO₂ for 72 h. Infected cells and medium were then pelleted and frozen. The crude harvest was disrupted using High Speed homogenizer (SILVERSON L4R) and submitted to a purification process (e.g. as described in WO2007/147528). Briefly, the lysed viral preparation can be clarified by filtration, and purified by a tangential flow filtration (TFF) step. Purified virus was resuspended in a suitable virus formulation buffer (e.g. 5% (w/v) Saccharose, 50 mM NaCl, 10 mM Tris/HCl, 10 mM Sodium Glutamate, pH8).

### 1.1.2 MVATG19791

MVATG19791 was engineered to express a human interleukin-7 fused to human Fc sequence of IgG2 (SEQ ID NO: 4) under the control of pH5R promoter. The nucleotide sequence encoding for the hIL-7-Fc was codon optimized to improve the expression of the recombinant protein in human cells. A Kozak sequence (ACC) was added before the ATG start codon and a transcriptional terminator (TTTTTNT) was added after the stop codon. Moreover, some patterns into the open reading frames were excluded: TTTTTNT, GGGGG, CCCCC which are deleterious for expression by poxvirus.

A DNA fragment containing the optimized fusion IL-7-Fc was generated by synthetic way. This fragment was inserted by In-Fusion cloning (In-Fusion HD cloning kit, Clontech) in the vaccinia transfer plasmid pTG19349 digested by PvuII, resulting in pTG19791.

The MVA transfer plasmid, pTG19349, is designed to permit insertion of the nucleotide sequence to be transferred by homologous recombination in deletion 11 of the MVA genome. It originates from the plasmid pUC18 into which were cloned the flanking sequences (BRG3 and BRD3) surrounding the MVA deletion III. It contains also the pH5R promoter.

MVATG19791 was generated on chicken embryo fibroblast (CEF) by homologous recombination by using MVA-mCherry as parental virus and the transfer plasmid pTG19791 as described above.

1.2. In Vitro Assessment of MVA-hIL-7-Fc (MVATG18897 and MVATG19791)

As a first step, ability of MVA-hIL-7-Fc (MVATG18897) to express the hIL-7-Fc gene was assessed in A549 cells infected with the viral vector. Cells and supernatants were collected and submitted to Western Blot, and hIL-7-Fc detection was assessed using a rabbit monoclonal antibody specific of human IL-7. It was compared to cells transduced with MVA empty (MVATGN33.1) which do not encode any foreign protein.

Ability of MVA-hIL-7-Fc (MVATG18897) to express functional hIL-7 was also assessed. COS7 cells were infected at MOI (multiplicity of infection) 0.3, 1 and 3, and supernatants were collected 48 to 72 hours later. Quantity of secreted IL-7 was assessed by an ELISA and IL-7 functionality was evaluated using PB1 cells (ATCC), which proliferation is dependent of hIL-7. Supernatants were incubated for 72 h on PB1 cells and an MTT assay aiming at measuring cellular metabolism (reflecting cell proliferation) was performed.

As a second step, ability of MVATG18897 and MVATG19791 to express the hIL-7-Fc gene was assessed and compared in CEF and A549 cells infected with the viral vector. Cells and supernatants were collected and submitted to Western Blot, and hIL-7-Fc detection was assessed using a rabbit monoclonal antibody specific of human IL-7. It was compared to cells transduced with MVA empty (MVATGN33.1) which do not encode any foreign protein. Amount of hIL-7-Fc in supernatants was also quantified using hIL-7 ELISA.

1.3. Pharmacokinetics and Biological Activities in a Healthy C57BL6/J Mice Model

1.3.1 Animals

The pharmacokinetics of human Interleukin-7 fused to a Fc domain of a human IgG2 (hIL-7-Fc) expressed by the MVA-hIL-7-Fc (MVATG18897) was assessed following administration in C57BL/6J mice purchased at Charles River Laboratories.

1.3.2. Administration Protocols

1.3.2.1 MVA-hIL-7-Fc (MVATG18897) and MVA Empty (MVATGN33.1) Injection Protocol

Experiment 1: Mice were injected once intravenously (IV) at the retro-orbital sinus with 1×10⁷ or 1×10⁸ pfu of MVA-hIL-7-Fc (MVATG18897) or 1×10⁸ pfu of MVATGN33.1 (empty MVA used as control) (15 mice/group).

Experiment 2: Mice were injected once intravenously (IV) at the retro-orbital sinus with 1×10⁸ pfu of MVA-hIL-7-Fc (MVATG18897) or 2×10⁸ pfu of empty MVA (MVATGN33.1) (15 mice/group).

Experiment 3: Mice were injected once intravenously (IV) at the retro-orbital sinus with 1×10⁸ pfu of MVA-hIL-7-Fc (MVATG18897) or 1×10⁸ pfu of empty MVA (MVATGN33.1). The experiment was performed twice, and results were pooled.

Experiment 4: Mice were injected once intravenously IV) at the retro-orbital sinus with 1×10⁷ pfu of MVA-hIL-7-Fc (MVATG18897) or 5 μg of a recombinant IL-7-Fc protein produced on CHO cells by Geneart.

1.3.3 Pharmacokinetics of hIL-7-Fc and of mIFNγ

1.3.3.1 Blood Sampling

Experiment 1: In order to measure the circulating hIL-7-Fc and mIFNγ, blood of injected mice was sampled at the following time-points: 0 h, 2 h, 6 h, 24 h, 48 h, 72 h, 96 h, 8, 15 and 21 days (3 mice per group at each timepoint).

Experiment 2: In order to measure the circulating hIL-7-Fc produced by MVA-hIL-7-Fc (MVATG18897) injection and the amount of circulating mIFNγ, blood of injected mice was sampled at the following time-points: 0 h, 6 h, 24 h, 48 h, 72 h, 96 h, 7 and 9 days (3 mice per group at each timepoint). 1.3.3.2 Serum Preparation

Blood samples were kept for about 1 h at 4° C. after sampling. Then they were centrifugated (10000 rpm for 5 min at 4° C.) and sera were collected in new tubes and stored at −20° C. upon ELISA.

1.3.3.3 hIL-7 and mIFN-Gamma ELISA

For the hIL-7-Fc pharmacokinetics, the ELISA was run using the human IL-7 Duoset ELISA from R&D systems according to provider instructions. Of note, the volume of samples and various reagents used was only of 50 μL all along the assay. Samples were diluted from 1/3 to 1/6561 depending on samples and IL-7 concentrations. The concentration of each sample was then calculated using a standard curve established and provided by R&D System, encompassing values from 7.81 pg/mL to 500 pg/mL.

For the dosing of mIFNγ, the ELISA was run using the mouse IFN-gamma ELISA MAX™ Deluxe from Biolegend according to provider instructions. Of note, except for the coating step, the volume of samples and various reagents used was only of 50 μL all along the assay. Samples were diluted from 1/3 to 1/6561 depending on samples and IFN-gamma concentrations. The concentration of each sample was then calculated using a standard curve established and provided by Biolegend, encompassing values from 15.62 pg/mL to 1000 pg/mL.

1.3.4 Biological Activity Assessment

Biological activity was not assessed in experiment 1.

Experiment 2: To measure the biological activity of MVA-hIL-7-Fc (MVATG18897), 3 mice per group were sacrificed at days 1, 3, 9 and 29, and spleens and thymus were collected.

Spleen cells and thymic cells were prepared, and red blood cells were lysed. After washings, collected cells were stained using the following antibodies and reagents: Fc blocker solution (Anti CD16-32 (2.4G2)), anti-CD3 APC, anti-CD4 APC-H7, anti-CD8 PE-CY7 or anti-CD8-PRCP, anti-CD19-PE, anti-B220-APCH7 (, anti-NK1.1 PercpCy-5, anti-CD11c-PE, anti-CD11b-V500, anti-Ly6G-FITC, Ly6C-APC, anti-Bcl2-FITC, anti-CD62L-PECy7, anti-CD44-FITC, anti-CD127-PE, viability marker (LiveDead LV450), all these reagents being purchased at Becton Dickinson, except viability marker from Invitrogen. The incubation with Fc blocker solution was done prior to incubation with antibodies. Cells were then incubated with 3 different mixes/panels of antibodies:
T cells: anti-CD3, anti-CD8, anti-CD4, anti-CD44, anti-CD62L, LV450
Myeloid cells: anti-B220, anti-NK1.1, anti-CD11b, anti-CD11c, anti-Ly6G, anti-Ly6C, LV450
Apoptosis: for spleen, anti-CD4, anti-CD8, anti-CD3, anti-Bcl2, LV450; for thymus, same antibody mix except anti-CD3

Washed stained samples were run on a FACSCanto II cytometer. Data were analyzed using the BD diva software.

Experiment 3 (performed twice and pooled): to measure biological activities of MVATG18897 and MVATGN33.1 on lungs, 5 mice per group were sacrificed at day 3 and day 9 post injection and lungs were collected.

Lung cells were prepared, and red blood cells were lysed. After washings, collected cells were counted using the Muse Cell Analyzer and stained using the following antibodies and reagents: Fc blocker solution (Anti CD16-32 (2.4G2)), anti-CD3 APC, anti-CD4 APC-H7, anti-CD8 PE-CY7, anti-NK1.1 PercpCy-5, anti-CD69-FITC, viability marker (LiveDead LV450), all these reagents being purchased at Becton Dickinson, except viability marker from Invitrogen. The incubation with Fc blocker solution was done prior to incubation with antibodies. Stained were analyzed by flow cytometry (FACSCanto II).

Experiment 4 (performed twice, one for spleen analyses, one for lung analyses): to measure biological activity of MVATG18897 and hIL-7-Fc protein, 6 mice per group were sacrificed at day 3 and 9 post injection in each experiment, and spleens or lungs were collected.

Spleen cells and lung cells were prepared, and red blood cells were lysed. After washings, collected cells were counted using the Muse Cell Analyzer and stained using the following antibodies and reagents: Fc blocker solution (Anti CD16-32 (2.4G2)), anti-CD3 APC, anti-CD4 APC-H7, anti-CD8 PE-CY7, anti-NK1.1 PercpCy-5, anti-CD69-FITC, viability marker (LiveDead LV450), all these reagents being purchased at Becton Dickinson, except viability marker from Invitrogen. The incubation with Fc blocker solution was done prior to incubation with antibodies. Stained were analyzed by flow cytometry (FACSCanto II).

An intracellular cytokine staining assay was also performed using spleen and lung cells. Cells were stimulated for 4 h30 with anti-CD3 (final dilution 1/1000) and anti-CD28 (final dilution 1/1000) antibodies, at 1 μg/mL each, in the presence of Golgi Plug (final dilution 1/1000). After incubation, cells were stained using the following antibodies and reagents: Fc blocker solution (CD16/32), anti-CD4-APC-H7, anti-CD8-V500, viability marker (Live/Dead LV450). The incubation with Fc blocker solution was done prior to incubation with antibodies. Then, cells were fixed and permeabilized and intracellular cytokine staining was performed with the following antibodies: anti-CD3-PerCP, anti-IFNγ-Alexa488, anti-TNFα-APC and anti-IL2-PE. Samples were run on a BD Biosciences FACSCanto II cytometer. Data were analyzed using the BD Biosciences Diva software.

1.3.5 Statistical Analyses

For assessment of biological activity, statistical analyses (GraphPad Prism software) were performed using the two-way ANOVA with repeated measures (mixed model) test followed if significant (p<0.05) by Bonferonni post-tests to compare group by group. The p values were corrected due to multiple comparisons. The difference observed between two groups was considered as significant if the p-value after the Bonferonni test and after correction was lower than 0.05.

1.4. Biological Activities in a Sepsis Mouse Model

1.4.1. Cecal Ligation and Puncture (CLP)

To induce polymicrobial peritonitis, 8 to 11 weeks old C57BL/6 male mice were subjected to cecal ligation and puncture (CLP) surgery adapted from the protocol described by Restagno et al. (2016, PLoS One, 11(8):e0162109). Briefly, mice were anesthetized with a mix of xylazine and ketamine. Cecum was exposed, ligated at its external third and punctured twice with a 21-gauge needle to create two single holes. A small amount of feces was extruded in order to induce a mild grade sepsis. Controls were naive mice and Sham-operated mice undergoing laparotomy with only exposition of cecum without CLP. All operated mice received analgesia (buprenorphine) prior to surgery. At the end of the surgery, all operated mice received a subcutaneous injection of 5% glucose saline solution.

Approximately six hours following surgery and then every twelve hours for the next two days, all operated mice received an intraperitoneal injection of antibiotic (imipenem/cilastatin) and a subcutaneous injection of buprenorphine to manage pain. Mice were then monitored twice a day until the end of the study. Clinical score is defined, if the score is above the pre-determined threshold, mice were euthanized. Inability to rise, labored breathing, anorexia or body temperature below 30° C., was observed, the animal was euthanized. Survival rates were then determined until day 7 post-surgery.

1.4.2. Administration Protocols

Experiments comparing Sham, untreated CLP and MVA-hIL-7-Fc treated mice: 4 days after CLP, one group of CLP mice was injected once intravenously (IV) at the retro-orbital sinus with 1×10⁸ pfu of MVATG18897 (MVA-hIL-7-Fc) in 100 μL of S08 buffer after anesthesia with isoflurane inhalation.

Experiment comparing MVA-empty and MVA-hIL-7-Fc treated CLP mice: 4 days after CLP, one group of CLP mice was injected once intravenously (IV) at the retro-orbital sinus with 1×10⁸ pfu of MVATG18897 (MVA-hIL-7-Fc) in 100 μL of S08 buffer after anesthesia with isoflurane inhalation. The 2^{nd} group was treated exactly the same way but with MVA-empty.

Experiments comparing MVA-hIL-7-Fc and hIL-7-Fc protein (2 experiments): 4 days after CLP, one group of CLP mice was injected once intravenously (IV) at the retro-orbital sinus with 1×10⁷ pfu of MVATG18897 (MVA-hIL-7-Fc) in 100 μL of S08 buffer after anesthesia with isoflurane inhalation. The 2^{nd} group was treated exactly the same way but with 5 μg of hIL-7-Fc.

1.4.3. Biological Activity Assessment

1.4.3.1. Blood

1.4.3.1.1. Serum Preparation

At the end of the study, blood was collected into collection tubes (Microvette® CB 200 Z-Gel, Sarstedt). Blood samples were kept for about 1 h at 4° C. after sampling. Then, they were centrifugated (10000 rpm for 5 min at 4° C.) and sera were collected in new tubes and stored at −20° C. until ELISA and Multiplex assay.

1.4.3.1.2. Cell Suspension Preparation

Blood was taken from retro-orbital sinus. A small volume of whole blood was lysed with 1% acetic acid solution, mixed and acquired during a fixed time at medium velocity (1 μL/sec) of BD FACSCanto II. The number of events corresponding to non-lysed nuclei was counted, thus giving concentration of nucleated cells per μL of whole blood. For further flow cytometry analysis, remaining whole blood was lysed with 10 mL of Red Blood Lysis Buffer (BioLegend) for approx. 15 min. After washing, cells were proceeded for staining as for spleen cells.

1.4.3.2. Spleen

To measure the biological activity of MVA-hIL-7-Fc (MVATG18897), mice were sacrificed three days after MVA-hIL-7-Fc injection, i.e. seven days after CLP, and spleens were collected. Spleens were dissociated and cell suspensions were obtained. After lysis of red blood cells, spleen cells were collected, numerated and proceeded for immune assays described below.

1.4.3.3. hIL-7 ELISA

The ELISA was run using the human IL-7 DuoSet® ELISA development system from R&D Systems according to provider instructions. Of note, all reagent volumes were divided by two. Sera were diluted at 1/3 and then by 5 consecutive three-fold dilutions. The concentration of each sample was then calculated using a standard curve established and provided by R&D Systems, encompassing values from 7.81 pg/mL to 500 pg/mL.

1.4.3.4. Multiplex Assay

The assay was performed on both sera and supernatants of stimulated splenocytes. Splenocytes were seeded at 0.5×10⁶ cells per well pre-coated with an anti-CD3 antibody (1 μg/mL). Twenty-four hours later, supernatants were collected and stored at −20° C. The multiplex assay was run on sera and supernatants using the U-PLEX Multiplex assay from MSD (Meso Scale Discovery) according to provider instructions in order to assess the following cytokines: IL-1β, IL-6, IL-10, TNFα and IFNγ. The concentration of each sample was then calculated using a standard curve established and provided by MSD.

1.4.3.5. IFNγ ELISpot Assay

Splenocytes were plated at 25×10³ cells per well and cultured overnight (approx. 20 h) with an anti-CD3 antibody (1 μg/mL) and an anti-CD28 antibody (1 μg/mL). IFNγ-producing T cells were quantified by murine IFNγ single-color enzymatic ELISpot (enzyme linked immunospot) assay from CTL (MIFNGP-1M/5). The number of spots (corresponding to the IFNγ-producing T cells) in negative control wells were subtracted from the number of spots detected in experimental wells containing anti-CD3/CD28 antibodies.

1.4.3.6. Flow Cytometry Analysis

1.4.3.6.1. Cell Phenotyping

Splenocytes were stained using the following antibodies and reagents: Fc blocker solution (Anti-CD16/32, clone 2.4G2), anti-CD45-PE-Cy7 anti-CD3 APC (anti-CD4 APC-H7, anti-CD8 V500, anti-CD19-PE, anti-CD69-FITC, anti-NK1.1 PercpCy-5, viability marker (LiveDead LV450), all reagents being purchased at Becton Dickinson, except viability marker from Invitrogen. The incubation with Fc blocker solution was done prior to incubation with antibodies. Samples were then run on a FACSCanto II cytometer and data were analyzed using the BD Diva software.

1.4.3.6.2. Triple Intracellular Cytokine Staining Assay

Splenocytes were stimulated for 4 hours with an anti-CD3 antibody (1 μg/mL) and an anti-CD28 antibody (1 μg/mL) in the presence of Golgi Plug (1/1000). Cells were then stained using the following antibodies and reagents: Fc blocker solution (anti-CD16/32, clone 2.4G2), anti-CD4 V500, anti-CD8 APC-H7, viability marker (LiveDead LV450). The incubation with Fc blocker solution was done prior to incubation with antibodies. After washings, cells were fixed and permeabilized. Intracellular cytokine staining was performed with the following antibodies: anti-CD3 PerCP, anti-IFNγ Alexa488, anti-TNFα APC and anti-IL2 PE. Samples were then washed and run on a FACSCanto II cytometer. Data were analyzed using the BD Diva software.

1.4.3.7. Statistical Analyses

For survival study, statistical analyses (SAS® 9.4 software) were performed using log-rank test followed by ad-hoc comparisons between groups using Tukey multiplicity adjustment test.

For assessment of biological activity in experiments comparing Sham, untreated CLP and MVA-hIL-7-Fc treated mice and experiment and comparing MVA-empty and MVA-hIL-7-Fc treated CLP mice, statistical analyses (GraphPad Prism software) were performed using the one-way ANOVA Kruskal-Wallis test followed if significant (p<0.05) by pairwise Mann-Whitney tests to compare group by group. The difference observed between two groups was considered as significant if the p-value obtained with the Mann-Whitney test was lower than 0.05.

For assessment of biological activity in experiments comparing MVA-hIL-7-Fc and hIL-7-Fc protein statistical analyses (GraphPad Prism software) were performed using the one-way ANOVA Kruskal-Wallis test followed if significant (p<0.05) by pairwise Dunns tests to compare group by group. The difference observed between two groups was considered as significant if the p-value obtained with the Dunns test was lower than 0.05.

1.5. Biological Activities in ICU Covid-19 Immuno-Suppressed Patients

Whole blood was obtained from SARS-COV-2 infected patients enrolled in the observational clinical study RICO (REA-IMMUNO-COVID) approved by ethics committee (Comité de Protection des Personnes Ile de France 1—N^{o}IRB/IORG #: IORG0009918—agreement 2020-A01079-30) and registered at ClinicalTrials.gov under NCT04392401 number (intensive care units of Hospices civils de Lyon). Samples from six patients displaying a SOFA score > or equal to 5 at admission were obtained between day 3 and 5 after ICU entry.

STAT5 phosphorylation after stimulation with supernatants containing hIL-7-Fc was assessed. Whole blood was stimulated ten minutes using diluted supernatants from MVATG18897 or MVATGN33.1 infection (1/2.5, 1/5 and 1/10 dilutions) and non-infected cells supernatant (1/2.5 dilution). Cells were stained with anti-human CD3 Pe-Cy7 and anti-human CD45 APC-H7. Fixation, red blood cell lysis and permeabilization steps were performed using Perfix-Expose kit. After permeabilization, cells were stained with anti-human pSTAT5 (pY694). Cells were analyzed on Cantoll flow cytometer and data were analyzed using the Flow Jo software.

Functionality of CD4+ T cells was determined by intracellular staining of cytokines. Whole blood was stimulated using Duractive 1 stimulation kit according to manufacturer's instruction in addition to supernatant from MVATG18897 or MVATGN33.1 infection or non-infected cells supernatant. Supernatants were diluted at 1/2 and 1/10 to stimulate cells. Intracellular staining of CD4+ T cells was performed using Duraclone IF T activation kit according to manufacturer's instruction. Cells were analyzed on Cantoll flow cytometer and data were analyzed using the Flow Jo software.

1.6. Biological Activities in Senescent Trauma Patients

A prospective observational clinical study HIPAGE including hip fracture patients, aged over 75 years admitted in emergency department of Pitie Salpetriere hospital was approved by the ethics committee (CPP Pitie-Salpetriere, Paris, France). In addition, age matched senescent control patients were also recruited. All participants included were informed and gave their consent. Analyses were performed on cryopreserved PBMCs of 5 senescent patients and 10 senescent hip fractured patients obtained 48 to 72 hours after surgery.

Immunophenotyping was carried out to assess the expression of CD57 among CD8 T cells, a well-known immunosenescent marker (Kared et al., 2016, J Immunol Immunother, 65(4):441-52). PBMCs were thawed and stained with Fixable Viability Dye (eFluor 780), anti-human CD3-Pe-Cy7, anti-human CD8-BV650 and anti-human CD57-FITC. BD-LSRFortessa flow cytometer was used to analyzed cells and data were analyzed using the Flow Jo software.

STAT5 phosphorylation after stimulation with supernatants containing hIL7-Fc was assessed on thawed PBMCs. PBMCs were stimulated for ten minutes using diluted supernatants from MVATG19791 infected cells (at a dilution corresponding to 12.5 ng/mL of IL-7-Fc) or from MVATGN33.1 infected cells (with the same dilution). Unstimulated controls with culture medium were simultaneously used. Fixation was performed using BD-Cytofix Buffer, and permeabilization was carried out using BD-Perm Buffer III. After permeabilization, cells were stained with anti-human CD3 Pacific Orange, anti-human pSTAT5 (pY694). BD-LSRFortessa flow cytometer was used to analyze cells and data were analyzed using the Flow Jo software.

T cell proliferation was evaluated on thawed PBMCs. PBMCs were stained with Cell Proliferation Dye (eFluor 450) and stimulated for five days using diluted supernatants from MVATG19791 infected cells (at a dilution corresponding to 12.5 ng/mL of IL7-Fc) or from MVATGN33.1 infected cells (with the same dilution). Unstimulated controls with culture medium were simultaneously used. Then, PBMCs were stained with Fixable Viability Dye (eFluor 780) and anti-human CD3-Pe-Cy7. Cells were analyzed on BD-LSRFortessa flow cytometer and data were analyzed using the Flow Jo software.

1.7. Biological Activities in ICU Trauma and Heavy Surgery Patients

Cryopreserved PBMCs were obtain form a cohort of critically ill patients admitted in intensive care units after a traumatic chock or after a heavy surgery procedure. Blood from patients with a SOFA score higher or equal to 4 at the admission were obtained at day 3 to 5 after ICU admission. PBMC were immediately prepared and frozen till analysis. PBMCs were provided by Transhit Biomarker company, with a guaranty of ethical conditions including consent of patients and/or from the surrounding family. Three patients after a heavy surgery procedure, and three patients after a strong polytraumatic chock were analyzed.

Functionality of CD4+ T cells was determined by intracellular staining of cytokines on PBMC of 3 trauma and 3 surgery patients. After thawing and overnight resting, PBMCs were stimulated in presence of protein transport inhibitor (Brefeldin A (GolgiPlug BD)) for 5 hours using PMA 3 ng/mL-ionomycin 0.3 μg/mL, or anti-CD3/anti-CD28 antibodies (1 μg/mL each). Unstimulated controls were simultaneously used. For each condition of stimulation, supernatant from MVATG19791 (at a dilution corresponding to 60 ng/mL of IL7-Fc) or from MVATGN33.1 (with the same dilution) infected cells was added and compared to controls with culture medium only.

Then, PBMC were fixed and permeabilized (BD cytofix/cytoperm), and stained with monoclonal antibodies against CD3-PECy7, CD4-BV510, CD8-APC-H7, IFNγ-BB700, IL2-PE, TNFα-A488. Cells were analyzed on Cantoll flow cytometer and data were analyzed using the Flow Jo software.

2. Results

2.1 In Vitro Assessment of hIL-7-Fc Expression and Functionality Following Cell Transduction by MVA-hIL-7-Fc (MVATG18897)

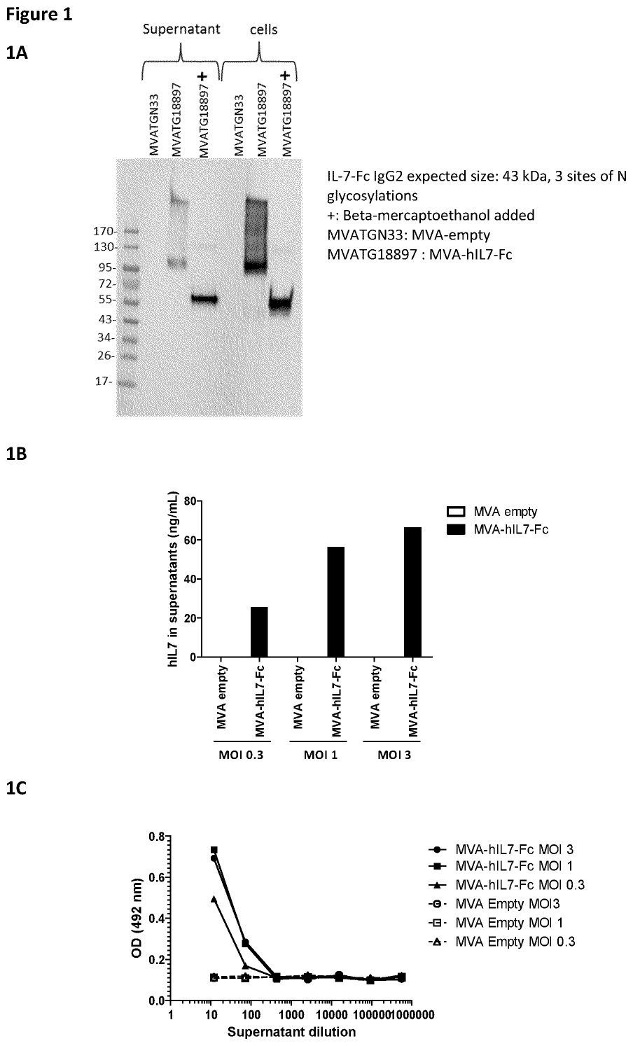

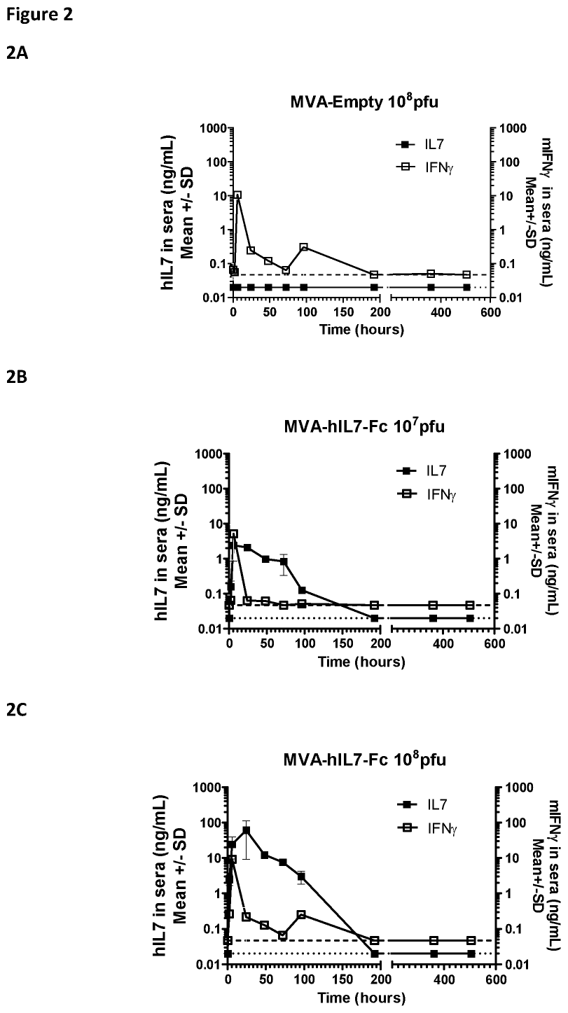

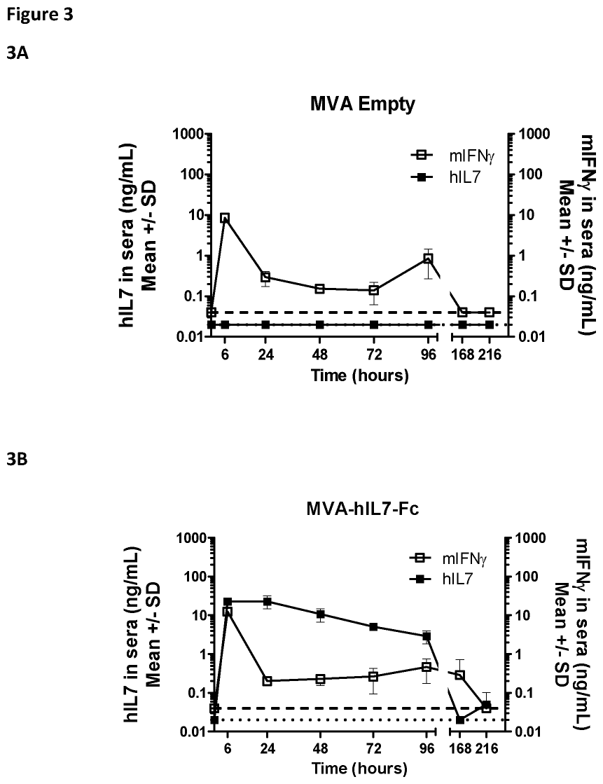

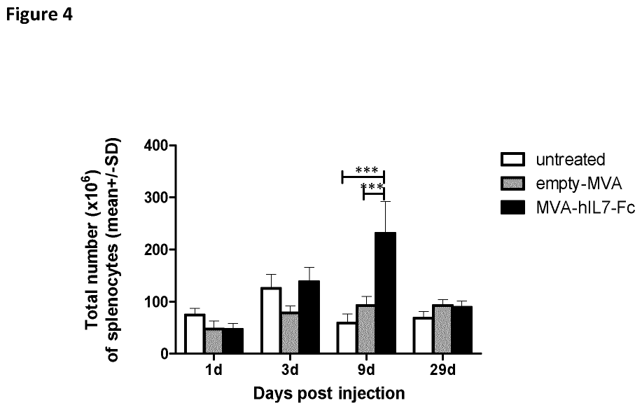

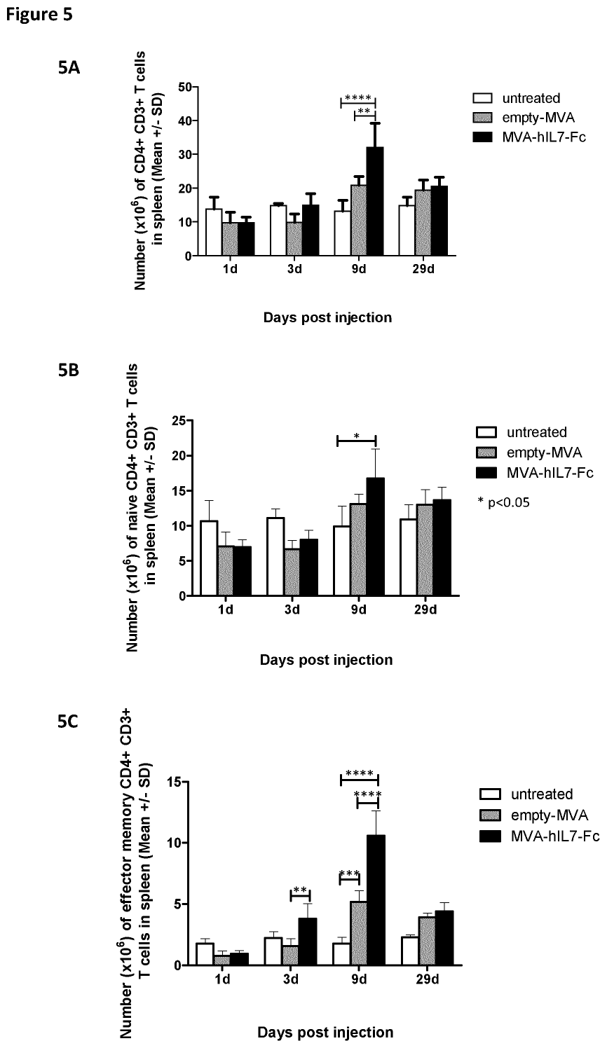

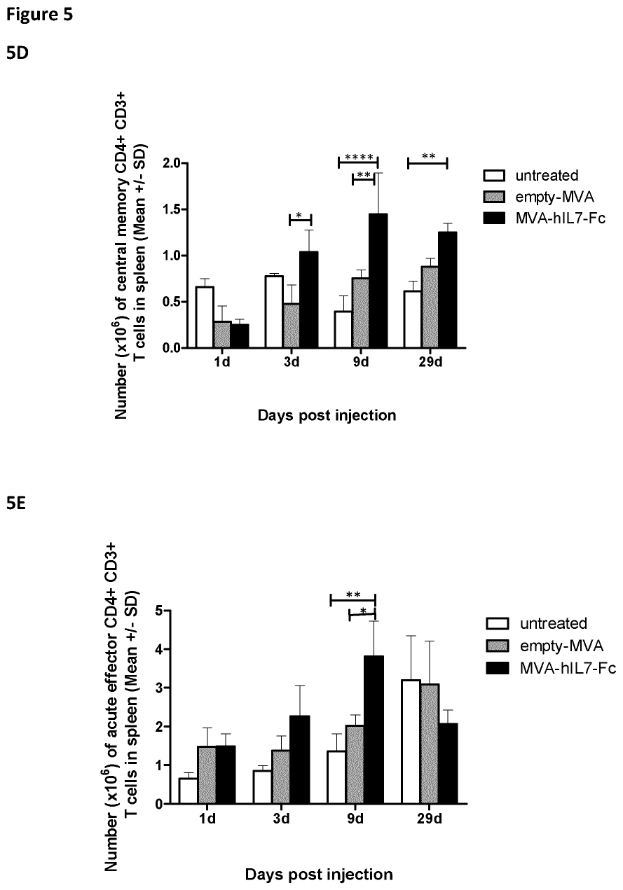

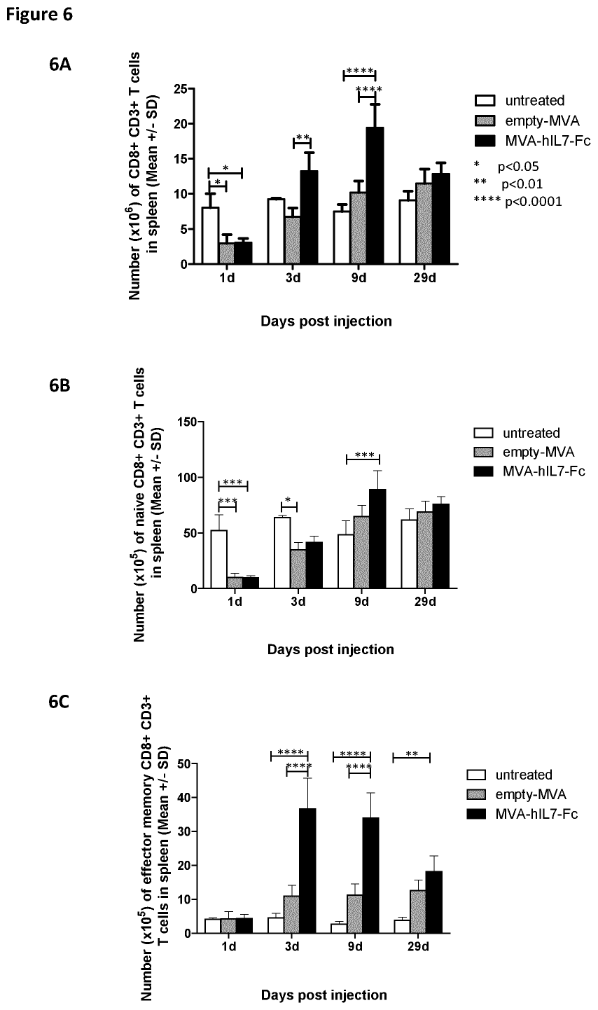

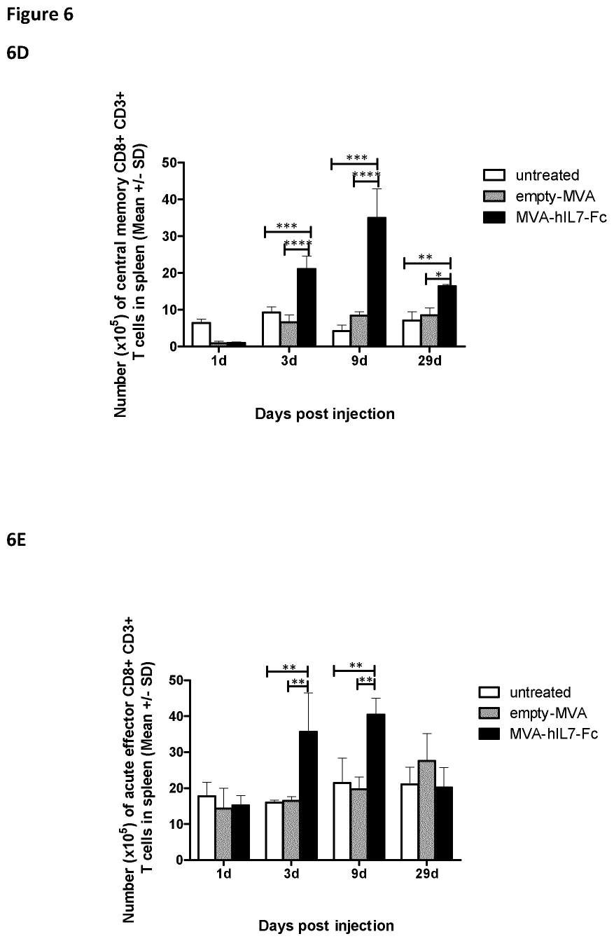

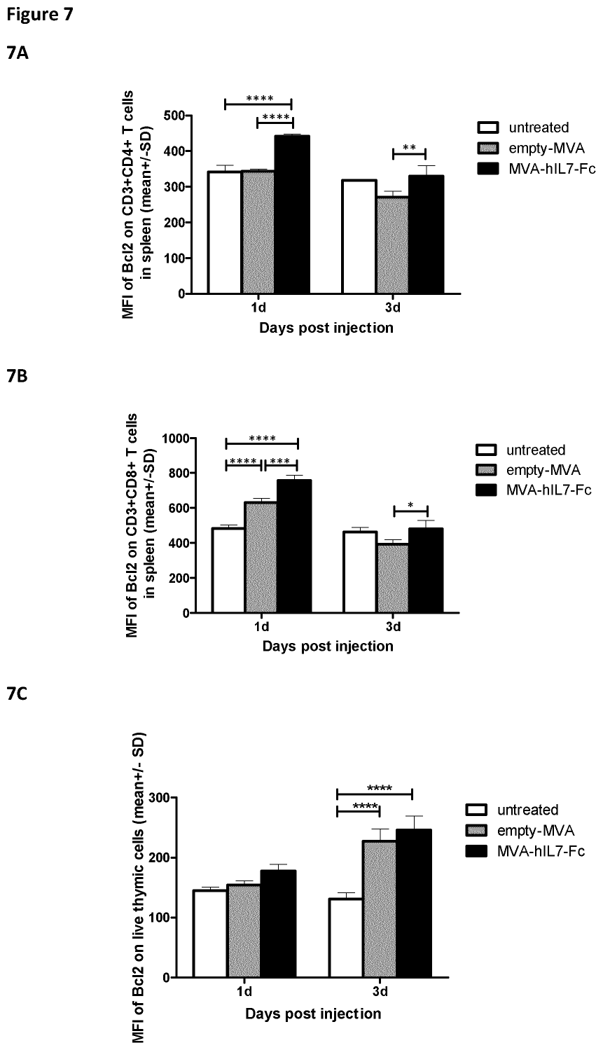

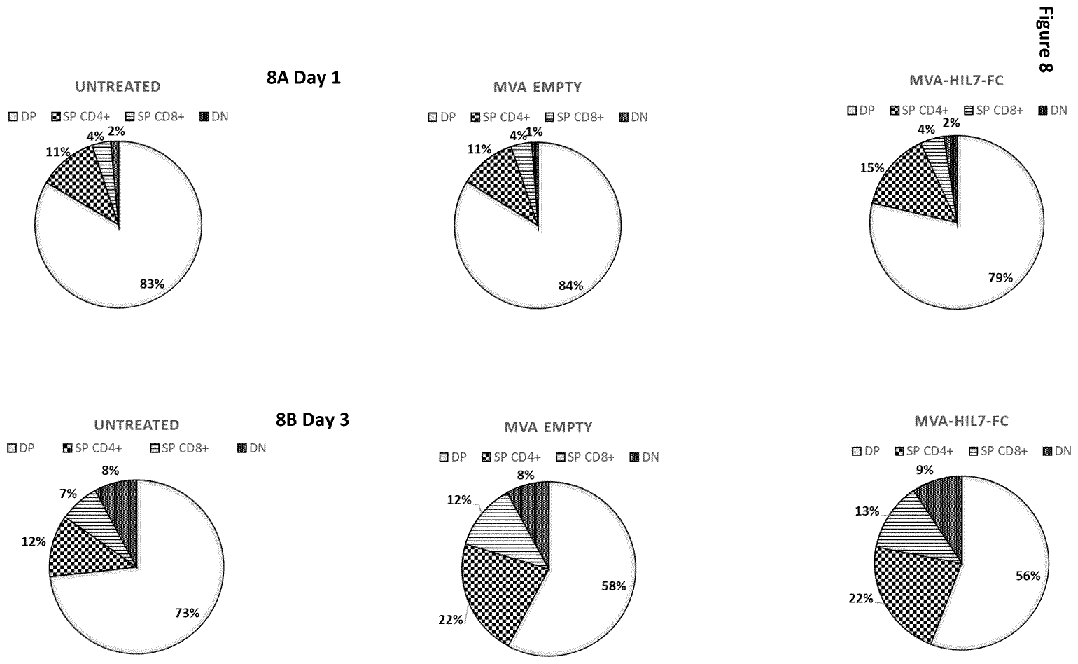

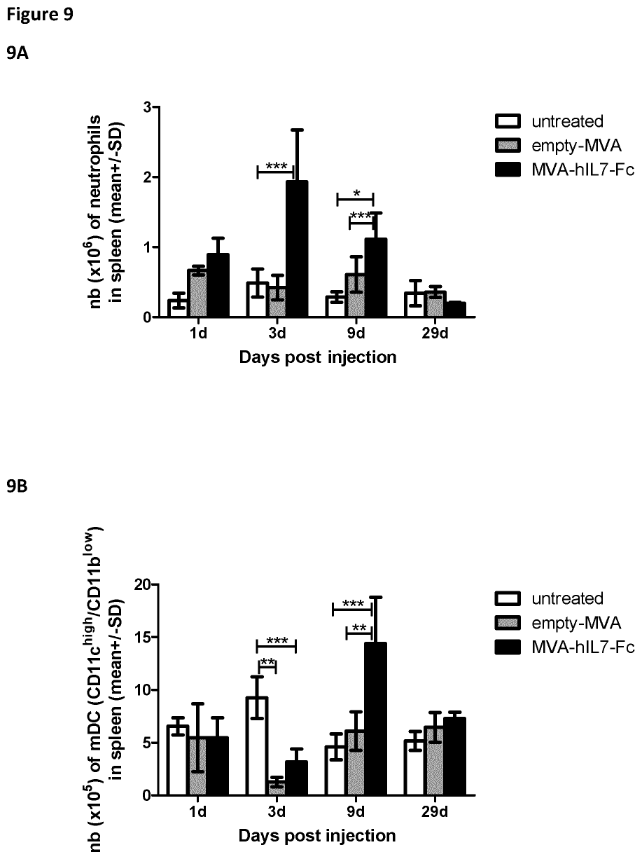

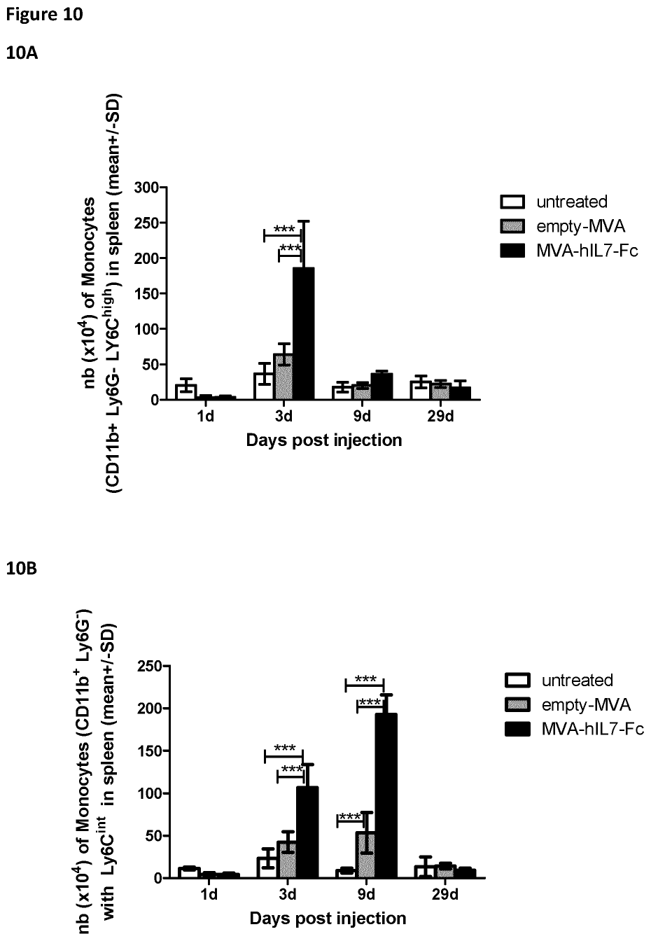

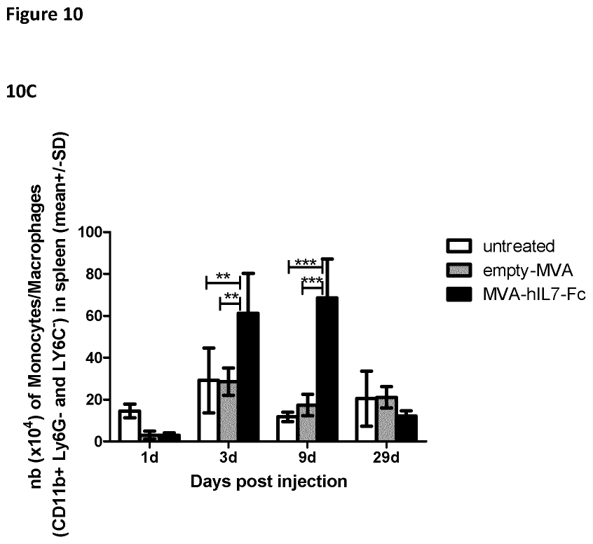

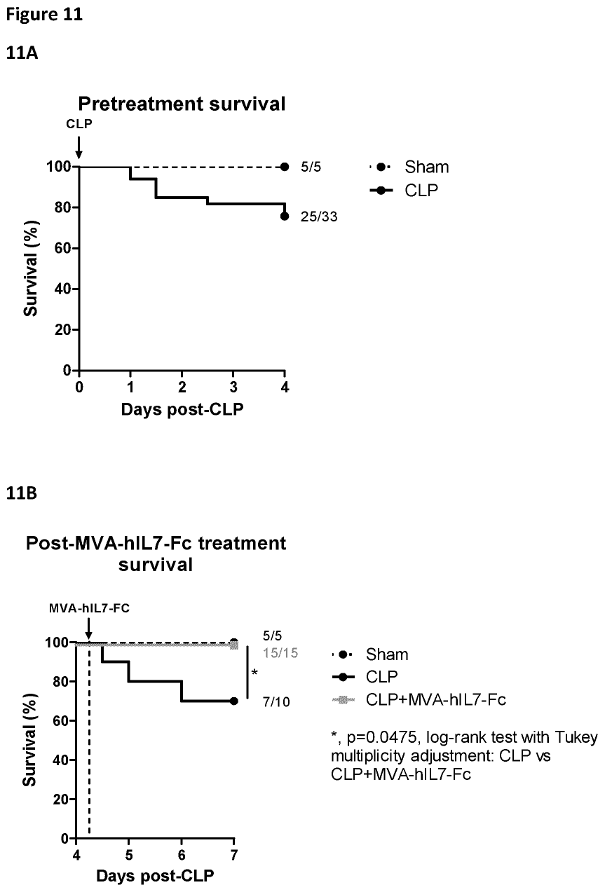

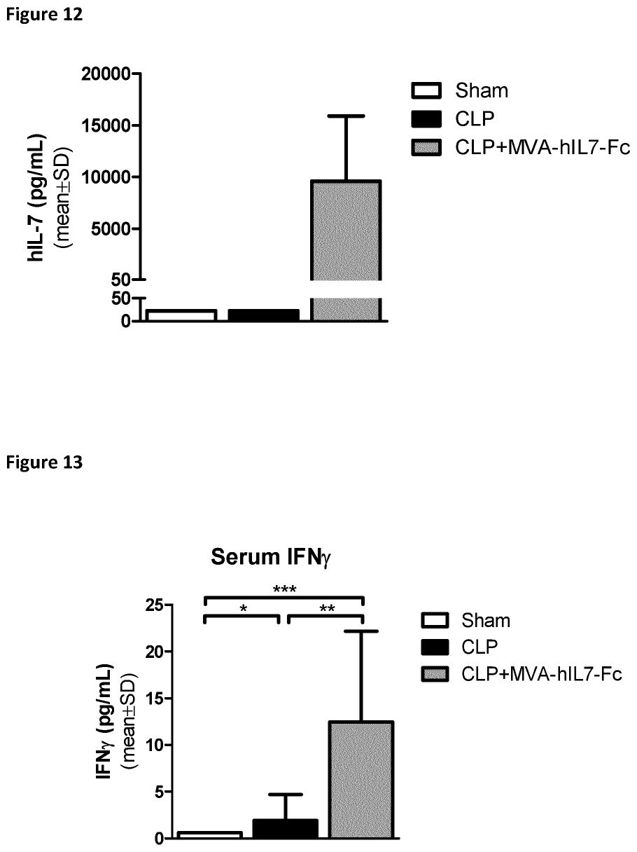

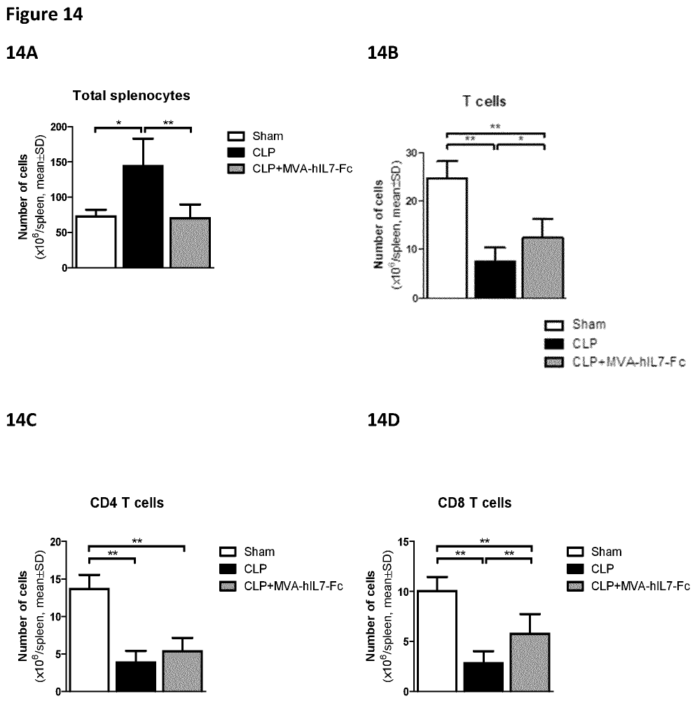

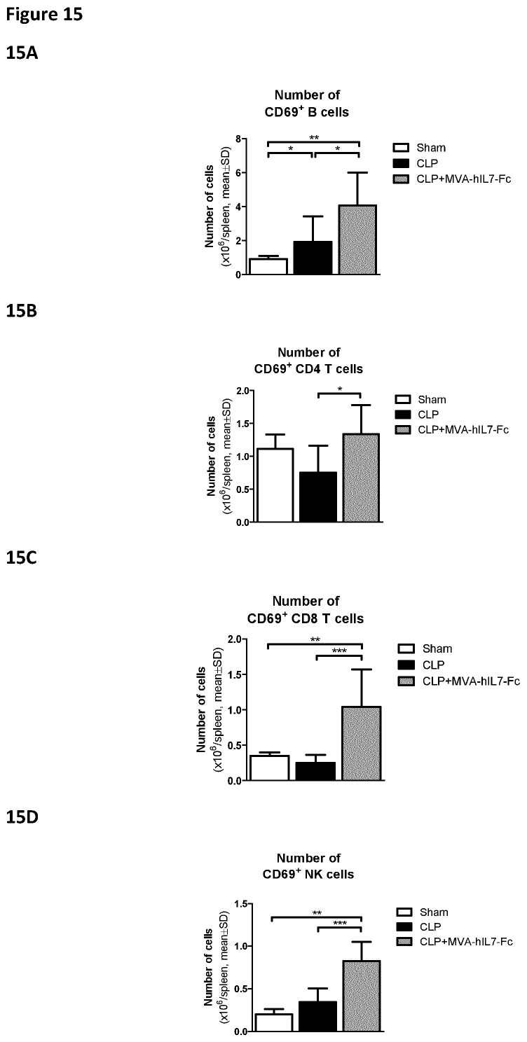

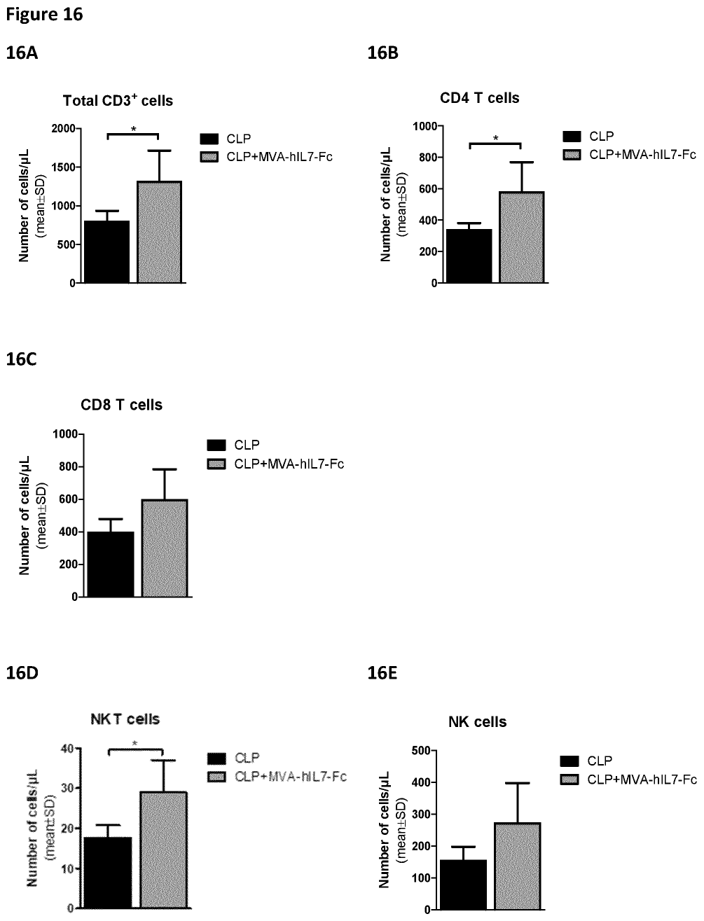

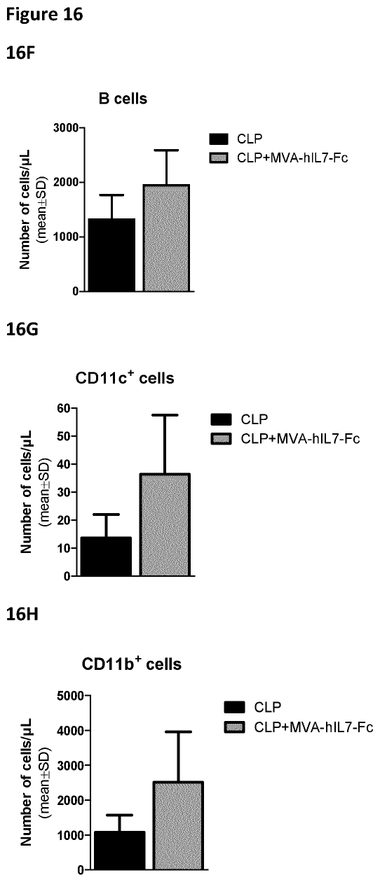

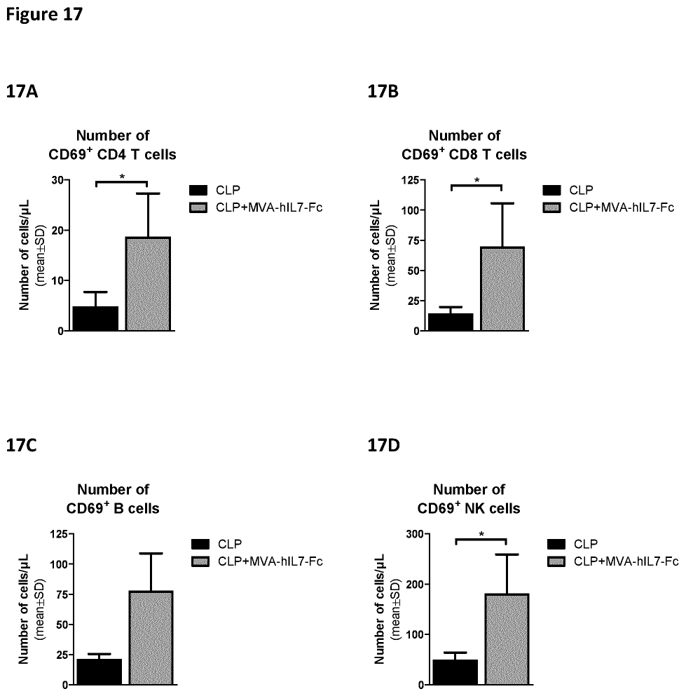

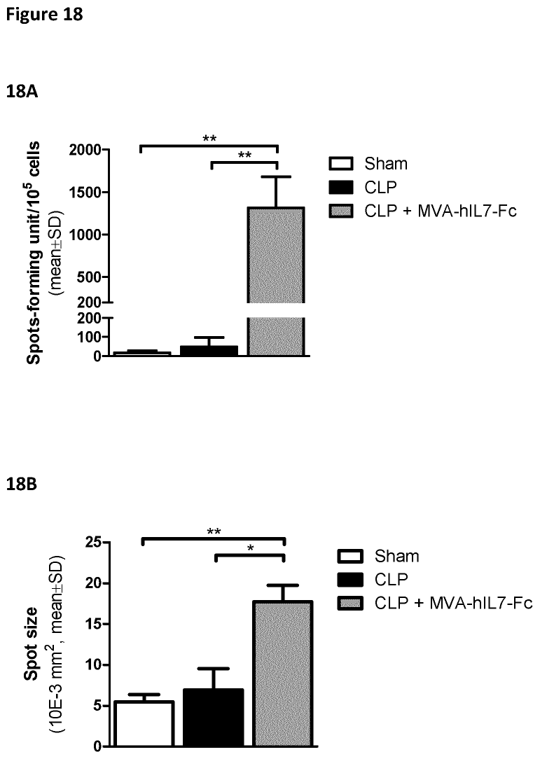

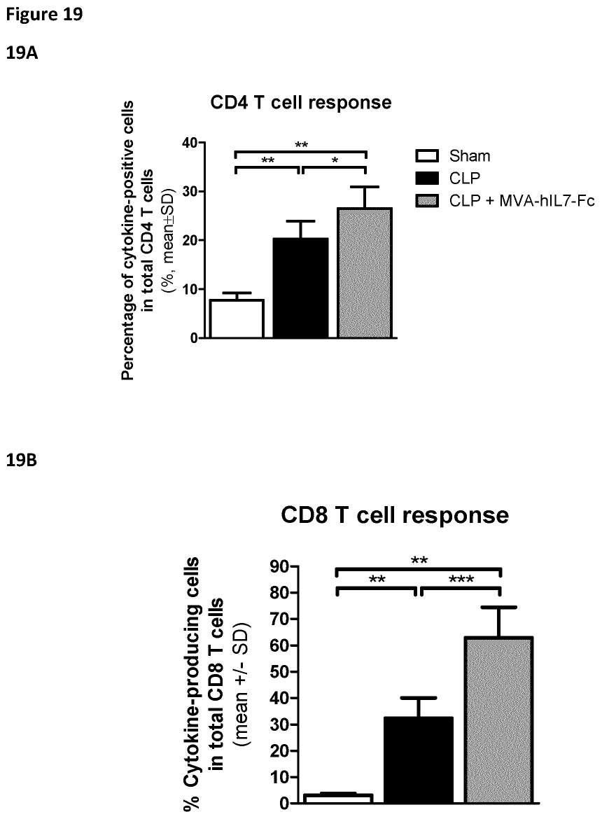

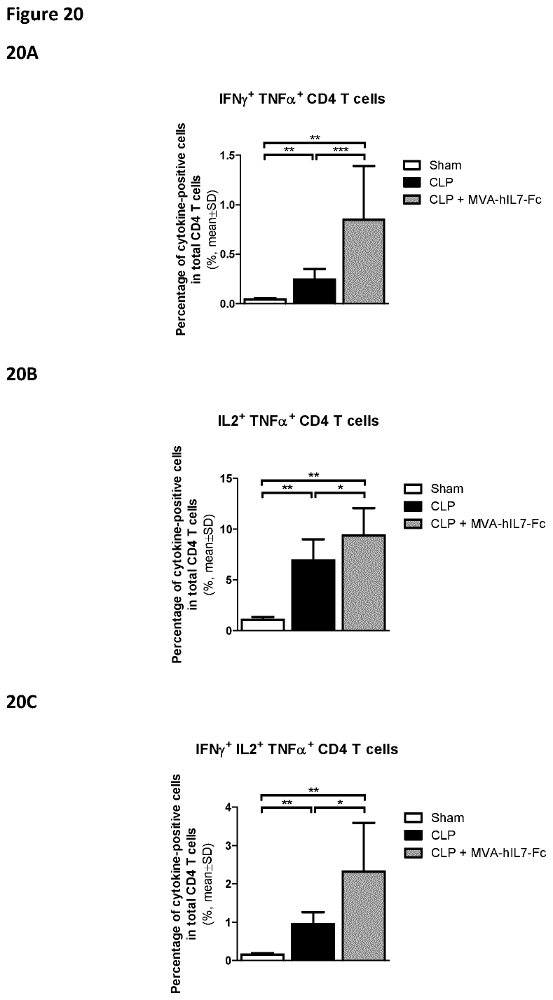

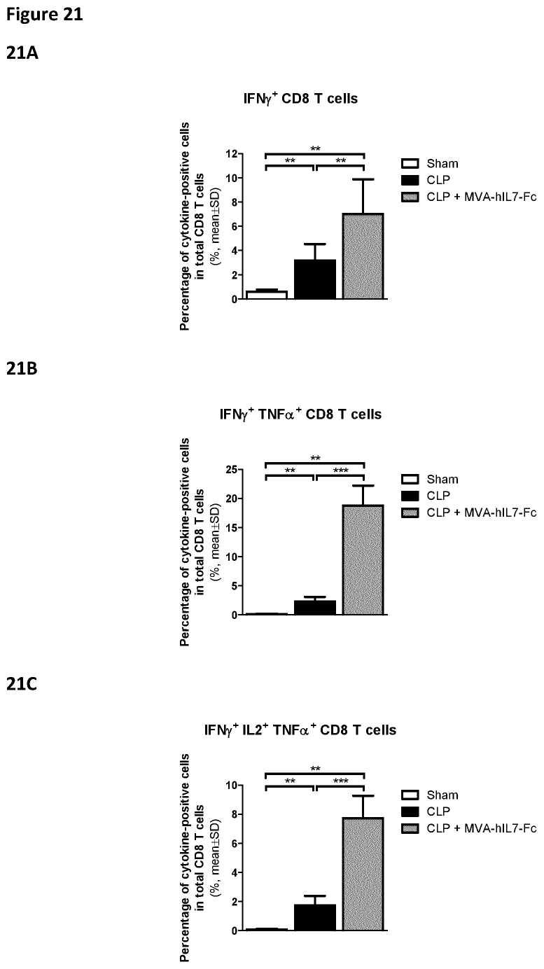

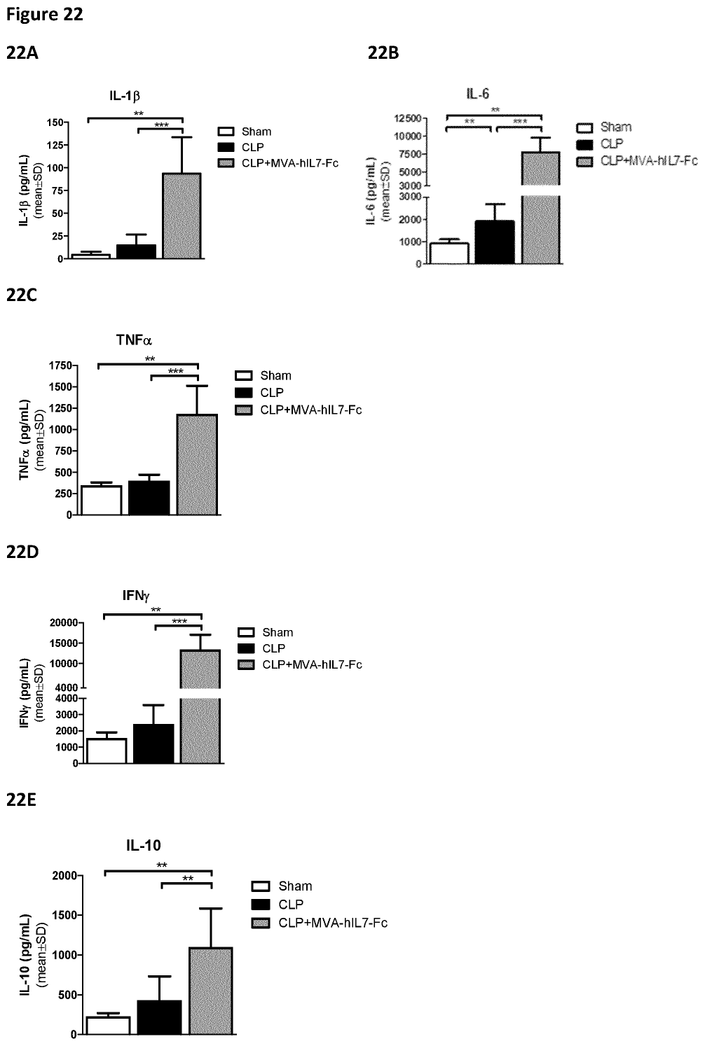

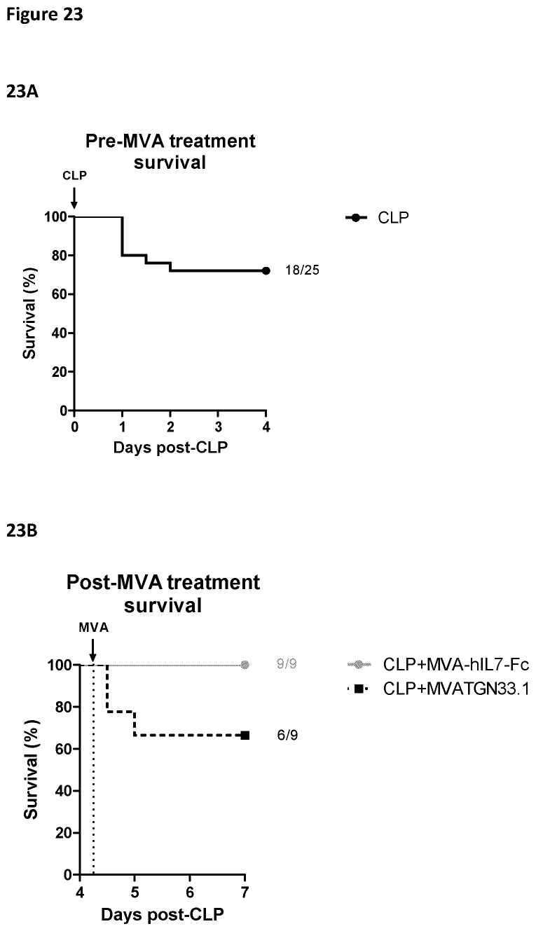

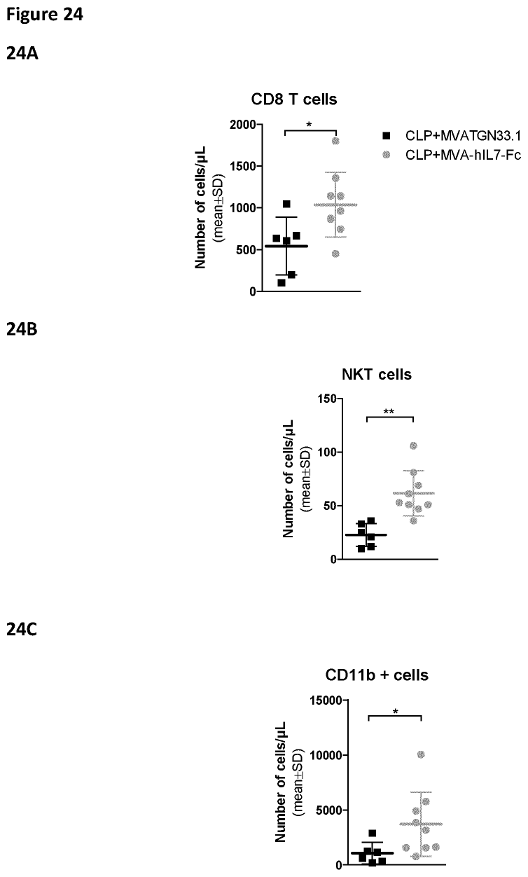

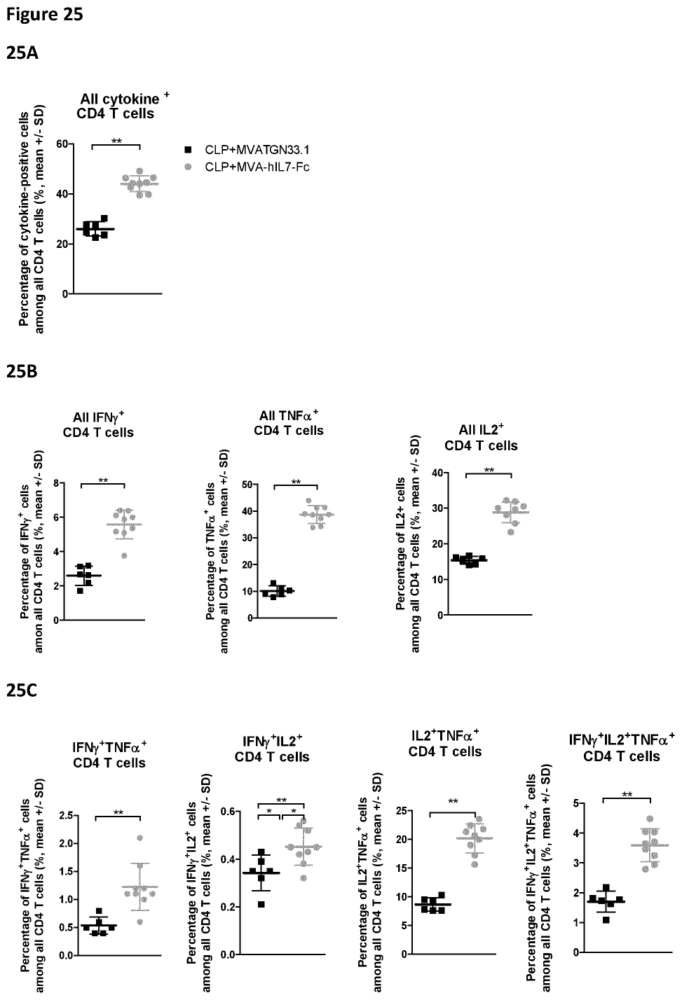

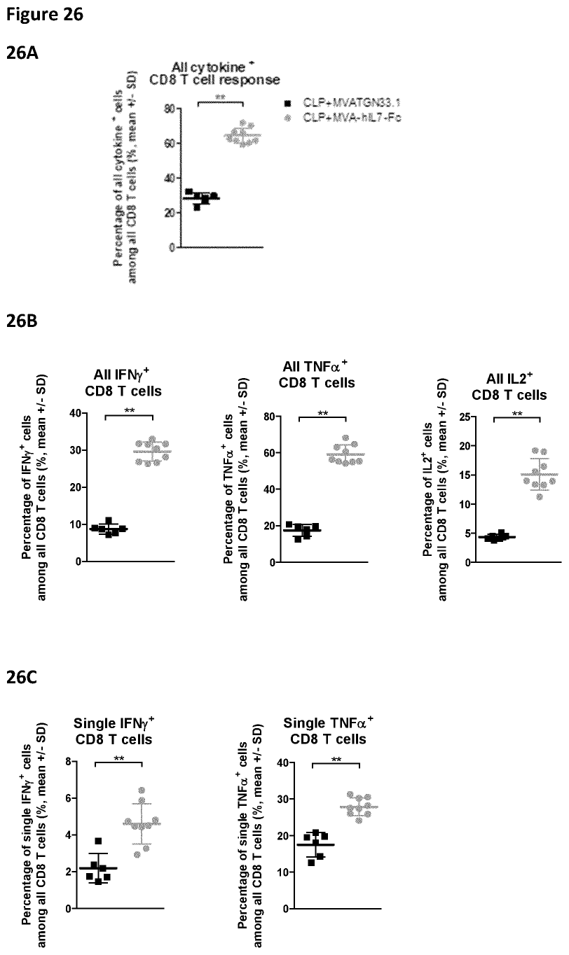

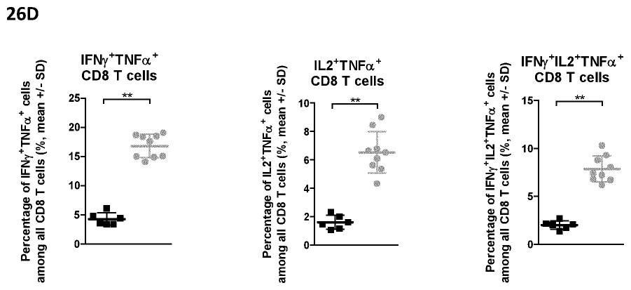

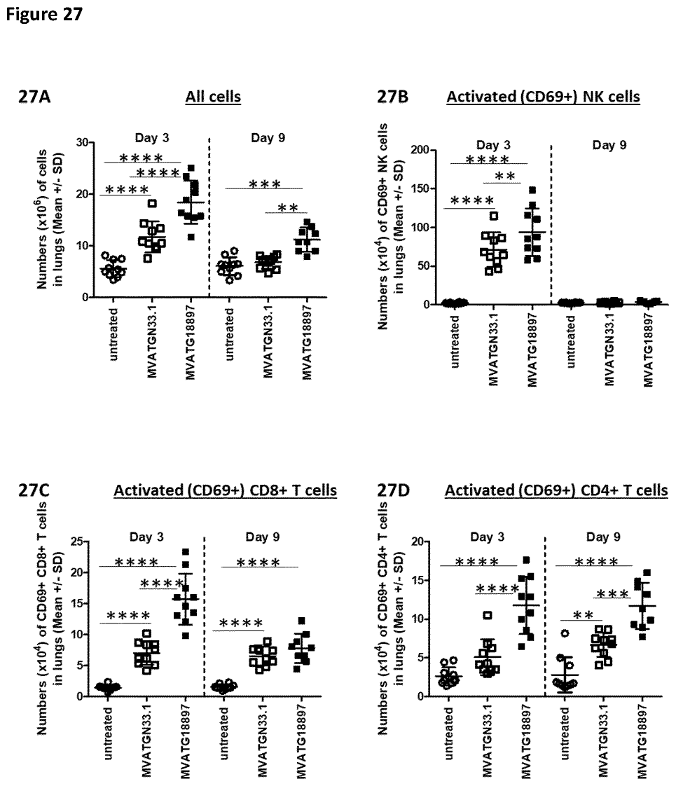

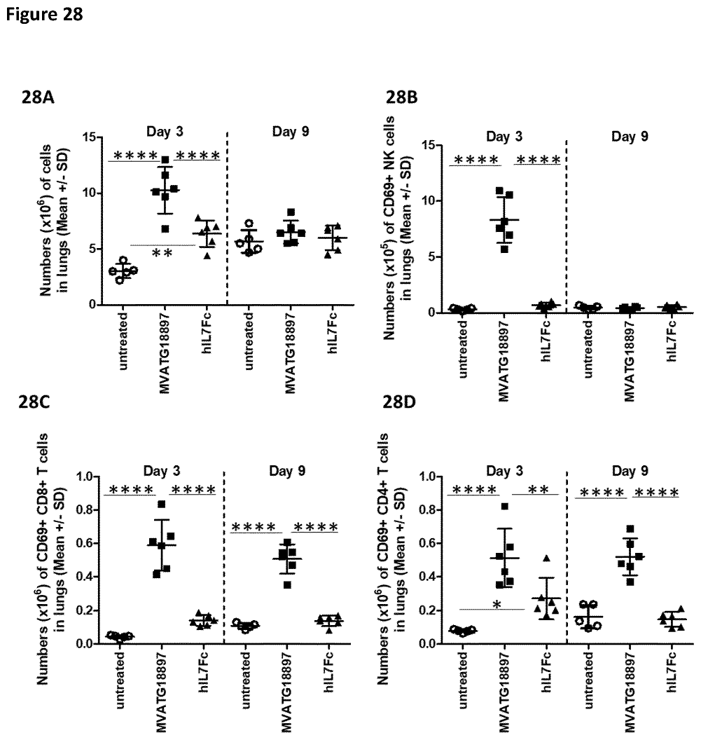

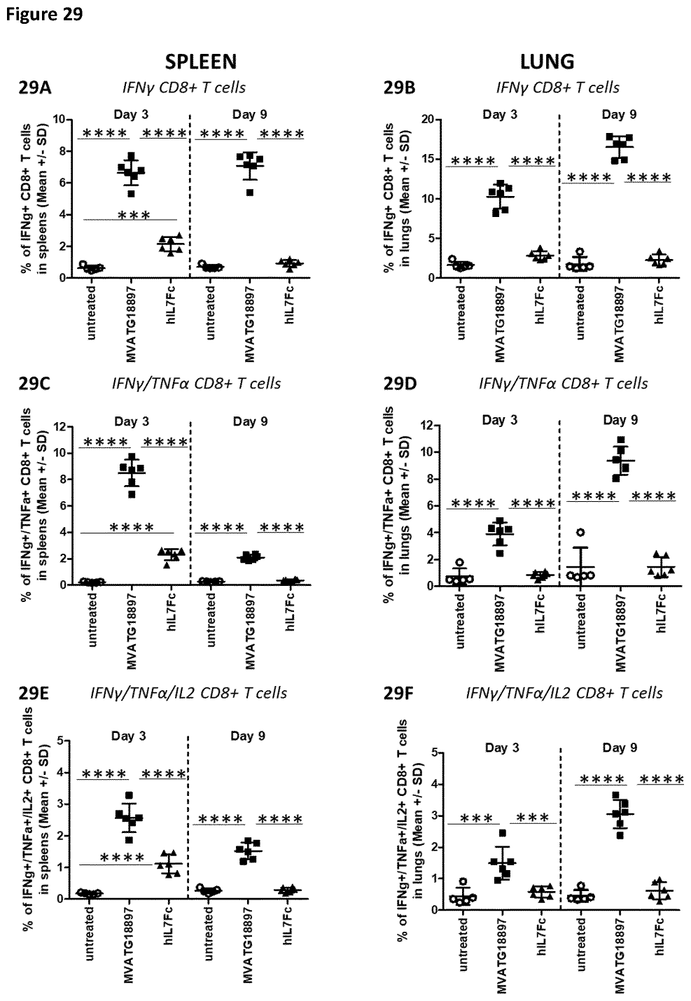

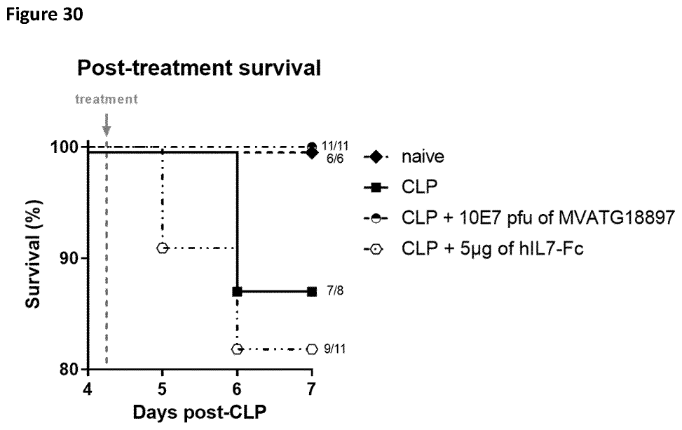

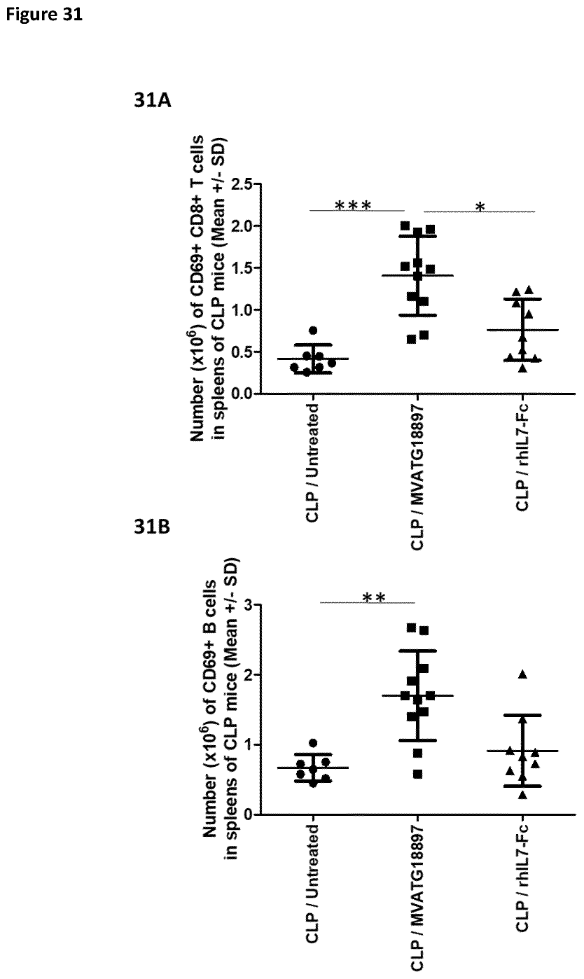

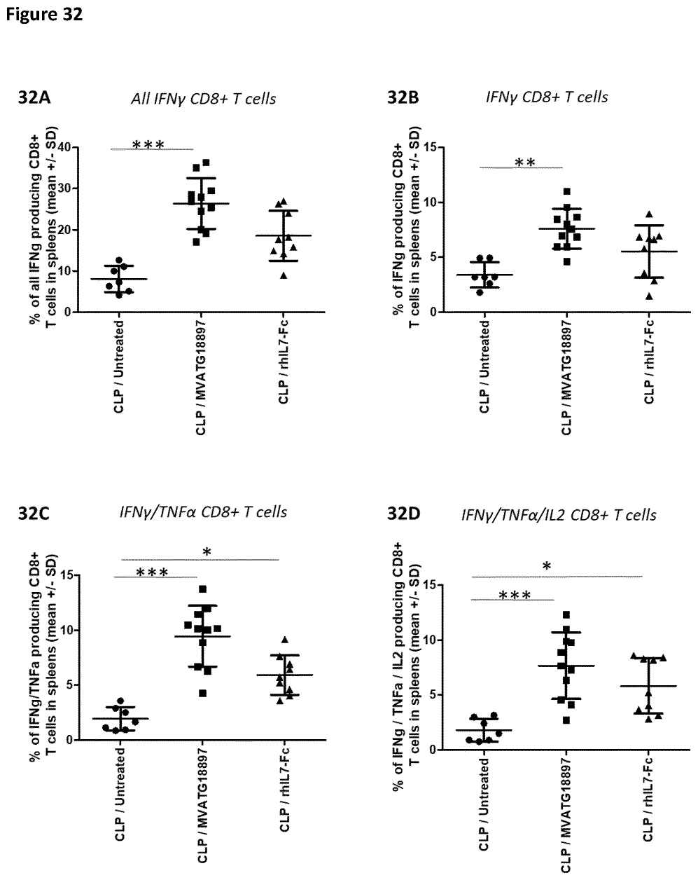

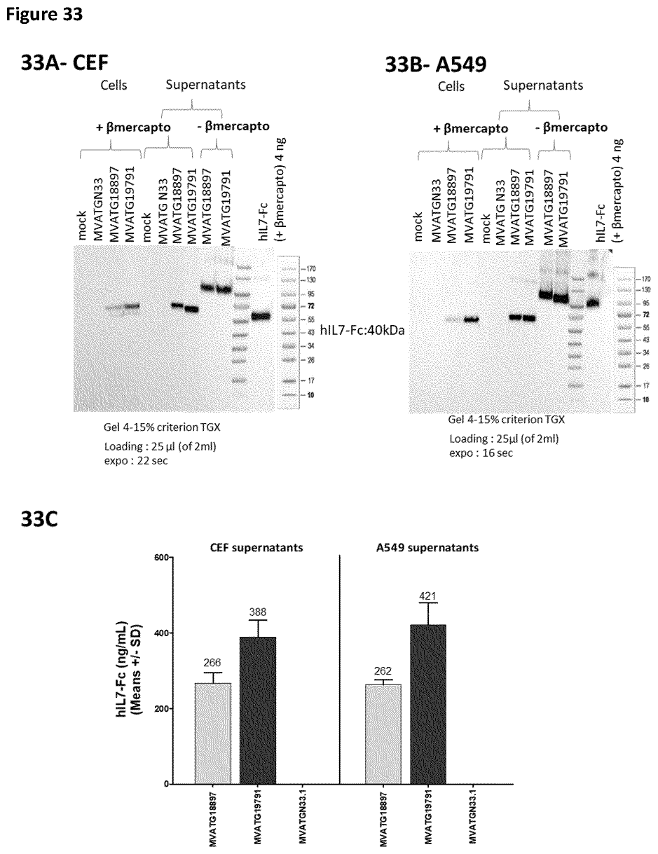

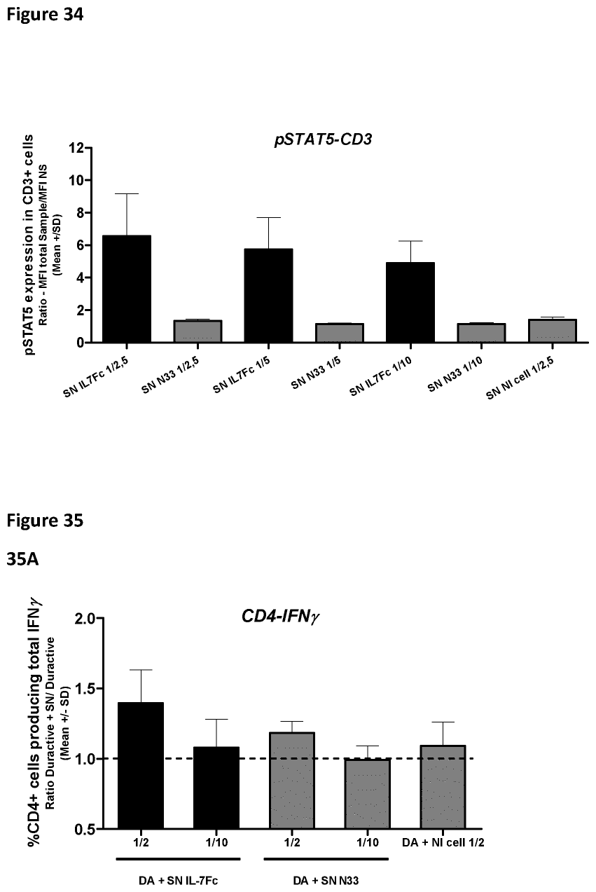

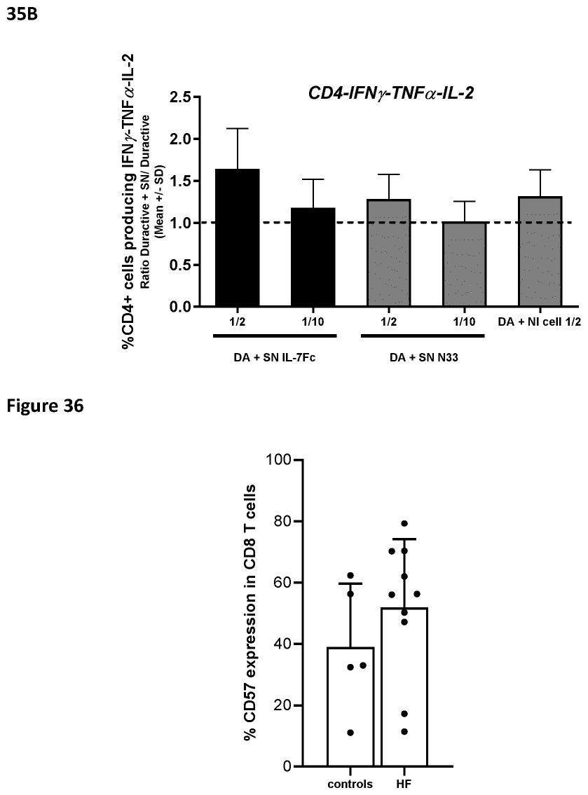

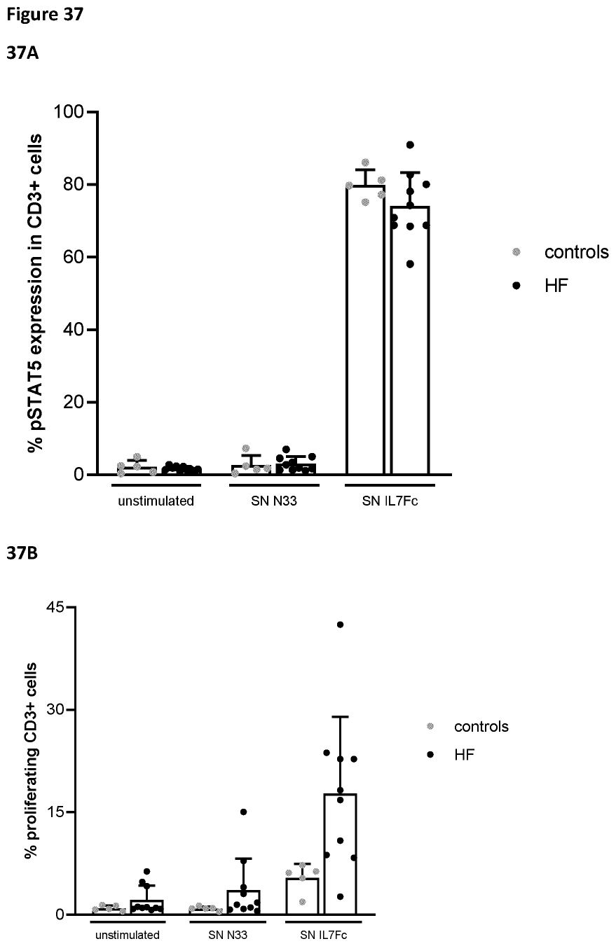

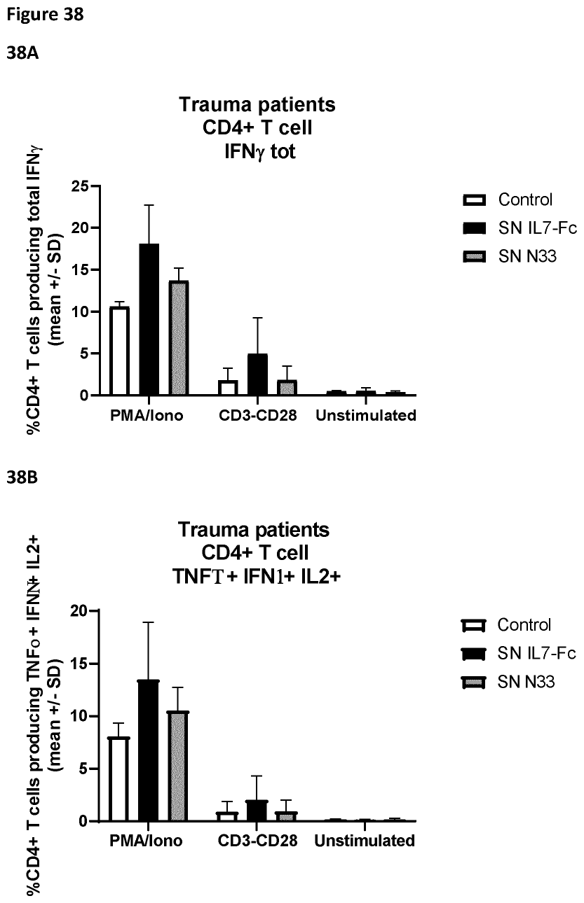

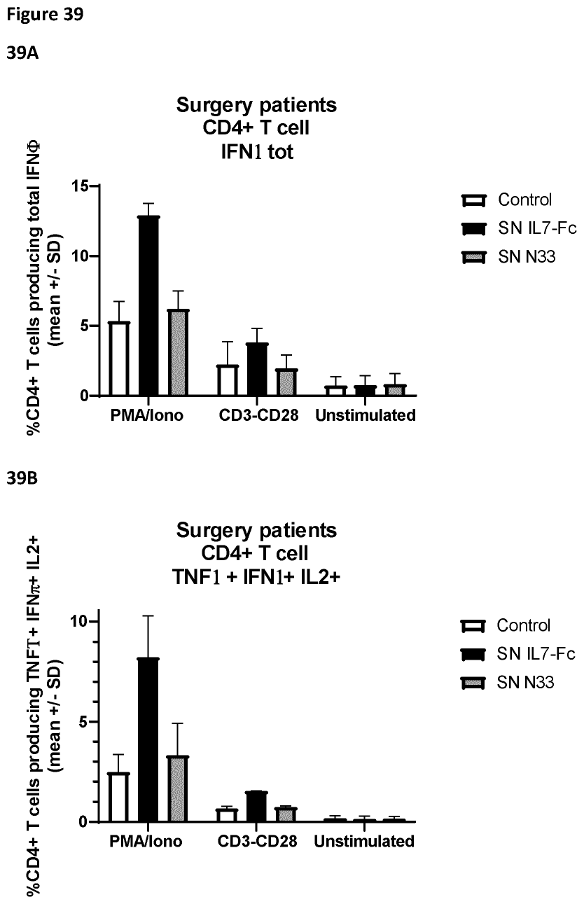

In a first experiment, A549 cells were transduced by empty MVA (MVATGN33.1, negative control) and MVA-hIL-7-Fc (MVATG18897). Cells and supernatants were collected and analyzed by Western Blot (FIG. 1A). No specific bands were detected with cells and supernatants from empty MVA (MVATGN33.1) transduction while with cells transduced by MVA-hIL-7-Fc and the corresponding supernatants, a specific signal was clearly detected. In absence of beta-mercaptoethanol, a smear appeared both for cells and supernatant and in presence of beta-mercaptoethanol, a clear single band appeared for cells and supernatant, slightly upper than 43 kDa due to protein glycosylation, as expected.

In a 2^{nd} experiment, MVA-hIL-7-Fc was used to transduce COS7 cells at various MOI and supernatants were collected at 48 or 72 h. Then, an ELISA detecting human IL-7 was performed on collected supernatants to detect secreted human IL-7 following transduction with MVA-hIL-7-Fc (FIG. 1B). HIL-7 was clearly detected following transduction with MVA-hIL-7-Fc whereas no signal was detected in supernatants of COS7 cells after transduction with the empty MVA. The amount of IL-7 detected within collected supernatants was clearly dose/MOI dependent (25 ng/mL for MOI 0.3, 56 ng/mL for MOI 1 and 66 ng/mL for MOI 3).

The functionality of hIL-7 in supernatant was assessed on PB1 cells, which growth was hIL-7 dependent. An MTT assay measuring cell metabolism was performed on PB1 cells 72 h after an incubation of cells with various dilutions of collected supernatants. As shown in FIG. 1C, no activity was detected with supernatants from empty MVA transduction whereas a signal was detected with supernatants from MVA-hIL-7-Fc-transduced cells. Detected activity was, as expected, dependent on MOI and supernatant dilution. It demonstrates the activity/functionality of the human IL-7 produced by the MVA-hIL-7-Fc after cell infection.

2.2 In Vivo Evaluation of MVA-hIL-7-Fc (MVATG18897) in a Healthy C57BL6/J Mice Model

2.2.1 Experiment 1: Pharmacokinetics of hIL-7 Following Intravenous Injection of MVA-hIL-7-Fc (MVATG18897) at 2 Doses (1×10⁷ and 1×10⁸ pfu)

In this experiment, C57BL6/J mice were divided in 3 groups of 15 animals. The 3 groups were injected respectively by empty MVA (MVATGN33.1) at a dose of 1×10⁸ pfu, and by MVA-hIL-7-Fc (MVATG18897) at a dose of 1×10⁷ pfu or 1×10⁸ pfu, all by intravenous route. The blood of 3 mice per group was sampled at 0 h, 2 h, 6 h, 24 h, 48 h, 72 h, 96 h, 8, 15 and 21 days (sampled mice at each timepoint were different ones due to ethical considerations). Sera were thus used to determine the hIL-7 pharmacokinetics in blood of injected mice using an ELISA as well as circulating mIFNγ using another ELISA.

FIG. 2 shows the detection of circulating hIL-7 and mIFNγ in blood of mice of the different groups. FIG. 2A shows the induction of a slight amount of IFNγ following IV injection with empty MVA due to the induction of innate immunity by the viral vector itself (maximum of 10.7 ng/mL at 6 h post-injection) whereas no hIL-7 was detected as expected. FIG. 2B shows the induction of the same slight amount of IFNγ following IV injection of MVA-hIL-7-Fc at a dose of 10⁷ pfu, due to the viral backbone (maximum of 5.1 ng/mL at 6 h post-injection). In parallel, a peak of circulating hIL-7 was detected as soon as 2 h post injection with a maximum value at 24 h post-injection (2.4 ng/mL). Amount of detected hIL-7 decreased slowly overtime: it was still detectable at 4 days post-injection (0.1 ng/mL) and was no more detected at day 8 post-injection. FIG. 2C shows a similar pattern for mice injected by 10⁸ pfu of MVA-hIL-7-Fc, except that the maximum peak was higher and slightly delayed at 24 h post-injection (24.5 ng/mL at 6 h p.i. and 61.4 ng/mL at 24 h p.i.). Overall amounts of detected hIL-7 were about 1-log higher than with the lower dose. As for the low dose, hIL-7 was still detected at 4 days post-injection (3.1 ng/mL) and no more detected at 8 days post-injection. Pharmacokinetics of circulating mIFNγ was identical to the one observed with the same dose of empty MVA.

This experiment clearly demonstrates the ability of MVA-IL-7 to express detectable levels of circulating hIL-7 over at least 4 days, in comparison with an empty MVA.

2.2.2 Experiment 2: Pharmacokinetics of hIL-7 Following Intravenous Injection of MVA-hIL-7-Fc (MVATG18897) or Empty MVA (MVATGN33.1) and Associated Immunological Activities

In this experiment, C57BL6/J mice were divided in 2 groups of 15 animals. The 2 groups were injected respectively by empty MVA (MVATGN33.1) at a dose of 2×10⁸ pfu, and by MVA-hIL-7-Fc (MVATG18897) at a dose 1×10⁸ pfu, all by intravenous route. The blood of 3 mice per group was sampled at 0 h, 6 h, 24 h, 48 h, 72 h, 96 h, 8, 9, 15 and 29 days (sampled mice at each timepoint were different ones due to ethical considerations). Sera were thus used to determine the hIL-7 pharmacokinetics in blood of injected mice using an ELISA as well as dosing circulating mIFNγ by another ELISA. In addition, 3 mice per group were sacrificed at D1, D3, D9 and D29 and their spleens and thymus were sampled in order to monitor immune cells, their numbers, activation status and phenotypes.

2.2.2.1 hIL-7 and mIFNγ Pharmacokinetics

FIG. 3A shows that no circulating hIL-7 can be detected in mice injected IV by the empty MVA all along the experiment whereas slight amounts of circulating mIFNγ were detected in the first 4 days following injection with a peak at 8.5 ng/ml at 6 hours post injection, reflecting the innate immune response induced by the MVA vector itself.

FIG. 3B shows that mice treated with the MVATG18897 (MVA-hIL-7-Fc) displayed circulating hIL-7 as soon as 6 hours post injection (mean of 23 ng/mL at 6 and 24 hours post injection), slightly decreasing over time (mean of 10, 7, and 5.1 ng/mL at days 2, 3 and 4 post-injection). At day 8 post-injection, circulating hIL-7 was no more detected. The profile of circulating mIFNγ was completely comparable to one observed in mice injected with empty MVA, and thus may be due to the MVA vector itself.

2.2.2.2 Immunological Activities of MVA-hIL-7-Fc (MVATG18897)

2.2.2.2.1 Total Spleen Cells Numbers

FIG. 4 shows the total number of splenocytes depending on groups and time. Both empty MVA (MVATGN33.1) and MVA-hIL-7-Fc (MVATG18897) induced a slight decrease of total spleen cell number at day 1 compared to untreated mice (effect thus probably mediated by the vector itself). At day 3, untreated mice and MVA-hIL-7-Fc-treated mice displayed similar numbers of splenocytes whereas mice treated by empty MVA still displayed a lower number of splenocytes. At day 9, MVA-hIL-7-Fc strongly and significantly increased the number of splenocytes at day 9 (mean of 231×10⁶ cells/spleen) compared to empty MVA (mean of 92×10⁶ cells/spleen) and untreated mice (59×10⁶ cells/spleen). At day 29, the 3 groups displayed comparable number of total splenocytes.

These results presented on FIG. 4 clearly show both an effect of the vector MVA which was able to increase the number of total splenocytes and an effect of the MVA armed with IL-7 which was able to increase strikingly at day 9 the number of splenocytes. MVA-hIL-7-Fc recapitulated the immunological effect of both the vector itself and the arming. The ability of the MVA-hIL-7-Fc to increase the number of spleen cells is expected to be beneficial in immunosuppressed patients by preparing patient's immune system to respond better and more rapidly to a potential new infection or aggression.

2.2.2.2.2 Total Number of CD4 T Cells in Spleen and Sub-Populations of CD4+ T Cells

FIG. 5 shows the total number of CD4 T cells (FIG. 5A) within the spleen depending on groups and timepoints as wells as each subpopulation of CD4+ T cells, id est naïve CD4 T cells (FIG. 5B), CD4+ effector memory cells (FIG. 5C), CD4+ central memory cells (FIG. 5D) and CD4+ acute effector memory cells (FIG. 5E). These 4 sub-populations of CD4+ T cells were identified through the following markers: CD3+CD4+CD62L+CD44− for naïve ones, CD3+CD4+CD62L− CD44+ for effector memory ones, CD3+CD4+CD62L+CD44+ for central memory ones and CD3+CD4+CD62L− CD44− for acute effector ones.

Regarding total CD4+ T cell numbers in the spleens, all 3 groups (untreated, empty MVA-treated and MVA-hIL-7-Fc-treated mice) displayed similar numbers at days 1, 3 and 29. On the contrary, mice treated with empty MVA (MVATGN33.1) displayed a slight increased number of total CD4+ T cells at day 9 compared to untreated mice (20.8×10⁶ versus 13.2×10⁶). Remarkably, mice treated with MVA-hIL-7-Fc (MVATG18897) displayed at day 9 a significantly increased number of total CD4+ T cells in the spleen (32.1×10⁶) compared to untreated mice and compared to mice treated by empty MVA. A higher number of total CD4+ T cells induced by the MVA-hIL-7-Fc is an advantage to treat immunosuppressed patients as it means that more cells from the adaptative arm of immunity are present in the secondary lymphoid organs, ready to respond to a new aggression.

Naïve CD4+ T cells in spleen (FIG. 5B) appeared to be slightly decreased by both empty MVA and MVA-hIL-7-Fc at days 1 and 3 compared to untreated mice (effect related to the MVA vector). On the contrary, the number of naïve CD4+ T cell was significantly increased by MVA-hIL-7-Fc treatment at D9 compared to untreated mice (16.9×10⁶ versus 9.9×10⁶ respectively). At day 29 post-injection, all groups displayed similar number of naïve CD4+ T cells.

Whereas both empty MVA and MVA-hIL-7-Fc decreased number of effector memory CD4+ T cells (FIG. 5C) at day 1 post-injection, only MVA-hIL-7-Fc significantly increased this sub-population at day 3 (3.8×10⁶ cells), compared to empty MVA-treated mice (1.6×10⁶ cells) and at day 9 (10.6×10⁶ cells) (compared to both untreated (1.8×10⁶ cells) and empty MVA-treated mice (5.2×10⁶ cells)). A similar trend was still observed at D29. Of note, empty MVA-treated mice displayed an intermediate increased number of CD4+ effector memory cells at day 9 which was significantly higher than in untreated mice but also significantly lower than in mice treated by MVA-hIL-7-Fc. For the other effector population (acute effector, FIG. 5E), MVA-hIL-7-Fc significantly increased this population at day 9 post-injection (3.8×10⁶ cells) compared to empty MVA (2×10⁶ cells) and to untreated mice (1.4×10⁶ cells).

MVA-hIL-7-Fc also significantly increased the number of CD4+ central memory cells (FIG. 5D) compared to empty MVA at day 3 and 9 post-injection (respectively 1×10⁶ and 1.4×10⁶ at days 3 and 9 versus 0.5×10⁶ and 0.8×10⁶ cells respectively) as well as compared to untreated mice at days 9 and 29 post-injection (1.4×10⁶ and 1.2×10⁶ for MVA-hIL-7-Fc at days 9 and 29 versus 0.4×10⁶ and 0.6×10⁶ cells for untreated mice respectively). No significant effect of the empty MVA compared to untreated mice was observed for this cell sub-population.

MVA-hIL-7-Fc specifically induced, compared to empty MVA, CD4+ effector memory, acute effector and central memory cells in spleens of injected mice at one or several time points post-injection. This demonstrates the effect of the encoded IL-7 in the MVA-hIL-7-Fc. The induction of such subpopulations of CD4 T cells is important in the treatment of immunosuppression, one particular example being treatment of immuno-suppression induced by sepsis, as in particular effector memory and acute effector cells are the cells displaying the fastest specific immune response after a second infection.

2.2.2.2.3 Total Number of CD8+ T Cells in Spleen and Sub-Population of CD8+ T Cells

FIG. 6 shows the total number of CD8 T cells (FIG. 6A) within the spleen depending on groups and timepoints as wells as each subpopulation of CD8+ T cells, id est naïve CD8 T cells (FIG. 6B), CD8+ effector memory cells (FIG. 6C), CD8+ central memory cells (FIG. 6D) and CD8+ acute effector memory cells (FIG. 6E). These 4 sub-populations of CD8+ T cells were identified through the following markers: CD3+CD8+CD62L+CD44− for naïve ones, CD3+CD8+CD62L− CD44+ for effector memory ones, CD3+CD8+CD62L+CD44+ for central memory ones and CD3+CD8+CD62L− CD44− for acute effector ones.

Both empty MVA (MVATGN33.1) and MVA-hIL-7-Fc (MVATG18897) decreased the total number of CD8+ T cells in the spleen at day 1 post-injection compared to untreated mice. Mice treated by empty MVA or MVA-hIL-7-Fc displayed comparable levels, suggesting an effect of the viral vector itself (FIG. 6A). At days 3 and 9, only the MVA-hIL-7-Fc significantly increased the total number of CD8 T cells (13.2×10⁶ and 19.4×10⁶ cells at day 3 and 9 respectively), compared to empty MVA (6.8×10⁶ and 10.2×10⁶ cells at days 3 and 9 respectively) and untreated mice (9.2×10⁶ and 7.7×10⁶ cells for days 3 and 9 respectively), demonstrating a specific effect of the IL-7 encoded in the MVA-hIL-7-Fc on the global CD8+ T cell population of the spleen.

Empty MVA and MVA-hIL-7-Fc also strongly decreased at day 1 post-injection the number of naïve CD8+ T cells (FIG. 6B) and CD8+ T central memory cells (FIG. 6D). At day 3 post-injection, this decrease was still detected for both MVA on naïve CD8+ T cells compared to untreated mice. On the contrary, at the same timepoint (day 3) only MVA-hIL-7-Fc strongly and significantly increased the number of CD8+ effector memory (36.6×10⁶ cell, FIG. 6C), central memory (21.1×10⁶ cells, FIG. 6D) and acute effector (35.6×10⁶ cells, FIG. 6E) cells, compared to empty MVA (respectively 10.9×10⁶, 6.6×10⁶ and 16.5×10⁶ cells) and untreated mice (respectively 4.6×10⁶, 9.2×10⁶ and 16×10⁶ cells). This effect was still detected and significant at day 9 post-injection: for effector memory CD8+ T cells, 33.9×10⁶ versus 11.2×10⁶ versus 2.7×10⁶ cells for MVA-hIL-7-Fc, empty MVA and untreated mice respectively; for memory central CD8+ T cells 35×10⁶ versus 8.4×10⁶ versus 4.3×10⁶ cells for MVA-hIL-7-Fc, empty MVA and untreated mice respectively; for acute effector CD8+ T cells 40.5×10⁶ versus 19.7×10⁶ versus 21.1×10⁶ cells for MVA-hIL-7-Fc, empty MVA and untreated mice respectively. At this timepoint, MVA-hIL-7-Fc also significantly improved the number of naive CD8+ T cells (88.9×10⁶ cells) compared to untreated mice (48.5×10⁶ cells). The effect of MVA-hIL-7-Fc was still significant at day 29 post-injection for CD8+ central memory cells (16.4×10⁶ cells) compared to empty MVA (8.5×10⁶ cells) and untreated mice (7.1×10⁶ cells) (FIG. 6D). Of note, despite the number of CD8+ effector memory cells was slightly increased by the empty MVA at days 3, 9 and 29 post-injection compared to untreated mice, it remained significantly lower than the numbers of such cells induced by MVA-hIL-7-Fc.

MVA-hIL-7-Fc had a major effect on the CD8+ T cells of the spleen and its effect was mainly mediated by the arming, human IL-7. As for CD4+ T cells, the induction of such subpopulations of CD8 T cells is important in the treatment of immunosuppression, one particular example being treatment of immunosuppression post-sepsis, as in particular effector memory and acute effector cells are the cells displaying the fastest specific immune response after a second infection. In addition, the induction of CD8+ central memory T cells up to day 29 after injection is also of interest as these cells are the reservoir of adaptive memory response, for T cell proliferation and conversion in effector T cells.

2.2.2.2.4 Bcl2 Expression in T Cells in Spleen and Thymus

Bcl2 is a gene of survival, the corresponding protein playing a role in the anti-apoptotic process. Bcl2 expression was monitored on T cells within the spleen of mice of this experiment and on thymic cells as well (FIG. 7). The Mean Fluorescence Intensity (MFI) was analyzed by flow cytometry at days 1 and 3 post-injection and on mice from each of the 3 groups.

FIG. 7 shows the expression of Bcl2 on CD4+ T cells (FIG. 7A), CD8+ T cells (FIG. 7B) in spleen and on thymocytes (FIG. 7C) expressed as the MFI. MVA-hIL-7-Fc (MVATG18897) specifically induced a higher Bcl2 MFI at day 1 post-injection on CD4+ T cells in the spleen (442, FIG. 7A), compared to empty MVA (344) and untreated mice (342), which displayed similar levels. At day 1, the MFI of Bcl2 expressed in CD8+ T cells from the spleen (FIG. 7B) was also significantly higher in mice treated by empty MVA (630) compared to untreated ones (483) and significantly higher in mice treated by MVA-hIL-7-Fc (757) than in mice treated with empty MVA and untreated mice. At day 3, the 3 groups looked comparable. In the spleen empty MVA and/or MVA-hIL-7-Fc appeared to rapidly increase the expression of Bcl2 on CD4 and CD8+ T cells.

In the thymus, empty MVA and MVA-hIL-7-Fc increased the expression of Bcl2 at day 3 post-injection (respectively 227 and 246) compared to untreated mice (131). This observed effect was equivalent for empty MVA and MVA-hIL-7-Fc suggesting an effect mainly mediated by the MVA itself.

Overall, empty MVA and/or MVA-hIL-7-Fc improved the expression of the anti-apoptotic protein Bcl2, very early at day 1 post injection in spleen on CD4 and CD8 T cells and at day 3 in the thymus. As it is described in sepsis that immune cells undergo massive apoptosis, it is an interesting potential therapeutic effect of the MVA-hIL-7-Fc to improve the expression of an anti-apoptotic protein such as Bcl2.

2.2.2.2.5 Proportion of Cell Sub-Population within the Thymus

FIG. 8 represents the percentages of each cell subpopulations within the thymus for the 3 groups and depending on the time points (days 1 and 3). Thymic cells were divided in 4 sub-populations: double negative cells (DN, which are CD4− and CD8−), double positive cells (DP, which are CD4+ and CD8+), single positive CD4+ (SP CD4+ which are CD8− and CD4+) and single positive CD8+ (SP CD8+ which are CD8+ and CD4−). The differentiation pathway in the thymus is first DN cells which differentiate into DP cells which differentiate into either SP CD4+ or SP CD8+ and then mature SP CD4+ and SP CD8+ can migrate out the thymus to play their role in the organism. Following treatment with empty MVA (MVATGN33.1) and MVA-hIL-7-Fc (MVATG18897), the proportion of the 4 cell subpopulations was not modified at day 1 post injection compared to untreated mice (FIG. 8A). On the contrary, at day 3 post-injection, empty MVA and MVA-hIL-7-Fc, to a similar extent, decreased the percentages of DP cells (from 73% in untreated mice to 58% and 56% respectively for empty MVA and MVA-hIL-7-Fc) whereas both MVA increased the proportion of SP CD4+ cells (22% for both MVA compared to 12% for untreated mice) and SP CD8+ cells (12% and 13% for empty MVA and MVA-hIL-7-Fc respectively compared to 7% for untreated mice) (FIG. 8B). All percentages came back to values equivalent to untreated mice at days 9 and 29 (not shown). These data suggested that the MVA (empty or encoding IL-7) was able to push the differentiation of T cells from the thymus, as it seemed to increase the percentage of single positive cells (CD4+ or CD8+) which would favor the presence of mature T cells outside the thymus. In a context of immunosuppression post-sepsis, a lymphopenia is observed and one of the aims of therapy in this immunosuppressive phase is to restore a normal lymphocytes count. The effect of the MVA-hIL-7-Fc on the thymus and on differentiation of T cells is expected to be beneficial in the restoration of normal lymphocytes counts.

2.2.2.2.6 Numbers of Neutrophils and Myeloid Dendritic Cells

FIG. 9 represents the numbers of neutrophils (FIG. 9A) and of myeloid dendritic cells (mDC) (FIG. 9B) in spleen depending on treatment and timepoints. At day 1, both empty MVA (MVATGN33.1) and MVA-hIL-7-Fc (MVATG18897) tended to increase the number of neutrophils compared to untreated mice. Remarkably, MVA-hIL-7-Fc induced a significant increase in numbers of neutrophils at days 3 (1.9×10⁶ cells) and 9 (1.1×10⁶ cells) post-injection compared to untreated mice (0.49×10⁶ cells at day 3 and 0.3×10⁶ cells at day 9) and/or to empty MVA (0.42×10⁶ cells at day 3 and 0.6×10⁶ cells at day 9). All 3 groups displayed comparable number of neutrophils at day 29. Neutrophils are important cells playing a key role in host's defense against various attacks including infection. They can be altered in immunosuppression situations, like for example in immunosuppression induced by sepsis. Hence, capacity of MVA-hIL-7-Fc to induce neutrophils represents a strong asset to reach restoration of homeostasis of neutrophils.

In parallel, monitoring of mDC in spleen showed a significant decrease of mDC by both empty MVA (1.3×10⁵ cells) and MVA-hIL-7-Fc (3.2×10⁵ cells) at day 3 post-injection compared to untreated mice (9.3×10⁵ cells). One hypothesis for this decrease is the possible infection of mDC by MVA which led to the apoptosis of such cells (induced at 48 h post-infection by MVA) while they expressed within the spleen the encoded hIL-7. On the contrary at day 9, MVA-hIL-7-Fc specifically induced a significant increase in mDC (14.4×10⁵ cells) compared to empty MVA (6.1×10⁵ cells) and untreated mice (4.6×10⁵ cells), while numbers of mDC in untreated mice and mice treated by empty MVA were comparable (decrease induced by empty MVA was restored). Numbers of mDC were comparable at day 29 among groups, observed effects were transient. During immunosuppression, and in particular immunosuppressive phase induced by sepsis, mDC numbers are decreased. The effect of MVA-hIL-7-Fc on these cell numbers at day 9 is attractive to restore the numbers of such cells.

2.2.2.2.7 Numbers of Subpopulations of Monocytes

During immune depression, and in particular immune depression induced by sepsis, monocytes are clearly affected. Despite the fact that IL-7 is more described to be active on T cells, effect of the MVA-hIL-7-Fc (MVATG18897) and its control empty MVA (MVATGN33.1) on monocytes was monitored. In particular the 3 sub-populations of monocytes already described in mice were stained: the ones being pro-inflammatory and exerting phagocytosis (displaying Ly6C^{high}) (FIG. 10A), the ones being only pro-inflammatory (Ly6C^{int}) (FIG. 10B) and the ones described as patrollers in tissues/macrophages (displaying Ly6C^{low}) (FIG. 10C). The natural way of differentiation for a monocyte is from the Ly6C^{high} stage to the Ly6C^{low} stage.

In this experiment, as for other cell populations described in above paragraphs, empty MVA and MVA-hIL-7-Fc induced at day 1 a slight decrease in the number of the 3 sub-populations of monocytes in spleen compared to what was observed in untreated mice, effect probably mediated by the MVA vector. For Ly6C^{high} monocytes, MVA-hIL-7-Fc specifically induced a strong and significant increase of these monocytes at day 3 (185.5×10⁴ cells), compared to empty MVA (63.9×10⁴ cells) and untreated mice (36.6×10⁴ cells). Numbers of these Ly6C^{high} monocytes became again comparable among the 3 groups at days 9 and 29. For Ly6C^{int} monocytes, MVA-hIL-7-Fc also induced a strong and significant increase at day 3 (106.4×10⁴ cells) and day 9 (192.5×10⁴ cells) compared to empty MVA and untreated mice (respectively 42.3×10⁴ and 23.3×10⁴ at day 3 and 53.2×10⁴ and 8.8×10⁴ cells at day 9). Of note at day 9, empty MVA also induced an increase of these cells compared to untreated mice. Numbers of these monocytes became again comparable among groups at day 29. For the Ly6C^{low} monocytes, MVA-hIL-7-Fc also specifically induced a significant increase in these monocytes at day 3 (61.1×10⁴ cells) and day 9 (68.54×10⁴ cells) compared to empty MVA and untreated mice (respectively 28.5×10⁴ and 29.1×10⁴ cells at day 3 and 17.4×10⁴ and 11.7×10⁴ cells at day 9). Similarly to other monocyte subpopulation, these sub-population numbers became comparable among groups at day 29.

These data demonstrated that MVA-hIL-7-Fc (MVATG18897) had an activity on monocytes, which appeared mainly mediated by the arming IL-7 (except for Ly6C^{int} monocytes which appeared to be increased also by empty MVA but to a lower level than MVA-hIL-7-Fc). It first induced Ly6C^{high} monocytes (“immature monocytes”) at day 3 post-injection which disappeared at the later timepoints. Other monocytes Ly6C^{int} and Ly6C^{low} were increased as soon as day 3 too but displayed an equivalent or even stronger increase at day 9 while Ly6C^{high} monocytes got back to “normal” values observed in untreated mice. This observation can suggest that MVA-hIL-7-Fc induces monocytes that then differentiate between day 3 and 9 in Ly6C^{int} and Ly6C^{low} monocytes. All groups are again comparable at day 29, indicating that this induction was transient. Regarding the altered features of monocytes during sepsis-induced immunosuppression, observed effects of the MVA-hIL-7-Fc on monocytes are of particular interest.

2.2.3 Experiment 3: Activity of MVA-hIL-7-Fc (MVATG18897) and Empty MVA (MVATGN33.1) on Lung Cells of Healthy C57B16/J Mice

2.2.3.1 Number of Lung Cells

After lung preparation, the overall recovered cells were counted using the Muse Cell analyzer (FIG. 27A). At day 3 post-injection, MVATGN33.1 significantly increased the numbers of lung cells compared to untreated mice. MVA-hIL-7-Fc also significantly improved this cell number compared to untreated mice. In addition, the increase in lung cell number in MVA-hIL-7-Fc treated mice was significantly higher than the one observed for MVATGN33.1-treated mice, suggesting a stronger activity of the armed MVA. At day 9 post-injection, mice treated by MVATGN33.1 did not display any increase in cell lung numbers compared to untreated ones whereas MVA-hIL-7-Fc-treated mice still display a significant increase in cell lung numbers, which is significantly higher than the ones of untreated or MVATGN33.1-treated mice.

2.2.3.2 Activated NK, CD8+ and CD4+ T Cell Numbers in Lungs

Numbers of NK, CD8+ and CD4+ T cells that were activated (e.g.: displayed CD69 marker) among lung cells were assessed by flow cytometry.

Both MVATGN33.1 and MVA-hIL-7-Fc significantly enhanced the numbers of activated NK cells at day 3 post-injection when compared to numbers of activated NK cells in untreated mice (FIG. 27B). MVA-hIL-7-Fc even significantly increased these numbers of activated NK when compared to MVATGN33.1, suggesting a significant effect of the arming. At day 9 post injection, all 3 groups were equivalent in numbers of CD69+NK cells.

MVATGN33.1 and MVA-hIL-7-Fc also significantly increased the numbers of activated CD8+ T cells at day 3 post-injection when compared to untreated mice (FIG. 27C). The increase induced by MVA-hIL-7-Fc was significantly stronger than the one induced by MVATGN33.1, suggesting again a significant activity of the hIL-7-Fc arming on this parameter. At day 9 post injection, mice treated with MVATGN33.1 or MVA-hIL-7-Fc still displayed a significantly higher numbers of CD69+CD8+ T cells compared to untreated mice. Nonetheless there was no more statistically significant difference between MVA-hIL-7-Fc and MVATGN33.1-treated mice.

For activated CD4+ T cells, only MVA-hIL-7-Fc displayed a significant increase of their numbers compared to untreated mice and MVATGN33.1-treated mice, suggesting an effect of the arming at this timepoint. At day 9 post-injection, MVATG18897 and MVATGN33.1 were shown to improve the CD69+CD4+ T cell numbers compared to untreated mice and the difference between MVATGN33.1 and MVA-hIL-7-Fc was also significant with a higher number of cells in MVA-hIL-7-Fc treated mice.

2.2.4 Experiment 4: Activity of MVA-hIL-7-Fc (MVATG18897) and hIL-7-Fc Protein on Spleen and Lung Cells of Healthy C57Bl6/J Mice

2.2.4.1 Numbers of Lung Cells

After lung preparation, the overall recovered cells were counted using the Muse Cell analyzer (FIG. 28A). At day 3 post-injection, both hIL-7-Fc and MVATG18897 significantly improved the overall cell numbers in lung. Nonetheless, numbers were much more and significantly higher in mice treated with MVATG18897. At day 9 post-injection, the 3 groups, untreated or treated, displayed similar numbers of lung cells.

2.2.4.2 Numbers of Activated (CD69+) NK, CD8+ and CD4+ T Cells in Lungs

Numbers of activated (CD69+) NK cells were strongly and significantly increased in lungs of mice treated by MVATG18897, compared to untreated mice at day 3 post-injection (FIG. 28B). These numbers came back to levels comparable to those of untreated mice at day 9 post injection. In parallel, hIL-Fc protein did not display any activity on these activated cells, as number of such cells were similar to those of untreated mice at days 3 and 9 post-injection.

Regarding CD69+CD8+ T cell, hIL-7-Fc protein tended to increase their numbers at day 3 (despite this was not significant) (FIG. 28C). These numbers were equivalent to untreated mice at day 9 post injection. In parallel, MVATG18897 strongly and significantly increased their numbers at day 3 post-injection compared to untreated mice and mice treated with hIL-7-Fc. At day 9 post injection, MVATG18897-treated mice still displayed a significantly higher numbers of CD69+CD8+ T cells when compared to untreated mice and hIL-7-Fc protein-treated mice.

Regarding CD69+CD4+ T cells, hIL-7-Fc significantly improved their numbers 3 days post-injection when compared to untreated mice (FIG. 28D). No more effect of the hIL-7-Fc on these cells was observed 9 days post-injection. MVATG18897 strongly and significantly enhanced their numbers at day 3 post injection when compared to untreated mice, and also when compared to hIL-7-Fc treated ones. At day 9 post-injection, MVATG18897 still induced a significant increase of the numbers of CD69+CD4+ T cells when compared to both untreated and hIL-7-Fc-treated mice.

On these 3 cells populations in lungs, MVATG18897 activity is clearly significantly higher than the one of the hIL-7-Fc protein, demonstrating the interest to vectorize the hIL-7-Fc in such a viral vector. This is of particular interest as most of secondary infections in immunosuppressed septic patients are pulmonary ones and these induced activated immune cells will rapidly initiate an immune response, directly on the site of infection.

2.2.4.3 Ability of CD8+ T Cells to Produce Cytokines in Spleens and Lungs.

Functionality of CD8+ T cells in spleens and in lungs was monitored through an ICS after TCR stimulation.

In spleen, at day 3, the percentage of CD8+ T cells producing only IFN-gamma (FIG. 29A) was significantly increased by both hIL-7-Fc protein and MVATG18897 when compared to untreated mice (mean of 0.6%). The increase induced by MVATG18897 (mean value of 6.5%) is significantly and dramatically higher than the one induced by hIL-7-Fc (mean value of 2%). At day 9, mice treated by hIL-7-Fc protein displayed percentages similar to untreated mice (mean values about 0.6% to 0.9%) while MVATG18897-treated mice still displayed significantly higher percentages of IFNγ-producing CD8+ T cells (mean value of 7.5%). A similar observation can be done for CD8+ T cell producing both IFNγ and TNFα (FIG. 29C). hIL-7-Fc slightly but significantly increased their percentages at day 3 post injection (mean value of 2.3%) compared to untreated mice (mean value of 0.2%) and there was no more effect at day 9 post injection. MVATG18897 on its side, strongly and significantly increased the percentages of these cells (mean value of 8.5%), when compared to untreated and hIL-7-Fc treated mice at day 3. This effect, despite being less important is still clearly significant at day 9 post-injection.

For CD8+ T cells producing 3 cytokines (IFNγ and TNFα and IL2) (FIG. 29E), hIL-7-Fc protein significantly increased their percentages at day 3 post injection (mean value of 1.2%) when compared to untreated mice (mean value of 0.5%) and this effect was no more observed at day 9 post injection. MVATG18897 induced a strong and significant increase in the percentages of these polyfunctional cells with mean value of 2.6% and 1.5% at days 3 and 9 post-injection respectively. In spleen, MVATG18897 was significantly superior to hIL-7-Fc protein to induce CD8+ T cells able to produce cytokines after TCR stimulation. These functional T cells induced by MVAtG18897 will be able to contribute significantly to the control of a potential future secondary infection.

In lungs, while active in spleens, hIL-7-Fc was not capable of increasing the percentages of CD8+ T cells producing either only IFNγ, or IFNγ and TNFα or IFNγ, TNFα and IL2, when compared to untreated mice, at day 3 and day 9 post-injection (FIGS. 29B, 29D and 29F). Only a slight trend can be observed for IFNγ-producing CD8+ T cells 3 days post-injection (mean value of 2.8% compared to 1.8% for untreated mice). At the opposite, MVATG18897 was able to strongly and significantly increase percentages of IFNγ, IFNγ/TNFα and IFNγ/TNFα/IL2 producing CD8+ T cells in lungs at days 3 and 9 post injection when compared to untreated mice and hIL-7-Fc treated mice.

In lungs MVATG18897 is capable of boosting CD8+ T cell ability to produce cytokine, while the hIL-7-Fc protein is only very poorly active on the functionality of CD8+ T cells in this organ. The presence of functional CD8+ T cells in lungs is foreseen as a clear advantage in case of pulmonary secondary infection, as these functional cells will be able to control rapidly the infection directly on the potential infection site, through at least production of cytokines.

Overall, the comparison of MVATG18897 and hIL-7-Fc showed that hIL-7-Fc was active on some cells in spleen but MVATG18897 displayed a clearly stronger activity in this organ. Presence of numerous activated and functional immune cells in spleens due to MVATG18897 suggests that in case of secondary infection, the overall immune system will more rapidly be able to fight against a secondary infection. In addition, only MVATG18897 was strongly and significantly active on immune cells in lung, in particular on the functionality of T cells. Presence of such cells, ready to initiate a quick and strong immune response, directly on the site of potential secondary infections is an additional advantageous feature of MVATG18897. These demonstrate the difference and superiority of the MVATG18897 on hIL-7-Fc in healthy mice.

2.3 In Vivo Evaluation of MVATG18897 in CLP Mice

2.3.1 Survival

Survival of mice was followed from DO to D4, i.e. before MVA-hIL-7-Fc (MVATG18897) treatment at D4, in all CLP mice and control Sham-operated mice (FIG. 11A). Twenty-five out of 33 CLP mice (76%) were still alive at D4 as compared to control Sham group in which no death was observed (5/5 mice). At D4, surviving CLP mice were then separated in two groups: CLP mice administered with MVA-hIL-7-Fc at D4 (n=15) and control CLP mice left untreated (n=10). At D7, while 7/10 untreated CLP mice (70%) were still alive, all CLP mice treated with MVA-hIL-7-Fc survived (FIG. 11B).

This result shows that MVA-hIL-7-Fc is active and significantly improves survival of CLP-induced septic mice, suggesting that MVA-hIL-7-Fc may improve the host immune system to fight against the infection.

2.3.2. Circulating hIL-7-Fc

Level of circulating hIL-7-Fc produced in CLP mice treated with MVA-hIL-7-Fc (MVATG18897) was determined by hIL-7-specific ELISA at D7, i.e. 3 days following MVA-hIL-7-Fc treatment. Significant human IL-7 (9600 pg/mL) was measured in sera of MVA-hIL-7-Fc-treated CLP mice (FIG. 12). As expected, undetectable level of hIL-7 was observed in sera of both untreated CLP and Sham mice. This demonstrated that MVA-hIL-7-Fc can transduce cells in immunodepressed CLP mice and that the hIL-7-Fc transgene was well expressed by the MVA and released in blood to detectable levels

2.3.3 Biological Activities of MVATG18897 in CLP Mice

Capacity of MVA-hIL-7-Fc (MVATG18897) to restore or to boost immune system of CLP-induced immunosuppressed septic mice was then assessed. Three days after MVA-hIL-7-Fc administration, overall inflammatory status and immune cells compartments in treated CLP mice were assessed in comparison to untreated CLP mice and control Sham mice.

2.3.3.1. Overall Inflammatory Status

The global inflammatory status of animals was determined by dosing both pro-inflammatory and anti-inflammatory cytokines in sera of septic CLP-operated mice treated with MVA-hIL-7-Fc (MVATG18897).

Seven days after surgery and 3 days post treatment by MVA-hIL-7-Fc, level of IFNγ in sera of Sham, untreated CLP and MVA-hIL-7-Fc-treated CLP mice was assessed (FIG. 13). While level of IFNγ was below lower limit of detection in Sham mice and at a low value in untreated CLP group (0.6 and 1.9 pg/mL, respectively), a significant higher level of IFNγ was measured in sera of CLP mice treated with MVA-hIL-7-Fc (12.4 pg/mL). Since therapeutic effects of IFNγ treatment have been reported in critically ill septic patients, induction of circulating IFNγ by MVA-hIL-7-Fc treatment is of particular interest in improving the immune system of these patients.

These results demonstrate that MVA-hIL-7-Fc does not exacerbate the overall inflammatory status of septic animals showing its innocuity when administered 4 days after sepsis induction, without major change in the cytokines measured in sera, except for IFNγ. Interestingly, MVA-hIL-7-Fc promotes production of IFNγ in blood that may contribute to improve the impaired host immune system.

2.3.3.2. Characterization of Immune Cell Subsets in Spleen

2.3.3.2.1. Cell Phenotyping

Effects of MVA-hIL-7-Fc (MVATG18897) treatment on restoration of impaired cell compartments in spleen of immunosuppressed CLP mice were determined by flow cytometry. FIG. 14 shows composition of different cell subsets in spleen. As observed in the literature, CLP mice presented a splenomegaly seven days after surgery. Total splenocytes were indeed 2.2-fold higher in CLP mice than in Sham mice (144 and 73×10⁶ cells, respectively; FIG. 14A). MVA-hIL-7-Fc treatment led to a similar number of total splenocytes (70×10⁶ cells) as in Sham mice. This result is interesting since it demonstrates that MVA-hIL-7-Fc helps immune system of septic CLP mice recovering a normal physiological condition.

Total T cells (CD3⁺) decreased dramatically in CLP mice compared with Sham mice (7 and 25×10⁶ cells, respectively), which is a hallmark of the immunosuppression induced in septic CLP mice (FIG. 14B). In CLP mice administered with MVA-hIL-7-Fc, T cell compartment was significantly and partially recovered (12×10⁶ cells) showing that MVA-hIL-7-Fc was effective in CLP mice since one of the main known effects of IL-7 is to induce T cell proliferation even in immunodepressed subjects.

Focusing on CD3+ sub-populations, being C8+ and CD4+ T cells, both are strongly decreased in untreated CLP mice compared to Sham ones (FIG. 14C and D). MVA-hIL-7-Fc significantly improves the number of CD8+ T cells compared to CLP untreated mice, partially restoring their numbers compared to Sham mice (FIG. 14D). The effect of MVA-hIL-7-Fc on the CD4+ T cell population is less obvious, despite a trend to increase these cell population (FIG. 14C).

MVA-hIL-7-Fc (MVATG18897) is able to restore a normal cell number within spleens of CLP mice which is linked to sepsis induced-pathogenesis in this model as well as in humans. More specifically, it is also able to partially restore the T cell numbers in spleen (CD3+ T cells as a whole and in particular CD8+ T cells within this population) which were strongly decreased in untreated CLP mice. Overall, the armed-MVA was shown to restore at least partially the immune homeostasis of T cells in spleen within immunosuppressed septic mice.

2.3.3.2.2. Cell Activation

Effects of MVA-hIL-7-Fc (MVATG18897) treatment on activation of immune cells were investigated through quantification of CD69-expressing cells by flow cytometry. FIG. 15 shows the number and the percentage of CD69+ cells in different cell subsets of spleen.

Absolute counts of CD69⁺ B cells (FIG. 15A) increased significantly in spleen of MVA-IL-7-Fc treated CLP mice (4×10⁶ cells) when compared to Sham mice (0.9×10⁶ cells) and CLP untreated mice (1.9×10⁶ cells).

Regarding CD69⁺ CD4 T cells, while their number was similar in both Sham and CLP untreated mice (1.1 and 0.8×10⁶ cells, respectively), CD69+CD4 T cell count improved significantly in MVA-hIL-7-Fc-treated CLP mice as compared with untreated CLP mice (1.3×10⁶ cells; FIG. 15B).

While the numbers of CD69⁺ CD8 T cells were similar between Sham and CLP mice (0.35 and 0.25×10⁶ cells, respectively; FIG. 15C), an important increase of activated CD69⁺ CD8 T cell number was measured in CLP mice treated with MVA-hIL-7-Fc (1.04×10⁶ cells).

The number of NK cells expressing the activation marker CD69 in Sham and CLP untreated mice was similar again (0.2 and 0.3×10⁶ cells, respectively; FIG. 15D), and increased significantly after treatment with MVA-hIL-7-Fc (0.8×10⁶ cells).

Altogether, these data demonstrate that MVA-hIL-7-Fc treatment 4 days following induction of sepsis induces a partial restoration of CD4 and CD8 T cell compartments that was significant for the latter in spleen of septic immunosuppressed CLP mice. This result is important since one of the major concerns in critically ill septic patients is the recovery of T cell compartments. Moreover, administration of MVA-hIL-7-Fc in CLP mice enhanced the number of activated cells in various cell subsets, including T, NK and B cells, that may contribute to fight against abdominal infection.

2.3.3.3. Characterization of Immune Cell Subsets in Blood

2.3.3.3.1. Cell Phenotyping

As for spleen, MVA-hIL-7-Fc (MVATG18897) effects on restoring immune cell compartments in blood were also investigated in treated CLP mice compared to untreated CLP mice. FIG. 16 shows absolute counts of different cell subsets per μL of blood in CLP mice treated or not with MVA-hIL-7-Fc.

Significantly higher concentration of total CD3⁺ cells was measured in CLP mice administered with MVA-hIL-7-Fc compared with untreated CLP mice (1310 and 794 cells/μL, respectively; FIG. 16A).

Among CD3+ cells, concentration of CD4 T cells increased significantly following MVA-hIL-7-Fc treatment in comparison with untreated CLP mice (577 and 336 cells/μL, respectively; FIG. 16B). Although not significant, CD8 T cell concentration in blood also increased in treated CLP mice compared to untreated ones (595 and 396 cells/μL, respectively; FIG. 16C).

Regarding NKT cells, although detectable at low number in blood, a significantly higher concentration of this cell subset population was measured in CLP mice administered with MVA-hIL-7-Fc than in untreated CLP mice (29 and 18 cells/μL, respectively; FIG. 16D).

Same observation was noticed regarding NK cells in blood. More NK cells were measured in one μL of blood of MVA-hIL-7-Fc-treated CLP mice than in untreated CLP mice (272 and 154 cells/μL, respectively; FIG. 16E).

Some increase of B cell concentration in blood of CLP mice treated with MVA-hIL-7-Fc was observed compared with control CLP mice (1951 and 1318 cells/μL, respectively; FIG. 16F).

Some CD11c⁺ cells increase was also observed in treated CLP mice compared with untreated CLP mice (36 and 14 cells/μL, respectively; FIG. 16G).

Finally, a particularly strong CD11b⁺ cell count increase in CLP mice treated with MVA-hIL-7-Fc was observed when compared to untreated CLP mice (2514 and 1068 cells/μL, respectively; FIG. 16H).

2.3.3.3.2. Cell Activation

Effects of MVA-hIL-7-Fc (MVATG18897) treatment on activation of circulating immune cell subsets were investigated through quantification of CD69-expressing cells by flow cytometry. FIG. 17 shows the number of CD69⁺ cells in different blood cell subsets. MVA-hIL-7-Fc administration resulted in 4-fold increase in CD69⁺ CD4 T cells in CLP mice as compared with untreated CLP mice (18.5 and 4.7 cells/μL, respectively; FIG. 17A).

Activated CD8 T cells in blood were also improved significantly in MVA-hIL-7-Fc-treated CLP mice. Five-fold more CD69⁺ cells per μL of blood were observed in treated CLP mice compared with control CLP mice (69 and 14 cells/μL, respectively; FIG. 17B).

Regarding B cells, a 3.7-fold increase of CD69⁺ B cells were numerated per μL of MVA-hIL-7-Fc-treated CLP mice blood compared with untreated CLP mice (77 and 21 cells/μL, respectively; FIG. 17C).

FIG. 17D illustrates activation status of circulating NK cells. The concentration of CD69⁺ NK cells in blood increased 3.8-fold more in treated CLP mice compared to untreated CLP mice (180 and 48 cells/μL, respectively).

Overall, we show that MVA-hIL-7-Fc enhances significantly T cell compartment, in particular CD4 T cell count, in blood of septic immunosuppressed mice. This result is clinically relevant since T cell number is highly impaired in blood of septic patients. Moreover, activation of several cell subsets, including T, B and NK cells, is enhanced following administration of MVA-hIL-7-Fc in CLP mice. This result is of interest since MVA-hIL-7-Fc treatment may boost the impaired immune system of critically ill septic patients, through activation of circulating immune cells, in order to accelerate control of novel infections and/or prevent them.

2.3.3.4. Effects of MVA-hIL-7-Fc Treatment on T Cell Functions

Functions of immune cells are strongly impaired in septic immunosuppressed patients who become highly sensitive to secondary infections which lead to high mortality rate. It is therefore of importance to restore or boost functions of immune cells in septic patients. Improvement of immune functions, in particular adaptive immunity, by MVA-hIL-7-Fc (MVATG18897) treatment in CLP-induced septic mice was then investigated.

2.3.3.4.1 IFNγ ELISpot Assay

FIG. 18A shows results of an IFNγ ELISpot assay in response to stimulation of total splenocytes with anti-CD3 antibody. MVA-hIL-7-Fc (MVATG18897) treatment resulted in a tremendous increase of the frequency of IFNγ-producing T cells in spleen of CLP mice (1314 spots/10⁵ cells) compared with both Sham and untreated CLP mice groups (17 and 48 spots/10⁵ cells, respectively).

FIG. 18B illustrates the spot sizes (means) in each of the three groups. While the mean size of spots was similar in both Sham and untreated CLP groups (5 and 7×10⁻³ mm², respectively), the mean value obtained with cells of CLP mice treated with MVA-hIL-7-Fc is significantly increased by 2.6-fold (18×10⁻³ mm²), reflecting the capacity of each IFNγ-producing cells from MVA-hIL-7-Fc treated CLP mice to produce larger amounts of IFNγ (spot size being proportional to the amount of IFNγ produced by each cell).

This result is important since T cell IFNγ response in septic patients is described to be impaired. MVA-hIL-7-Fc treatment may indeed boost the adaptive immune system of immunodepressed septic subjects to prevent or fight against infections, thus improving their survival.

2.3.3.4.2 Triple Intracellular Cytokine Staining Assay

Functionality of T cells was also assessed by determining percentage of CD4 and CD8 T cells producing IFNγ, IL2 and/or TNFα following stimulation with anti-CD3 and anti-CD28 antibodies.

FIG. 19A shows the total percentages of CD4 T cells expressing one, two or three of the cytokines among total CD4 T cells. Interestingly, MVA-hIL-7-Fc (MVATG18897) treatment was capable of boosting the CD4 response. A significant higher percentage of cytokines-producing CD4 T cells was measured with splenocytes of MVA-hIL-7-Fc-treated CLP mice compared with untreated CLP mice (27 and 20%, respectively).

Regarding CD8 T cell response (FIG. 19B), impressively the percentage of cytokines-producing CD8 T cells was doubled in CLP mice administered with MVA-hIL-7-Fc when compared with untreated CLP mice (63 and 32%, respectively).

FIG. 20 illustrates the percentage of each of double or triple cytokine-positive CD4 T cell subsets among total CD4 T cell population that are significantly improved by MVA-hIL-7-Fc treatment in the performed ICS assay. When compared to untreated CLP mice, MVA-hIL-7-Fc enhanced significantly IFNγ⁺ TNFα⁺ (0.2 and 0.9%, respectively) and IL2⁺ TNFα⁺ (6.9 and 9.4%, respectively) CD4 T cell subsets (FIG. 20A-B). In parallel, the percentage of triple IFNγ⁺ IL2⁺ TNFα⁺ CD4 T cells was higher in CLP mice administered with MVA-hIL-7-Fc, than in untreated CLP mice (2.3 and 1.0%, respectively; FIG. 20C).

FIG. 21 illustrates the percentage of single, double or triple cytokine positive CD8 T cell subsets among total CD8 T cell population that are significantly improved by the MVA-hIL-7-Fc. Among single-cytokine positive CD8 T cells, percentage of IFNγ+CD8 T cell subset was significantly increased by 2.2-fold in CLP mice treated with MVA-hIL-7-Fc compared with untreated CLP mice (7.0 and 3.2%, respectively) (FIG. 21A). Regarding double-cytokines positive CD8 T cells, a huge increase of IFNγ⁺ TNFα⁺ CD8 T cell subset was induced following treatment with MVA-hIL-7-Fc in CLP mice as compared with untreated CLP mice (18.8 and 2.3%, respectively) (FIG. 21B). Of note, while the three cytokines-positive CD8 T cells were almost undetectable in Sham mice, an important induction of triple IFNγ⁺ IL2⁺ TNFα⁺ CD8 T cells was obtained with splenocytes of CLP mice treated with MVA-hIL-7-Fc compared to cells of untreated CLP mice (7.7 and 1.7%, respectively) (FIG. 21C).

Taken together, data from ELISPOT and ICS assays show the ability of MVA-hIL-7-Fc to improve the functionality of CD4 and CD8 T cells in CLP mice. Thus, MVA-hIL-7-Fc treatment may contribute to help the immune system of critically ill patients to fight or prevent infections by enhancing percentages of T cells producing rapidly cytokines.

2.3.3.4.3. T Cells-Released Cytokines

Functionality of T cells was also assessed by determining level of pro- and anti-inflammatory cytokines produced following in vitro activation of the T cell receptor. Total splenocytes were cultured in anti-CD3 antibody-coated 96-well plates for 24 hours and supernatants were harvested for dosing cytokines.

Measured cytokines were IL-1β, IL-6, TNFα, IFNγ and IL-10 (FIG. 22). Except IL6 which was already significantly higher in supernatants of cells from untreated CLP mice compared to Sham ones (FIG. 22B), supernatants of cells from Sham mice and untreated CLP mice display similar amounts of cytokines (FIG. 22A; C-E). Remarkably, supernatants of cells from MVA-hIL-7-Fc treated CLP mice always displayed a significantly larger amounts of all tested cytokines than untreated CLP and Sham mice (FIG. 22A-E). Of note, level of IL1β is overall lower than the one of other cytokines. Nonetheless, MVA-hIL-7-Fc increased by 6.4-fold the production of this cytokine in supernatants of stimulated splenocytes. For IL6, TNFα, IFNγ, and IL10, detected amounts were respectively increased by 4-, 3-, 5.5- and 2.5-fold in supernatants of cells from MVA-hIL-7-Fc treated CLP mice compared to cells of untreated CLP mice.

This additional read-out clearly suggests the ability of MVA-hIL-7-Fc to improve the capacity of splenocytes from septic mice to produce and secrete pro and anti-inflammatory cytokines when compared to untreated septic ones. One may expect that MVA-hIL-7-Fc may thereby contribute to help the host immune system preventing or fighting infections by improving T cell functionalities.

2.4 In Vivo Comparison of Empty MVA and MVATG18897 in CLP Mice

In order to define the effect of the MVA vector on one side and the effect of the hIL-7-Fc arming, one experiment was performed to compare CLP mice treated by empty MVA (vector activity alone) and MVATG18897 (IL-7 activity+vector activity). Of note, healthy untreated mice were used as positive controls for immunological assays but are not shown there.

Sepsis was induced by CLP in 25 mice at day 0. Half of mice still alive at day 4 were treated by empty MVA and the other half was treated by MVATG18897. Mice were followed up to day 7 and sacrificed to performed immunoassays.

2.4.1 Survival

Survival of mice was followed from DO to D4, i.e. before MVA-hIL-7-Fc (MVATG18897) or MVATGN33.1 (empty MVA) treatment at D4, in all CLP mice (FIG. 23A). Eighteen out of 25 CLP mice (72%) were still alive at D4. At D4, surviving CLP mice were then separated in two groups: CLP mice administered with MVA-hIL-7-Fc at D4 (n=9) and CLP mice treated by empty MVA (n=9). At D7, while 6/9 empty MVA-treated CLP mice (67%) were still alive, all CLP mice treated with MVA-hIL-7-Fc (9/9) survived (FIG. 23B).

This result shows that MVA-hIL-7-Fc improves the survival of CLP-induced septic mice compared to the empty-MVA, suggesting the survival is not only due to MVA activity but due to at least the IL-7 arming or even to the combination of MVA vector and IL-7 arming.

2.4.2. Effect of MVA-hIL-7-Fc Compared to Empty MVA on Blood Cell Subsets

Cell subsets within blood of CLP mice treated by empty MVA and MVA-hIL-7-Fc were assessed by flow cytometry. FIG. 24 shows the cell populations that are significantly increased by MVA-hIL-7-Fc compared to empty MVA. Mean number of circulating CD8+ T cells was almost doubled by the MVA-hIL-7-Fc treatment when compared to the empty MVA treatment in CLP mice (FIG. 24A), as well as the NKT cells (FIG. 24B). Of note, the MVA-hIL-7-Fc treatment strongly improved the number of CD11b+ cells in blood with an increase of more than 3-fold compared to the empty MVA treatment (FIG. 24C).

Overall, these data demonstrate the ability of the IL-7 arming on these immune cell populations in septic mice. The numbers of these cells being altered in a septic context, this suggests the ability of the MVA-hIL-7-Fc to trigger an increased number of such T cells.

2.4.3. Specific Activity of MVA-hIL-7-Fc Compared to Empty MVA on T Cell Functions in CLP Mice.

The functionality of spleen cells stimulated by an anti-CD3 and anti-CD28 was assessed using a triple ICS assay (IFNγ, TNFα and IL2).

FIG. 25 shows the percentage of CD4+ T cells producing 1, 2 or 3 cytokines detected with the ICS, in both groups. FIG. 25A shows the total percentage of CD4+ T cells producing at least one cytokine. MVA-hIL-7-Fc induced a significantly higher percentage of CD4+ T cells producing at least 1 cytokine compared to the empty MVA (44% versus 26% respectively). FIG. 25B shows all CD4+ T cells producing IFNγ, all CD4+ T cells producing TNFα, and all CD4+ T cells producing IL2. Data clearly demonstrates the ability of MVA-hIL-7-Fc, compared to empty MVA, to increase the percentage of each of these cytokine-producing populations (respectively 2.6%, 10.1% and 15.4% for mice treated with empty MVA and 5.6%, 38.7% and 28.8% for mice treated by MVA-hIL-7-Fc). Regarding double and triple-cytokine producing cells, FIG. 25C shows the ones of these populations that are significantly improved by MVA-hIL-7-Fc compared to empty MVA. MVA-hIL-7-FC strongly increased the IFNγ/TNFα (1.22% versus 0.53%), IFNγ/IL2 (0.45% versus 0.34%), IL2/TNFα (20.2% versus 8.7%) and IFNγ/IL2/TNFα (3.6% versus 1.7%) producing CD4+ T cells compared to empty MVA.

FIG. 26 shows the percentage of CD8+ T cells producing 1, 2 or 3 cytokines detected with the ICS, in both groups. FIG. 26A shows the total percentage of CD8+ T cells producing at least one cytokine. MVA-hIL-7-Fc induced a significantly higher percentage of CD8+ T cells producing at least 1 cytokine compared to the empty MVA (64% versus 28% respectively). FIG. 26B shows all CD8+ T cells producing IFNγ, all CD8+ T cells producing TNFα, and all CD8+ T cells producing IL2. Data clearly demonstrates the ability of MVA-hIL-7-Fc, compared to empty MVA, to increase the percentage of each of these cytokine-producing populations (respectively 8.7%, 17.5% and 4.3% for mice treated with empty MVA and 29.6%, 60.5% and 15.1% for mice treated by MVA-hIL-7-Fc). Some significant increases due to MVA-hIL-7-Fc compared to empty MVA were also detected for CD8+ T cells producing only IFNγ (4.6% versus 2.2%) and CD8+ T cells producing only TNFα (27.9% versus 17.5%) and are shown on FIG. 26C. Regarding double and triple-cytokine producing cells, FIG. 26C shows the ones of these populations that are significantly improved by MVA-hIL-7-Fc compared to empty MVA. MVA-hIL-7-Fc strongly increased the IFNγ/TNFα (16.8% versus 4.3%), IL2/TNFα (6.5% versus 1.6%) and IFNγ/IL2/TNFα (7.9% versus 2%) producing CD8+ T cells compared to empty MVA.

Overall, this experiment comparing the effect of MVA-hIL-7-Fc to empty MVA clearly demonstrate a specific activity of the MVA-expressed hIL-7-Fc, as it improves, after one IV injection in CLP mice, the mouse survival, the CD8+ T cell, NKT cell and CD11b+ cell counts in blood and CD4 and CD8+ T cell functionality in spleens.

2.5 Comparison of MVATG18897 and the Recombinant hIL-7-Fc Protein in CLP Mice

In the CLP mouse model, MVATG18897 and hIL-7-Fc protein, were tested in parallel. Both were injected once intravenously 4 days post CLP, and survival was followed until day 7 post-CLP (corresponding to 3 days post injection of the products) and some immune cells from spleen were monitored at day 7 post-CLP.

2.5.1 Survival

After randomization of mice which were still alive at Day 4 (5 in naïve group, 5 in MVA-IL-7-Fc or recombinant IL-7-Fc groups and 4 in CLP untreated mice), it was observed that all mice injected by MVA-hIL-7-Fc (5/5) survived at D7 post-CLP similar to survival observed in the naïve mice (5/5, non-septic mice without any treatment i.e.: positive control of survival) (FIG. 30). In contrast, only 3/4 and 3/5 CLP untreated and recombinant hIL-7-Fc-treated mice respectively survived, indicating a survival advantage associated with treatment by the MVA-hIL-7-Fc and not the recombinant soluble counterpart.

2.5.2 Immune Activities in CLP Mice

2.5.2.1 Activated CD8+ T Cells and B Cells

Activated CD8+ T and B cells displaying CD69 marker of activation were monitored from the spleens of CLP mice that were untreated or treated with MVA-hIL-7-Fc or recombinant hIL-7-Fc (FIG. 31). For CD69+CD8+ T cells (FIG. 31A), CLP mice treated with MVA-hIL-7-Fc displayed significantly higher numbers of these activated cells when compared to untreated CLP mice (about 3-fold increase) and CLP mice treated with the hIL-7-Fc protein (about 2-fold increase). This result demonstrated the stronger ability of MVA-hIL-7-Fc to induce activated CD8+ T cells that should be more reactive in case of new infection, when compared to the recombinant hIL-7-Fc protein. For CD69+ B cells, MVA-hIL-7-Fc treated CLP mice displayed numbers of these cells significantly higher than in untreated mice (about 2-fold increase). hIL-7-Fc protein treated CLP mice tended to display slightly higher numbers of activated B cells than untreated CLP mice but this is not significant, contrary to MVA-hIL-7-Fc. When compared to hIL-7-Fc protein, MVA-hIL-7 induced about 1.5-fold more CD69+ B cell numbers than the protein itself. This suggests a superiority of the MVA-hIL-7-Fc compared to the protein hIL-7-Fc to induce such cells, which will be able to rapidly initiate an immune response in case of secondary infection.

2.5.2.2 Functionality of CD8+ T Cells

The ability of CD8+ T cells from spleens to produce IFNγ and/or TNFα and/or IL-2 after TCR stimulation was assessed for the 3 groups of mice (FIG. 32). Compared to untreated CLP mice, MVA-hIL-7-Fc induced significantly higher percentages of CD8+ T cells producing IFNγ (FIG. 32A) and this is verified for all type of IFNγ producing CD8+ T cells, those producing only IFNγ (FIG. 32B), those producing IFNγ and TNFα (FIG. 32C) and those producing IFNγ and TNFα and IL2 (FIG. 32D). The protein hIL-7-Fc tended to also increase all these cell types, but percentages were significantly higher than in untreated mice only for IFNγ and TNFα producing CD8+ T cells and IFNγ, TNFα and IL2 producing CD8+ T cells. Thus, the effect of hIL-7-Fc was less important than the one of MVA-hIL-7-Fc, suggesting the superiority of MVA-hIL-7-Fc to induce functional CD8+ T cells.

2.6. Optimized MVA-hIL-7-Fc

Two MVA encoding hIL-7-Fc, MVATG18897 and MVATG19791, were designed, constructed, and produced. They both encode hIL-7-Fc under the same promoter (pH5R) but in MVATG19791, codons were optimized for expression of the protein in human cells. MVATG18897 and MVATG19791 were compared in vitro for their expression of the hIL-7-Fc. As shown on FIGS. 33 A and B, CEF or A549 cells were infected by MVATG18897 or MVATG19791. Expressed hIL-7-Fc in cells or in supernatant was detected by Western Blot using anti-IL-7 antibody. In both types of cells, in cells and in supernatants, a higher amount of hIL-7-Fc was detected with MVATG19791, as observed through a larger and more intense band, when compared with MVATG18897.

The amount of produced hIL-7-Fc by the 2 cell types when infected by MVATG18897 or MVATG19791 was determined by ELISA on supernatants of infected cells. As shown in FIG. 33C, the amount of hIL-7-Fc detected with MVATG19791 was superior to the one detected with MVATG18897 (about 1.5-fold more protein detected with MVATG19791 in both cell types), confirming a stronger expression of the protein hIL-7-Fc through the MVATG19791 which was optimized.

In healthy mice, MVATG18897 and MVATG19791 were compared, and they displayed comparable hIL-7-Fc pharmacokinetics and immune activity as expected (data not shown).

2.7 Capacity of the IL-7 Produced by the MVA-hIL-7-Fc to Engage Relevant Immune Pathways and Restore Immune Activity in Immune-Suppressed Patients, Wherein Said Immune Suppression is not Induced by Sepsis

In order to illustrate the beneficial value of the MVA-hIL-7-Fc at restoring immune activities in different immune-suppressed clinical settings, a series of analysis were performed using whole blood and/or PBMC obtained from different cohorts of patients.

The source of IL-7-Fc to be tested was typically obtained after infection of primary human hepatocytes by the MVATG18897 at a MOI of 5, and supernatants were harvested 24 h after infection. Quantification of hIL-7-Fc in supernatants was performed using the ELISA hIL-7 kit and stored at −70° Celsius. Negative control-supernatants included those collected after infection with MVA empty (MVATGN33.1) and/or uninfected cells collected under same conditions as for MVA-hIL-7-Fc.

2.7.1. ICU Covid-19 Immuno-Suppressed Patients

2.7.1.1 pSTAT5 Expression in CD3+ T Cells

The cytokine IL-7 recognizes the IL-7 receptor mediating the signaling pathway. IL-7 signaling is initiated through Janus Kinase 1,3 and phosphoinositide 3 (PI3k) resulting in phosphorylation of signal transducer and activator of transcription 5 (STAT5). Engagement of this early pathway by IL-7 ultimately leads to increase of T cell survival and proliferation. Signal transduction through STAT5 phosphorylation was assessed after stimulation with supernatant from MVA-hIL-7-Fc infection to demonstrate activity of the produced IL-7-Fc. FIG. 34 shows pSTAT5 expression detected in CD3+ T cells after 10 min ex vivo stimulation of whole blood of COVID19+ patients with supernatants from MVATG18897 or MVATGN33.1 infected cells or uninfected ones expressed as ratio (values obtained with supernatants of MVATG18897 or MVATGN33.1 infection or uninfected cells were normalized by values of un-stimulated condition). Three different supernatant dilutions (1/2.5, 1/5 and 1/10) from the MVAs infections and one (1/2.5) of uninfected cells were tested. As shown in the figure, MVA-hIL-7-Fc supernatant induced a strong increase of pSTAT5 expression with a dilution effect (multiplied by 6.5, 5.7 and 4.9 for dilutions 1/2.5, 1/5 dilution and 1/10 respectively) whereas no increase of pSTAT5 expression was observed after stimulation with empty MVA or uninfected supernatants.

These data demonstrate that hIL-7-Fc produced by the MVA-hIL-7-Fc was recognized by the IL-7 receptor and can initiate IL-7 signaling through the phosphorylation of STAT5. The observed hIL-7-Fc-mediated signaling is expected to increase proliferation of T lymphocytes of COVID19+ patients and improve their survival.

2.7.1.2 Total IFNγ+ and IFNγ+ TNFα+IL-2+ Produced by CD4+ T Cells

Functionality of CD4+ T cells was assessed by intracellular staining of IFNγ or IFNγ TNFα IL-2 produced by CD4+ T cells after stimulation with Duractive 1 and supernatant from MVA-hIL-7-Fc infection.

FIGS. 35A and 35B show the percentage of CD4+ T cells producing total IFNγ+ or IFNγ+ TNFα+IL-2+ respectively after 3 hours ex vivo stimulation of whole blood of COVID19+ patients with supernatants of MVATG18897 or MVATGN33.1 or uninfected cells expressed as ratio (values of Duractive 1+supernatant of MVATG18897 or MVATGN33.1 infection or uninfected cells were normalized by the value of Duractive 1 condition). Supernatants containing hIL-7-Fc increased the percentage of CD4+ T cells producing total IFNγ (FIG. 35A) with a dilution effect (Ratio 1.4 for 1/2 dilution and 1.1 for 1/10 dilution). A contribution of supernatant from empty MVA infection in the first dilution (Ratio 1.18 for 1/2 dilution and 1.0 for 1/10 dilution) was observed suggesting an effect of the viral vector itself. A minor effect of uninfected cell supernatant was observed (Ratio 1.09) compared to other supernatants. hIL-7-Fc in supernatant increased production of IFNγ+ TNFα+IL-2+ by CD4+ T cells (FIG. 35B) with a dilution effect (Ratio 1.65 for 1/2 dilution and 1.18 for 1/10 dilution). A similar effect was observed after stimulation of whole blood cells with supernatant from empty MVA and uninfected cells at dilution one-half (Ratio 1.3 for both) suggesting an effect of a soluble molecules produced by the cultured cells. No effect of empty MVA supernatant was observed at dilution 1/10.

The capacity of T cells to produce IFNγ is described as reduced in COVID19+ patient in ICU. The collected data indicated that the IL-7-Fc produced by MVA-hIL-7-Fc promoted the production of IFN-γ and is thus expected to boost the adaptative immune response of immunosuppressed COVID19+ patients to fight infections and to improve their survival.

2.7.2. Senescent Trauma Patients

2.7.2.1 CD57 Expression in CD8 T Cells

We analyzed the presence of CD57, a well-known T cell senescent marker in the studied HIPAGE cohort. As expected and shown in FIG. 36, CD8 T cells from senescent controls as well as hip fractured patients highly expressed CD57. Furthermore, a slight increase of this marker was observed in the hip fractured patients.

2.7.2.2 pSTAT5 Expression in CD3+ T Cells

Signal transduction through STAT5 phosphorylation was assessed after stimulation with supernatant from MVA-hIL-7-Fc infected cells. FIG. 37A showed the pSTAT5 expression in CD3+ cells after 10 min ex vivo stimulation of thawed PBMCs with supernatants of MVATG19791 (SN IL-7-Fc) or MVATGN33.1 (SN N33) or after no stimulation.

While pSTAT5 was not expressed by unstimulated CD3+ cells, there was a slight increase in CD3+ cells stimulated with supernatant of empty MVA only in hip fractured patients. By contrast, MVA-hIL-7-Fc supernatant induces a far greater increase of pSTAT5 expression in CD3+ cells for both patient groups.

These data demonstrated that hIL-7-Fc produced in supernatant was recognized by IL-7 receptor and can initiate IL-7 signaling through the phosphorylation of STAT5. Thus, hIL-7-Fc mediated signaling is thus expected to increase T cell proliferation.

2.7.2.3 T Cell Proliferation

Proportion of proliferating CD3+ cells was assessed after stimulation with supernatant from MVA-hIL-7-Fc infected cells. FIG. 37B shows the proliferation induction in CD3+ cells after 5 days ex vivo stimulation of thawed PBMCs with supernatants of MVATG19791 or of MVATGN33.1 or after no stimulation.

While T cell proliferation was not induced in unstimulated CD3+ cells and in CD3+ cells stimulated with supernatant of empty MVA, MVA-hIL-7-Fc supernatant strongly induced T cell proliferation, specifically in hip fracture patient group.

hIL-7-Fc produced in supernatant increased T cell proliferation and is thus expected to participate to the immune restauration in senescent hip fractured patients after surgery.

2.7.3. ICU Trauma Patients

Functionality of CD4+ T cells from heavy polytraumatic patients was assessed by intracellular staining of IFNγ or IFNγ TNFα IL-2 produced by CD4+ T cells after stimulation with PMA/ionomycin or anti-CD3/CD28 antibodies, and supernatant from MVA-hIL-7-Fc infection.

FIGS. 38A and 38B show the percentage of CD4+ T cells producing total IFNγ+ or IFNγ+ TNFα+IL-2+ respectively after 5 hours ex vivo stimulation of PBMCs from trauma patients with a strong (PMA/lonomycin) or a moderate (anti-CD3/CD28) activation. Stimulations were performed in presence of supernatants from MVATG19791 or MVATGN33.1 infected primary hepatocytes.

Supernatant containing hIL-7-Fc highly increased the percentage of CD4+ T cells producing total IFNγ (FIG. 38A). After a strong T cell activation with PMA/lonomycin, 18% of CD4+ T cells expressed IFNγ in presence of MVA-hIL7 supernatant, compared to 10.5% in control group. A contribution of supernatant from empty MVA infection was measured (13.7%). Same observations were done after a moderate activation with anti-CD3/CD28 between SN IL7 and control groups (3.8% and 2.2% respectively). No IFNγ expression was observed in unstimulated cells.

More specifically, hIL-7-Fc in supernatant increased the percentage of polyfunctional CD4+ T cells expressing IFNγ+, TNFα+ and IL-2+ (FIG. 38B). After a strong T cell activation with PMA/lonomycin, 13.5% of CD4+ T cells expressed IFNγ in presence of MVA-hIL7 supernatant, compared to 8% in control group. A contribution of supernatant from empty MVA infection was measured (10.5%). Same observations were done after a moderate activation with anti-CD3/CD28 between SN IL7 and control groups (2% and 0.9% respectively). No IFNγ expression was observed in unstimulated cells.

These data demonstrated that hIL-7-Fc produced in supernatant increased the capacity of T cells to produce IFNγ and thus expected to participate to the restauration of the adaptative immune response in immunosuppressed patients after heavy polytraumatic shock.

2.7.3. ICU Heavy Surgery Patients

Functionality of CD4+ T cells from heavy surgery patients was assessed by intracellular staining of IFNγ or IFNγ TNFα IL-2 produced by CD4+ T cells after stimulation with PMA/ionomycin or anti-CD3/CD28 antibodies, and supernatant from MVA-hIL-7-Fc infection.

FIGS. 39A and 39B show the percentage of CD4+ T cells producing total IFNγ+ or IFNγ+ TNFα+IL-2+ respectively after 5 hours ex vivo stimulation of PBMCs from heavy surgery patients with a strong (PMA/lonomycin) or a moderate (anti-CD3/CD28) activation. Stimulations were performed in presence of supernatants from MVATG19791 or MVATGN33.1 infected primary hepatocytes.

Supernatant containing hIL-7-Fc highly increased the percentage of CD4+ T cells producing total IFNγ (FIG. 39A). After a strong T cell activation with PMA/lonomycin, 12.7% of CD4+ T cells expressed IFNγ in presence of MVA-hIL7 supernatant, compared to 5.5% in control group. Same observations were observed after a moderate activation with anti-CD3/CD28 between SN IL7 and control groups (3.8% and 2.2% respectively). No IFNγ expression was observed in unstimulated cells.

More specifically, hIL-7-Fc in supernatant increased the percentage of polyfunctional CD4+ T cells expressing IFNγ+, TNFα+ and IL-2+ (FIG. 39B). After a strong T cell activation with PMA/lonomycin, 8.2% of CD4+ T cells expressed IFNγ in presence of MVA-hIL7 supernatant, compared to 2.5% in control group. Same observations were observed after a moderate activation with anti-CD3/CD28 between SN IL7 and control groups (1.5% and 0.6% respectively). No IFNγ expression was observed in unstimulated cells.

These data demonstrated hIL-7-Fc produced in supernatant increased the capacity of T cells to produce IFNγ and thus expected to participate to the restauration of the adaptative immune response in immunosuppressed patients after heavy surgery procedures, to fight secondary infections and improve their survival.

2.8 General Conclusions on Results

Main effects of the tested products can be summarized as follow:

MVA-hIL-7-Fc induces the production of circulating hIL-7 which is clearly detected in blood rapidly after injection and still detected up to 4 days after 1 intravenous injection. This injection also induced the transient production of circulating IFNγ which is detected shortly after injection. This IFNγ production is induced by the MVA vector itself.

In naïve mice, an increase in splenocyte number is observed after one IV injection of MVA-hIL-7, in particular the numbers of CD4+ T cells, CD8+ T cells, neutrophils, myeloid dendritic cells (mDC), and monocytes (all three sub-populations LyC^{high}, Ly6C^{int} and Ly6C^{low}). Among CD4+ and CD8+ T cells from the spleen, MVA-hIL-7-Fc increases in particular the number of effector memory and central memory CD4+ T cells as well as effector memory, central memory and acute effector CD8+ T cells. Within these CD4+ and CD8+ T cells, the expression of the anti-apoptotic protein Bcl2 is also improved following one IV injection of MVA-hIL-7-Fc. An increase in Bcl2 expression in thymic cells and a modification of thymic proportions (decrease in double positive CD4+/CD8+ and increase in single positive CD4+ or CD8+) were also observed after MVA-hIL-7-Fc injection, this effect being mainly mediated by the MVA vector itself (similar observations with empty MVA). All these activities described in naïve mice clearly demonstrate the ability of MVA-hIL-7-Fc, linked to the contribution of either of both expressed IL-7 and the empty MVA backbone, to activate the immune system by improving numbers of immune cells involved in the fight against infections, including mature ones, and improving gene survival expression in these key cells.

In naive mice, MVA-hIL-7-Fc was shown to induce immune activities in lungs, this activity being in part due to the backbone and in part due to the arming hIL-7-Fc. It improves the numbers of activated T and NK cells.

In addition, in a septic mouse model, MVA-hIL-7 was shown to improve survival compared to untreated mice as well as mice treated with the empty MVA, suggesting the absolute necessity to arm the MVA with hIL-7-Fc to reach protection. In this same model, MVA-hIL-7-Fc was proven to restore normal spleen cell counts and restore at least partially T cell counts, in particular CD8+ T cell ones. In blood, it improved cell counts of several immune cells. In addition, it was shown to also activate several immune cell populations in spleen and blood of septic mice, these cells being then ready to fight new infectious attacks. MVA-hIL-7-Fc was also shown to be able to boost the functionality of T cells, in particular by improving the frequency of IFNγ-producing cells, each cell being also able to produce higher amounts of IFNγ. It also boosted the frequency of cells able to produce TNFα, IL2, or 2 or 3 cytokines among IFNγ, TNFα and IL2. The effect on immune cells activation appeared to be mainly mediated by the MVA vector itself, whereas all other activities appeared mainly specific of the MVA encoding the hIL-7-Fc.

When compared to the activity of a recombinant hIL-7-Fc, similar to the one encoded within the MVA-hIL-7-Fc, it was shown to be superior. As examples, in naïve healthy mice, it induced significantly higher numbers of activated T and NK cells in lungs and it induced also significantly higher percentages of CD8+ T cells producing cytokines after TCR stimulation, both in spleens and lungs. In CLP mice, MVA-hIL-7-Fc was responsible for a higher survival rate than the hIL-7-Fc protein and induced stronger immune activities in spleens. As an example, MVA-hIL-7-Fc induced higher numbers of activated CD8+ T cells and B cells and was superior to hIL-7-Fc protein for the induction of CD8+ T cells producing IFNγ (+/−other cytokines).

In addition, we have demonstrated using ex vivo assays based on whole blood and/or PBMC from three cohorts of immune-suppressed clinical situations (ICU Covid-19 immuno-suppressed patients, senescent trauma patients and ICU trauma patients) that the IL-7-Fc produced by the MVA-hIL-7-Fc can restore a number of immunological impairments.

Overall, the vectorized form of IL-7 such as obtained using the MVA-hIL-7-Fc induces a wide array of immune modulations capable to restore and/or to improve multiple immune-suppressed pathways and functions in a variety of ICU-clinical situations (immune depression induced by sepsis, trauma, burn, major surgery and/or coronavirus) or in elderly population (immune depression induced by senescence). The restoration and/or improvement of the immune response helps patients to fight and/or to prevent infections and to accelerate global recovery. These results clearly demonstrate the interest of administrating a non-replicative viral vector expressing IL-7 in a context of immunosuppression.

### BIBLIOGRAPHIC REFERENCES

Adra et al., 1987, Gene 60: 65-74
Antoine et al., 1998, Virol. 244: 365-96
Capasso et al., 2014, Viruses, 6, 832-855
Cervasi et al., 2015, J. of Virology, 87(17):9420-9430
Chakrabarti et al. (1997, Biotechniques 23: 1094-7
Chen et al., 2000, J Immunol, 165:6496-6503
Chen et al., 2015, J. of Virology, 84(20): 10522-10532
Choi et al., 2016, Clin Cancer Res; 22(23): 5898-5908
Chroboczek et al., 1992, Virol. 186:280
Chu et al., 1997, J. Exp. Med., 186: 1623
Dicks et al., 2012, PLoS One.; 7(7): e40385
Dudareva et al., 2009, Vaccine 27: 3501
Erbs et al., 2008, Cancer Gene Ther. 15(1): 18-28
Fallaux et al., 1998, Human Gene Ther. 9: 1909-1917
Frangois et al., 2018, JCI Insight.; 3(5):e98960
Gao et al., 2015, Int J Mol Sci.; 16(5): 10267-10280
Graham et al., 1997, J. Gen. Virol. 36: 59-72
Hamers et al., 2015, Minerva Anestesiol., 81(4):426-439
Hammond et al, 1997, J. Virol Methods 66: 135-8
Heftrig et al., 2017, Mediat Inflamm, article ID 2608349
Jia et al., 2019, Eur J Trauma Emerg S, https://doi.org/10.1007/s00068-019-01271-6
Kared et al., 2016, J Immunol Immunother, 65(4):441-52
Kovac et al., 2005, Exp Gerontol., 40(7):549-55
Kritsch et al., 2005, J. Chromatogr. Anal. Technol. Biomed. Life Sci., 822: 263-70
Kumar and Boyle, 1990, Virology 179: 151-8
Lusky et al., 1998, J. Virol 72: 2022
Maniatis et al., 1989, Laboratory Manual, Cold Spring Harbor Laboratory Press, Cold Spring Harbor N.Y. or subsequent editions
Mayr et al., 1975, Infection 3: 6-14
Meisel et al., 2009, Am J Respir Crit Care Med., 180(7):640-8
Monneret et al., 2020, Intensive Care Med, 46:1764-1765
N'Guyen et al., 2016, Molecular Therapy Oncolytics, 3, 15021
Nam et al., 2010, Eur. J. Immunol., 40: 351-358
Olivier et al., 2010, mAbs 2(4): 405-15
PCR protocols—A guide to methods and applications, 1990, Innis, Gelfand, Sninsky and White, Academic Press
Peruzzi et al., 2009, Vaccine 27: 1293
Peters van Ton et al., 2018, Front Immunol, 9: 1926
Ponchel et al., 2011 Clinica Chimica Acta 412, 7-16
Restagno et al., 2016, PLoS One, 11(8):e0162109
Seo et al., 2014 J. of Virology 88(16): 8998-9009
Shindo et al., 2015; 43(4): 334-343
Sutter and Moss, 1992, Proc. Natl. Acad. Sci. USA 89: 10847-51
Takeyama et al., 2004, Critical Care 20048 (Suppl 1):P207
Tartaglia et al., 1992, Virol. 188(1):217-32
Tesfay et al., 2013, J. of Virology, 87(7): 3752-3759
The Science and Practice of Pharmacy, A. Gennaro, Lippincott, Williams & Wilkins
Thomson et al., 2019, Military Medical Research, 6:11
Tritel et al., 2003, J. Immunol., 171: 2358
Unsinger et al., 2010, J Immunol; 184:3768-3779
Vanloan et al., 2017, J. Immunol. Res., Article ID 4807853
Venet and Monneret, 2017, Nat Rev Nephrol., 14(2):121-137
Venet et al., 2012, J Immunol November 15, 189 (10) 5073-5081
Yamamoto et al., 2017, Cancer Sci 108 (2017) 831-837
U.S. Pat. No. 5,168,062
U.S. Pat. No. 5,879,924
WO94/28152
WO97/00326
WO98/02522
WO98/10088
WO01/66137
WO03/000283
WO03/046124
WO03/053463
WO03/104467
WO2005/007840
WO2005/042728
WO2005/071093
WO2007/056847
WO2007/077256
WO2007/147528
WO2007/147529
WO2008/114021
WO2008/129058
WO2008/138533
WO2009/004016
WO2009/073103
WO2009/073104
WO2009/100521
WO2009/105084
WO2009/136977
WO2010/086189
WO2010/130753
WO2010/130756
WO2012/001075
WO2013/022764
WO2017/037523

1.-25. (canceled)

26. A method for treating an immune depression induced by sepsis, burn, trauma, major surgery, senescence, coronavirus, or any combination thereof in subject in need thereof, comprising one or more administration(s) to the subject of:
(i) a non-propagative viral vector comprising a nucleic acid molecule encoding at least a polypeptide having an IL-7 activity, or
(ii) a composition comprising a non-propagative viral vector comprising a nucleic acid molecule encoding at least a polypeptide having an IL-7 activity, and an acceptable pharmaceutical vehicle.

27. The method according to claim 26, wherein said viral vector is selected from the group consisting of poxviruses, adenoviruses, adenovirus associated viruses, vesicular stomatitis viruses, measle virus, poliovirus, Maraba Virus, and viral like particles.

28. The method according to claim 26, wherein said viral vector is a non-propagative poxvirus.

29. The method according to claim 28, wherein said non-propagative poxvirus is a non-propagative vaccinia virus.

30. The method according to claim 29, wherein said non-propagative poxvirus is a modified vaccinia virus Ankara (“an MVA”).

31. The method according to claim 26, wherein said viral vector is an adenovirus.

32. The method according to claim 31, wherein said adenovirus is a human adenovirus selected from the group consisting of species B, C, and D.

33. The method according to claim 32, wherein said human adenovirus is selected from the group consisting of adenoviruses of serotypes 5, 11, 26, and 35.

34. The method according to claim 26, wherein said polypeptide is selected from the group consisting of the murine IL-7, the human IL-7, the murine IL-7 fused with a Fc domain, and the human IL-7 fused with a Fc domain.

35. The method according to claim 34, wherein:
said murine IL-7 comprises an amino acid sequence having at least 70% identity with the amino acid sequence shown in SEQ ID NO:1;
said human IL-7 comprises an amino acid sequence having at least 70% identity with the amino acid sequence shown in SEQ ID NO:2;
said murine IL-7 fused with a Fc domain comprises an amino acid sequence having at least 70% identity with the amino acid sequence shown in SEQ ID NO:3; or
said human IL-7 fused with a Fc domain comprises an amino acid sequence having at least 70% identity with the amino acid sequence shown in SEQ ID NO:4.

36. The method according to claim 34, wherein:
said murine IL-7 comprises an amino acid sequence having at least 80% identity with the amino acid sequence shown in SEQ ID NO:1;
said human IL-7 comprises an amino acid sequence having at least 80% identity with the amino acid sequence shown in SEQ ID NO:2;
said murine IL-7 fused with a Fc domain comprises an amino acid sequence having at least 80% identity with the amino acid sequence shown in SEQ ID NO:3; or
said human IL-7 fused with a Fc domain comprises an amino acid sequence having at least 80% identity with the amino acid sequence shown in SEQ ID NO:4.

37. The method according to claim 26, wherein said viral vector comprises the regulatory elements necessary for the expression of a polypeptide having an IL-7 activity in a host cell or subject.

38. The method according to claim 28, wherein said composition comprises a dose of non-propagative poxvirus comprised between approximately 10⁶ and approximately 10¹² pfu.

39. The method according to claim 31, wherein said composition comprises a dose of non-propagative adenovirus comprised between approximately 10⁶ and approximately 10¹⁴ vp.

40. The method according to claim 26, wherein said composition is administered via parenteral route.

41. The method according to claim 26, wherein said composition is administered (i) at an early stage within the first month after the beginning of the immune depression phase, (ii) within a period of time varying from approximately 1 month to approximately 6 months after the beginning of the immune depression phase, (iii) at a later stage within 6 months to 6 years after the beginning of the immune depression phase, or (iv) any combination thereof.

42. The method according to claim 26, wherein said use comprises administration of said non-propagative viral vector or composition between 1 and times after the beginning of the immune depression phase.

43. The method according to claim 26, wherein said immune depression is induced by sepsis.

44. The method according to claim 26, wherein said immune depression is induced by coronavirus.

45. The method according to claim 26, wherein said administration increases the functional innate and/or adaptive immune system in a subject administered with said non-propagative viral vector or composition compared to a subject not administered with said non-propagative viral vector or composition.

46. The method according to claim 26, wherein said administration increases the level of at least one type of cells associated with immunity selected from the group consisting of CD4 T cells, CD8 T cells, B cells, NKT cells, NK cells, dendritic cells, monocytes, and neutrophils, in a subject administered with said non-propagative viral vector or composition compared to a subject not administered with said non-propagative viral vector or composition.

47. The method according to claim 26, wherein said administration increases the level and/or the percentage of at least one type of activated cells associated with immunity selected from the group consisting of activated CD4 T cells, activated CD8 T cells, activated B cells, and activated NK cells, in a subject administered with said non-propagative viral vector or composition compared to a subject not administered with said non-propagative viral vector or composition.

48. The method according to claim 26, wherein said administration increases the level of cytokines of at least one type selected from the group consisting of IFNγ, TNFα, IL-6, and IL-1β, in a subject administered with said non-propagative viral vector or composition compared to a subject not administered with said non-propagative viral vector or composition.

49. The method according to claim 26 for the treatment of an immune depressed subject displaying one or more biomarkers associated with the decrease of the number of cells associated with immunity and/or the level of activated cells associated with immunity.

50. The method according to claim 49, wherein said one or more biomarkers is/are selected from the group consisting of HLA-DR expression on circulating monocytes, circulating IL-10, absolute CD3 T cell count, several immunosuppressive lymphocyte subpopulations measurements including regulatory T cells, and expression of inhibitory receptors.

51. The method according to claim 50, wherein said inhibitory receptors are selected from the group consisting of PD-1, PD-L1, CTLA-4, Lag3, and BTLA.


SEQUENCE LISTING (4 sequences)

SEQ ID NO: 1 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 2 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 381 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 4 - protein, 405 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK